(19) United States
(12) Patent Application Publication
(10) Pub. No.: US 2026/0200927 A1
(43) Pub. Date: Jul. 16, 2026

(54) NON PEPTIDERGIC AGONISTS OF OXYTOCIN RECEPTOR

(71) Applicants: UNIVERSITE DE STRASBOURG, STRASBOURG (FR); CENTRE NATIONAL DE LA RECHERCHE SCIENTIFIQUE, PARIS (FR)

(72) Inventor: Marcel HIBERT, ESCHAU (FR)

(21) Appl. No.: 19/134,001

(22) PCT Filed: Nov. 30, 2023
(86) PCT No.: PCT/EP2023/083749

§ 371 Date: May 29, 2025

(30) Foreign Application Priority Data
Nov. 30, 2022 (EP) 22306768.7

(51) Int. Cl. C07D 487/04; A61K 31/17; A61K 31/551; A61K 45/06

(52) U.S. Cl. CPC C07D 487/04; A61K 31/17; A61K 31/551; A61K 45/06

(57) ABSTRACT

The present disclosure concerns a non peptidergic agonist of oxytocin receptor, a pharmaceutical composition comprising such a compound and a pharmaceutically acceptable vehicle, and notably said compound for the treatment of all known pathologies where oxytocin has a beneficial effect such as autism spectrum disorders, in particular for the treatment of social interaction impairments in autism spectrum disorders.

The present invention concerns a non peptidergic agonist of oxytocin receptor, a pharmaceutical composition comprising such a compound and a pharmaceutically acceptable vehicle, and notably said compound for the treatment of all known pathologies where oxytocin has a beneficial effect such as autism spectrum disorders, in particular for the treatment of social interaction impairments in autism spectrum disorders, and alcohol addiction.

Millions of people worldwide suffer from some form of mental, neurological or behavioral disorders. Among them, many psychiatric disorders are characterized by a fundamental disruption of social behavior. Common examples include autism spectrum disorder (ASD) and social anxiety disorder (SAD). Additionally, several disorders have social withdrawal as a secondary symptom; examples include depression, schizophrenia, major depressive disorder (MDD) and substance use disorders.

As described in the World Health Organization's International Classification of Diseases ICD-11, ASD is characterized by persistent deficits in the ability to initiate and to sustain reciprocal social interaction and social communication, and by a range of restricted, repetitive, and inflexible patterns of behavior, interests or activities that are clearly atypical or excessive for the individual's age and sociocultural context. The onset of the disorder occurs during the developmental period, typically in early childhood, but symptoms may not become fully manifest until later, when social demands exceed limited capacities. Deficits are sufficiently severe to cause impairment in personal, family, social, educational, occupational or other important areas of functioning and are usually a pervasive feature of the individual's functioning observable in all settings, although they may vary according to social, educational, or other context. Individuals along the spectrum exhibit a full range of intellectual functioning and language abilities.

Approximately 1/100 children are diagnosed with autism spectrum disorder around the world, and prevalence estimates are increasing over time.

At present there are no approved drugs that target core symptoms of ASD, in particular disturbance of social interactions and communication, repetitive behaviors and restricted social interest. Currently prescribed drug-treatments in relation with these disorders (typically selective serotonin reuptake inhibitors (SSRIs), serotonin and noradrenaline reuptake inhibitors (SNRIs), stimulants and antipsychotics) are only targeting some associated symptoms (anxiety, depression, etc.) and have at best limited efficacy, poor compliance and vast side effect profiles, because they are indeed inefficient to address said core symptoms of ASD.

Researches have suggested that a promising target for treating these diseases is the neuropeptide oxytocin (OT). Animal studies showed that OT positively modulates a wide variety of social interactions, including maternal behavior, courtship, sexual behavior and peer-to-peer interaction. Results of human studies, in which OT is usually administered intranasally, showed that OT can increase trust and co-operation, improve social memory and decrease social fear. Moreover, recent clinical trials indicated that administration of OT to a person with autism and social anxiety can restore some aspects of social functioning. The OT receptor therefore has potential for drug discovery aimed at alleviating serious psychiatric disorders.

OT has also a beneficial effect on pathologies, diseases, conditions, disorders, such as pain, drug and/or alcohol addiction, depression, post-traumatic stress, anxiety, eating disorders, notably anorexia, bulimia and Prader-Willi syndrome, schizophrenia, neurodevelopmental disorders, social interaction disorders, borderline behavior, ageing of the skin, muscles and bones.

However, OT is not suitable for a drug development, notably for CNS disorder, since OT is a big cyclic peptide (MW over 1000 Da) that does not survive primary metabolism, has a short physiological half-life (about 3-minute half-life in blood) and does not readily penetrate the blood brain barrier.

Furthermore, OT is not specific since it binds, further to OT-receptors, to vasopressin 1 a receptor (V1aR). In fact, the OT receptors have a high degree of homology with the V1a receptors, meaning that selectivity can be extremely challenging.

Therefore, the development of new compounds that overcome the above-mentioned drawbacks is strongly needed.

Accordingly, it is an object of the present invention to provide new compounds that target the core symptoms of ASD.

Another object of the present invention is to provide pharmaceutical compositions and compounds for their use to treat autism, autism spectrum disorders, in particular for the treatment of social interaction impairments in autism spectrum disorders, pain, drug and/or alcohol addiction, depression, post-traumatic stress, anxiety, eating disorders, notably anorexia, bulimia and Prader-Willi syndrome, schizophrenia, neurodevelopmental disorders, social interaction disorders, borderline behavior, ageing of the skin, muscles, bones, erectile dysfunction, and any disease for which oxytocin is likely to have a beneficial effect.

Another object of the present invention is to provide dermatologic and/or cosmetic compositions, and methods to reduce ageing of the skin.

Another object of the present invention is to provide methods to facilitate erection, lactation, childbirth, breastfeeding, and/or post-partum period, comprising a step of administering to a subject in need thereof a compound as defined above.

Another object of the present invention is to provide compounds that have improved activity on endogenous OT and/or improved selectivity for endogenous OT, in particular over vasopressin V1a, V1b and/or V2 receptors.

Another object of the present invention is to provide compounds that are non-peptide small molecules.

Thus, in one aspect, the present invention relates to a compound of following formula (I):

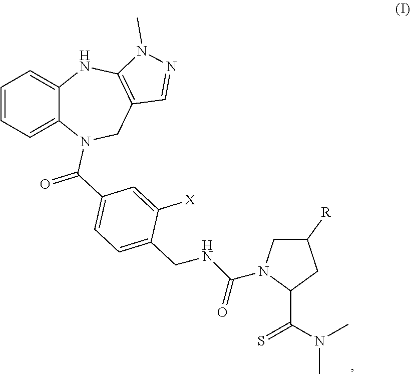

wherein:
X is chosen from —H, halogens, —(C₁-C₆)-alkyl groups, —O—(C₁-C₆)-alkyl groups and —O—CF₃, in particular -Me or —Cl, more particularly -Me;
R is chosen from the group comprising:
   —(C₁-C₆)-alkyl groups, in particular -Me;
   —O—(C₁-C₄)-alkyl groups, in particular —OMe;
   —S—(C₁-C₄)-alkyl groups, in particular —SMe;
   —O—(C₂-C₄)-alkyl-OH groups, in particular —O—(CH₂)ᵢ—OH, with i ranging from 2 to 4, i being more particularly 2;
   —O(—(C₂-C₄)-alkyl)ₙ-OH groups, in particular —O(—CH₂—CH₂)ₙ—OH, with n ranging from 2 to 4, n being more particularly 2;
   —O—(C₂-C₄)-alkyl-O—(C₁-C₄)-alkyl groups, in particular —O—(CH₂)ᵢ—OMe, with i ranging from 2 to 4, i being more particularly 2;
   —O(—(C₂-C₄)-alkyl)ₘ-(C₁-C₄)-alkyl groups, in particular —O(—CH₂—CH₂)ₘ—OMe or —O(—CH₂—CH₂)ₘ—OEt, with m ranging from 2 to 4, m being more particularly 2;
   —O—(C₁-C₄)-alkyl-Ar, in particular —O—(CH₂)ⱼ-Ar, with j ranging from 1 to 4, j being more particularly 1, and Ar being an aryl group being optionally substituted by at least one R″ group, in particular at least one —OH group, said —O—(C₁-C₄)-alkyl-Ar group being for example of the following formula:

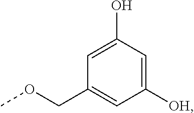

R″ being chosen from the group comprising:
   —OH;
   a —O—(C₁-C₆)-alkyl or —O-cyclo-(C₃-C₆)-alkyl, in particular a —O-cyclopropyl;
   a —(C₁-C₆)-alkyl or -cyclo-(C₃-C₆)-alkyl, in particular a cyclopropyl;
   —(C₁-C₆)-alkyl-W groups, in particular —(CH₂)ₖ—W groups, said groups being optionally further substituted by at least a —W′ group, with k ranging from 1 to 6, k being more particularly 1 or 2, and W and W′ being ORₐ, with Rₐ representing —H or —(C₁-C₄)-alkyl, in particular H or Me, or a PEG group, in particular of following formula —(CH₂—CH₂—O)ₗ—H, with l ranging from 1 to 10, in particular from 2 to 4, for example 2; or W and W′ being NR_{b}H, with R_{b} representing —H, a —(C₁-C₄)-alkyl group, or a —(C₁-C₄)-alkyl-Ar′, with Ar′ being an aryl or heteroaryl group, in particular a furanyl;
   —(C₁-C₆)-alkyl-Y—Z groups, in particular —(CH₂)ₗ—Y—Z, with l ranging from 1 to 6, 1 being more particularly 1 or 2, Y being —O—, —NH— or being absent, and Z being chosen from saturated heterocyclic groups, in particular morpholinyl, tetrahydropyranyl, imidazolidinyl, said saturated heterocyclic groups being optionally substituted by at least one ═O group, representing for example imidazolidine-2,4-dione; and heteroaryl groups, in particular tetrazolyl;
   An aryl or heteroaryl group, in particular a pyrazolyl, said group being optionally substituted by at least one R_{c} group chosen from —(C₁-C₆)-alkyl and —NH₂ groups;
   a —(C₀-C₆)-alkyl-C(═O)NH₂ or —(C₀-C₆)-alkyl-S(═O)₂NH₂ group, in particular a —C(═O)NH₂ or —(CH₂)₂—S(═O)₂NH₂ group;
Halogens, in particular —F;
—CF₃;
or R is —F and its geminal hydrogen is replaced by —F, R forming with the carbon atom C to which it is bound and its —F replaced geminal hydrogen a CF₂ residue;
or an enantiomer, diastereoisomer, and/or a pharmaceutically acceptable salt or solvate thereof.

In another aspect, the present invention relates to a compound of following formula (I):

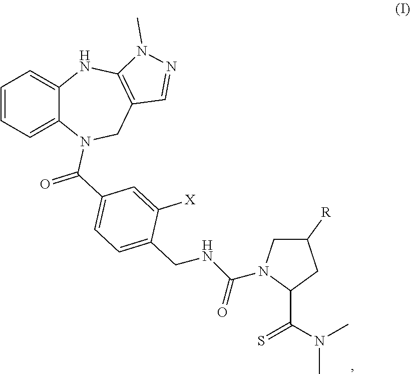

wherein:
X is chosen from —H, halogens, —(C₁-C₆)-alkyl groups, —O—(C₁-C₆)-alkyl groups, —S—(C₁-C₆)-alkyl groups and —O—CF₃, —S—CF₃, in particular -Me or —Cl, more particularly -Me;
R is chosen from the group comprising:
   —(C₁-C₆)-alkyl groups, in particular -Me;
   —(C₁-C₆)-alkyl-V groups, in particular —CH₂—CH₂—V, with V being a halogen, in particular —F;
   —O—(C₁-C₄)-alkyl groups, in particular —OMe;
   —S—(C₁-C₄)-alkyl groups, in particular —SMe;
   —O—(C₂-C₄)-alkyl-OH and —S—(C₂-C₄)-alkyl-OH groups, in particular —O—(CH₂)ᵢ—OH, with i ranging from 2 to 4, i being more particularly 2, 3 or 4;
   —O(—(C₂-C₄)-alkyl-O)ₙ—H and —S(—(C₂-C₄)-alkyl-O)ₙ—H groups, in particular —O(—CH₂—CH₂—O)ₙ—H, with n ranging from 2 to 4, n being more particularly 2;
   —O—(C₂-C₄)-alkyl-O—(C₁-C₄)-alkyl, —S—(C₂-C₄)-alkyl-O—(C₁-C₄)-alkyl, —O—(C₂-C₄)-alkyl-S—(C₁-C₄)-alkyl and —S—(C₂-C₄)-alkyl-S—(C₁-C₄)-alkyl groups, in particular —O—(CH₂)ᵢ—OMe, with i ranging from 2 to 4, i being more particularly 2;
   —O—(C₂-C₄)-alkyl-O—(C₁-C₄)-alkyl-W″, —S—(C₂-C₄)-alkyl-O—(C₁-C₄)-alkyl-W″, —O—(C₂-C₄)-alkyl-S—(C₁-C₄)-alkyl-W″ and —S—(C₂-C₄)-alkyl-S—(C₁-C₄)-alkyl-W″ groups, with W″ being an halogen, in particular —F, for example —O—CH₂—CH₂—O—(CH₂)₂—F;
   —O(—(C₂-C₄)-alkyl-O)ₘ—(C₁-C₄)-alkyl and —S(—(C₂-C₄)-alkyl-O)ₘ—(C₁-C₄)-alkyl groups, in particular —O(—CH₂—CH₂—O)ₘ-Me or —O(—CH₂—CH₂—O)ₘ-Et, with m ranging from 2 to 4, m being more particularly 2;
   —O(—(C₂-C₄)-alkyl-O)ₘ—(C₁-C₄)-alkyl-W″—S(—(C₂-C₄)-alkyl-O)ₘ—(C₁-C₄)-alkyl-W″ groups, with W″ being an halogen, in particular —F, for example —O(—CH₂—CH₂—O)₂—(CH₂)₂—F;
   —O—(C₁-C₆)-alkyl-V and —S—(C₁-C₆)-alkyl-V groups, in particular —CH₂—CH₂—V; V being chosen from the group comprising:
      —C(═O)NR_{d}R_{d}′, R_{d} and R_{d}′ being independently H or a —(C₁-C₆)-alkyl group, in particular H;
      —S(═O)₂NR_{d}R_{d}′, R_{d} and R_{d}′ being independently H or a —(C₁-C₆)-alkyl group, in particular H;
      —C(═O)OR_{d}, with R_{d} being H or a —(C₁-C₆)-alkyl group;
      —S(═O)₂OH;
      halogens, in particular —F;
      -cyclo-(C₃-C₆)-alkyl groups being optionally substituted by at least one group chosen from —(C₁-C₆)-alkyl and ═O groups;
      -heterocyclo-(C₃-C₆)-alkyl groups being optionally substituted by at least one group chosen from —(C₁-C₆)-alkyl and ═O groups, for example imidazolidine-2,4-dionyl;
   said O—(C₁-C₆)-alkyl-V group being for example of the following formula:

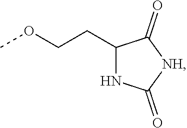

—O—(C₁-C₄)-alkyl-Ar, in particular —O—(CH₂)ⱼ-Ar, with j ranging from 1 to 4, j being more particularly 1 or 2, and Ar being an aryl, for example phenyl, or heteroaryl, for example indolyl, tetrazolyl, pyridinyl, pyrimidinyl, pyrazinyl or pyridazinyl, group being optionally substituted by at least one R″ group, in particular at least one —OH group,
R″ being chosen from the group comprising:
   —OH;
   halogens, in particular —F;
   a —O—(C₁-C₆)-alkyl and —S—(C₁-C₆)-alkyl, in particular a —OMe, or —O-cyclo-(C₃-C₆)-alkyl, in particular a —O-cyclopropyl;
   —O—(C₂-C₄)-alkyl-OH and —S—(C₂-C₄)-alkyl-OH groups, in particular —O—(CH₂)ᵢ—OH, with i ranging from 2 to 4, i being more particularly 2, 3 or 4;
   —O(—(C₂-C₄)-alkyl-O)ₙ—H and —S(—(C₂-C₄)-alkyl-O)ₙ—H groups, in particular —O(—CH₂—CH₂—O)ₙ—H, with n ranging from 2 to 4, n being more particularly 2;
   —O—(C₂-C₄)-alkyl-O—(C₁-C₄)-alkyl, —S—(C₂-C₄)-alkyl-O—(C₁-C₄)-alkyl, —O—(C₂-C₄)-alkyl-S—(C₁-C₄)-alkyl and —S—(C₂-C₄)-alkyl-S—(C₁-C₄)-alkyl groups, in particular —O—(CH₂)ᵢ—OMe, with i ranging from 2 to 4, i being more particularly 2;
   —O—(C₂-C₄)-alkyl-O—(C₁-C₄)-alkyl-W″, —S—(C₂-C₄)-alkyl-O—(C₁-C₄)-alkyl-W″, —O—(C₂-C₄)-alkyl-S—(C₁-C₄)-alkyl-W″ and —S—(C₂-C₄)-alkyl-S—(C₁-C₄)-alkyl-W″ groups, with W″ being an halogen, in particular —F, for example —O—CH₂—CH₂—O—(CH₂)₂—F; —O(—(C₂-C₄)-alkyl-O)ₘ—(C₁-C₄)-alkyl groups, in particular —O(—CH₂—CH₂—O)ₘ-Me or —O(—CH₂—CH₂—O)ₘ-Et, with m ranging from 2 to 4, m being more particularly 2;
   —O(—(C₂-C₄)-alkyl-O)ₘ—(C₁-C₄)-alkyl-W″ and —S(—(C₂-C₄)-alkyl-O)ₘ—(C₁-C₄)-alkyl-W″ groups, with W″ being an halogen, in particular —F, for example —O(—CH₂—CH₂—O)₂—(CH₂)₂—F;
   a —(C₁-C₆)-alkyl or -cyclo-(C₃-C₆)-alkyl, in particular a cyclopropyl;
   —(C₁-C₆)-alkyl-W groups, in particular —(CH₂)ₖ—W groups, said groups being optionally further substituted by at least a —W′ group, with k ranging from 1 to 6, k being more particularly 1 or 2, and W and W′ being an halogen, in particular —F, ORₐ, with Rₐ representing —H or —(C₁-C₄)-alkyl, in particular H or Me, or a PEG group, in particular of following formula —(CH₂—CH₂—O)ₗ—H, with l ranging from 1 to 10, in particular from 2 to 4, for example 2; or W and W′ being NR_{b}H, with R_{b} representing —H, a —(C₁-C₄)-alkyl group, or a —(C₁-C₄)-alkyl-Ar′, with Ar′ being an aryl or heteroaryl group, in particular a furanyl;
   —(C₁-C₆)-alkyl-Y—Z groups, in particular —(CH₂)ₗ—Y—Z, with l ranging from 1 to 6, 1 being more particularly 1 or 2, Y being —O—, —NH— or being absent, and Z being chosen from saturated heterocyclic groups, in particular morpholinyl, tetrahydropyranyl, imidazolidinyl, said saturated heterocyclic groups being optionally substituted by at least one ═O group, representing for example imidazolidine-2,4-dione; and heteroaryl groups, in particular tetrazolyl;
   an aryl or heteroaryl group, in particular a pyrazolyl or a tetrazolyl, said group being optionally substituted by at least one R_{c} group chosen from —(C₁-C₆)-alkyl and —NH₂ groups;
   a —(C₀-C₆)-alkyl-C(═O)OR_{d}, with R_{d} being H or a —(C₁-C₆)-alkyl group, in particular H, —(C₀-C₆)-alkyl-C(═O)NR_{d}R_{d}′, R_{d} and R_{d}′ being independently H or a —(C₁-C₆)-alkyl group, in particular H, —(C₀-C₆)-alkyl-S(═O)₂OR_{d}, R_{d} being H or a —(C₁-C₆)-alkyl group, in particular H, or —(C₀-C₆)-alkyl-S(═O)₂NR_{d}R_{d}′ group, R_{d} and R_{d}′ being independently H, a —(C₁-C₆)-alkyl group, in particular H, or a —NH—C(═O)—C₁-C₆-alkyl group, in particular —NH—C(═O)-Me group, more particularly a —COOH, —C(═O)NH₂, —S(═O)₂OH, —S(═O)₂NH₂ or —(CH₂)₂—S(═O)₂NH₂ group;
   a —NHC(═O)—(C₁-C₆)-alkyl, in particular —NHC(═O)-Me;
said —O—(C₁-C₄)-alkyl-Ar group being for example of one of the following formulae:

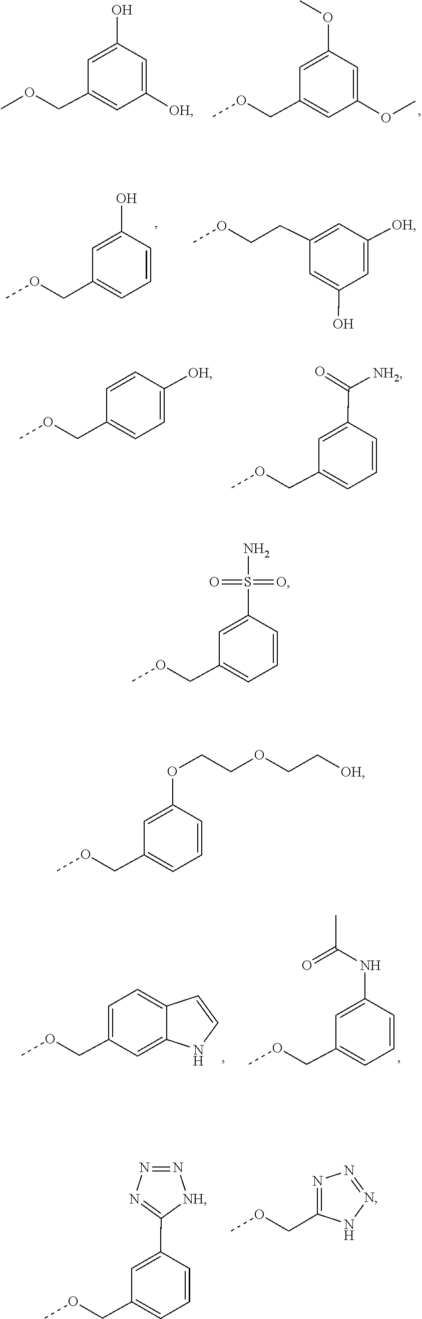

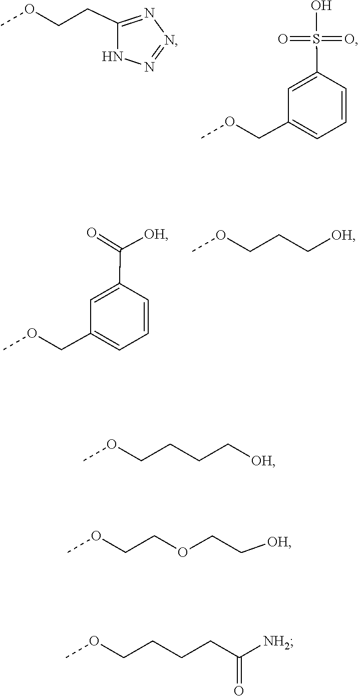

Halogens, in particular —F;
—CF₃;
or R is —F and its geminal hydrogen is replaced by —F, R forming with the carbon atom C to which it is bound and its —F replaced geminal hydrogen a CF₂ residue;
or an enantiomer, diastereoisomer, and/or a pharmaceutically acceptable salt or solvate thereof.

The group —F₂ as used in the present invention, refers to two —F atoms bound to the same carbon atom C.

In a particular embodiment, R is chosen from —O—(C₁-C₄)-alkyl-Ar groups, in particular —O—(CH₂)ⱼ-Ar, with j ranging from 1 to 4, j being more particularly 1, and Ar being an aryl group being optionally substituted by at least one R″ group as defined above.

In a particular embodiment, said Ar group is substituted by one R″ group as defined above.

In another particular embodiment, said Ar group is substituted by two R″ groups independently chosen as defined above.

In a particular embodiment, said Ar group is substituted by one —OH group and by one R″ group as defined above.

In a more particular embodiment, said Ar group is chosen from:

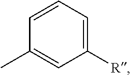

R″ being as defined above,

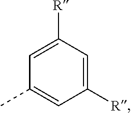

both R″ groups being independently chosen as mentioned above,

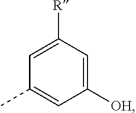

R″ groups being chosen as mentioned above,

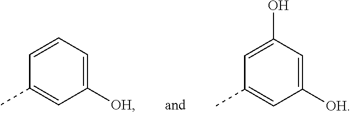

In a particular embodiment, R is chosen from:

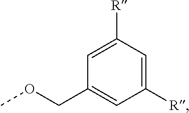

both R″ groups being independently chosen as mentioned above,

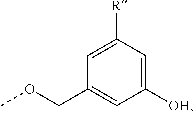

R″ groups being chosen as mentioned above,

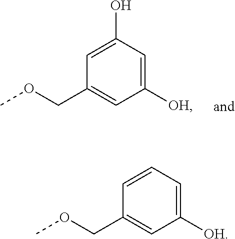

For the purpose of the invention, the term “pharmaceutically acceptable” is intended to mean what is useful to the preparation of a pharmaceutical composition, and what is generally safe and non-toxic, for a pharmaceutical use.

The term “pharmaceutically acceptable salt or solvate” is intended to mean, in the framework of the present invention, a salt or solvate of a compound which is pharmaceutically acceptable, as defined above, and which possesses the pharmacological activity of the corresponding compound.

The pharmaceutically acceptable salts comprise:
(1) acid addition salts formed with inorganic acids such as hydrochloric, hydrobromic, sulfuric, nitric and phosphoric acid and the like; or formed with organic acids such as acetic, benzenesulfonic, fumaric, glucoheptonic, gluconic, glutamic, glycolic, hydroxynaphtoic, 2-hydroxyethanesulfonic, lactic, maleic, malic, mandelic, methanesulfonic, muconic, 2-naphtalenesulfonic, propionic, succinic, dibenzoyl-L-tartaric, tartaric, p-toluenesulfonic, trimethylacetic, and trifluoroacetic acid and the like, and
(2) base addition salts formed when an acid proton present in the compound is either replaced by a metal ion, such as an alkali metal ion, an alkaline-earth metal ion, or an aluminium ion; or coordinated with an organic or inorganic base. Acceptable organic bases comprise diethanolamine, ethanolamine, N-methylglucamine, triethanolamine, tromethamine and the like. Acceptable inorganic bases comprise aluminium hydroxide, calcium hydroxide, potassium hydroxide, sodium carbonate and sodium hydroxide.

Acceptable solvates for the therapeutic use of the compounds of the present invention include conventional solvates such as those formed during the last step of the preparation of the compounds of the invention due to the presence of solvents. As an example, mention may be made of solvates due to the presence of water (these solvates are also called hydrates) or ethanol.

It is recognized that compounds of the present invention may exist in various stereoisomeric forms. As such, the compounds of the present invention include both diastereomers and enantiomers. The compounds are normally prepared as racemates and can conveniently be used as such, but individual enantiomers can be isolated or synthesized by conventional techniques if so desired. Such racemates and individual enantiomers and mixtures thereof form part of the present invention.

It is well known in the art how to prepare and isolate such optically active forms. Specific stereoisomers can be prepared by stereospecific synthesis using enantiomerically pure or enantiomerically enriched starting materials. The specific stereoisomers of either starting materials or products can be resolved and recovered by techniques known in the art, such as resolution of racemic forms, normal, reverse-phase, and chiral chromatography, recrystallization, enzymatic resolution, or fractional recrystallization of addition salts formed by reagents used for that purpose. Useful methods of resolving and recovering specific stereoisomers described in Eliel, E. L.; Wilen, S. H. Stereochemistry of Organic Compounds; Wiley: New York, 1994, and Jacques, J, et al. Enantiomers, Racemates, and Resolutions; Wiley: New York, 1981, each incorporated by reference herein in their entireties.

In a particular embodiment, the present invention relates to a compound as defined above, of following formula (I):

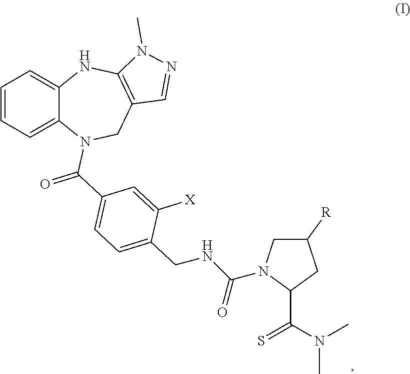

wherein:
X is chosen from —H, halogens, —(C₁-C₆)-alkyl groups, —O—(C₁-C₆)-alkyl groups and —O—CF₃, in particular -Me or —Cl, more particularly -Me;
R is chosen from the group comprising:
   —O—(C₁-C₄)-alkyl groups, in particular —OMe;
   —O—(C₂-C₄)-alkyl-OH groups, in particular —O—(CH₂)ᵢ—OH, with i ranging from 2 to 4, i being more particularly 2;
   —O(—(C₂-C₄)-alkyl-O)ₙ—H groups, in particular —O(—CH₂—CH₂)ₙ—OH, with n ranging from 2 to 4, n being more particularly 2;
   —O—(C₂-C₄)-alkyl-O—(C₁-C₄)-alkyl groups, in particular —O—(CH₂)ᵢ—OMe, with i ranging from 2 to 4, i being more particularly 2;
   —O(—(C₂-C₄)-alkyl-O)ₘ—(C₁-C₄)-alkyl groups, in particular —O(—CH₂—CH₂—O)ₘ-Me or —O(—CH₂—CH₂—O)ₘ-Et, with m ranging from 2 to 4, m being more particularly 2;
   —O—(C₁-C₄)-alkyl-Ar, in particular —O—(CH₂)ⱼ-Ar, with j ranging from 1 to 4, j being more particularly 1, and Ar being an aryl group being optionally substituted by at least one R″ group, in particular at least one —OH group, said —O—(C₁-C₄)-alkyl-Ar group being for example of the following formula:

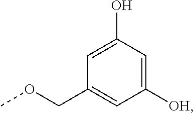

R″ being chosen from the group comprising:
   —OH;
   a —O—(C₁-C₆)-alkyl or —O-cyclo-(C₃-C₆)-alkyl, in particular a —O-cyclopropyl;
   a —(C₁-C₆)-alkyl or -cyclo-(C₃-C₆)-alkyl, in particular a cyclopropyl;
   —(C₁-C₆)-alkyl-W groups, in particular —(CH₂)ₖ—W groups, said groups being optionally further substituted by at least a —W′ group, with k ranging from 1 to 6, k being more particularly 1 or 2, and W and W′ being independently ORₐ, with Rₐ representing —H or —(C₁-C₄)-alkyl, in particular H or Me, or a PEG group, in particular of following formula —(CH₂—CH₂—O)₁—H, with l ranging from 1 to 10, in particular from 2 to 4, for example 2; or W and W′ being NR_{b}H, with R_{b} representing —H, a —(C₁-C₄)-alkyl group, or a —(C₁-C₄)-alkyl-Ar′, with Ar′ being an aryl or heteroaryl group, in particular a furanyl;
   —(C₁-C₆)-alkyl-Y—Z groups, in particular —(CH₂)ₗ—Y—Z, with l ranging from 1 to 6, 1 being more particularly 1 or 2, Y being —O—, —NH— or being absent, and Z being chosen from saturated heterocyclic groups, in particular morpholinyl, tetrahydropyranyl, imidazolidinyl, said saturated heterocyclic groups being optionally substituted by at least one ═O group, representing for example imidazolidine-2,4-dione; and heteroaryl groups, in particular tetrazolyl;
   An aryl or heteroaryl group, in particular a pyrazolyl, said group being optionally substituted by at least one R_{c} group chosen from —(C₁-C₆)-alkyl and —NH₂ groups;
   a —(C₀-C₆)-alkyl-C(═O)NH₂ or —(C₀-C₆)-alkyl-S(═O)₂NH₂ group, in particular a —C(═O)NH₂ or —(CH₂)₂—S(═O)₂NH₂ group;
Halogens, in particular —F.

In a particular embodiment, the present invention relates to a compound as defined above, of following formula (I):

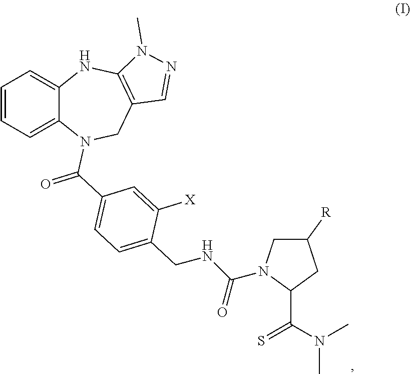

wherein:
X is chosen from —H, halogens, —(C₁-C₆)-alkyl groups, —O—(C₁-C₆)-alkyl groups and —O—CF₃, in particular -Me or —Cl, more particularly -Me;
R is chosen from the group comprising:
   —O—(C₁-C₄)-alkyl groups, in particular —OMe;
   —O—(C₂-C₄)-alkyl-OH groups, in particular —O—(CH₂)ᵢ—OH, with i ranging from 2 to 4, i being more particularly 2;
   —O—(C₂-C₄)-alkyl-O—(C₁-C₄)-alkyl groups, in particular —O—(CH₂)ᵢ—OMe, with i ranging from 2 to 4, i being more particularly 2;
   —O(—(C₂-C₄)-alkyl-O)ₘ—(C₁-C₄)-alkyl groups, in particular —O(—CH₂—CH₂—O)ₘ-Me or —O(—CH₂—CH₂—O)ₘ-Et, with m ranging from 2 to 4, m being more particularly 2;
   —O—(C₁-C₆)-alkyl-V and —S—(C₁-C₆)-alkyl-V groups, in particular —CH₂—CH₂—V; V being chosen from the group comprising:
      -cyclo-(C₃-C₆)-alkyl groups being optionally substituted by at least one group chosen from —(C₁-C₆)-alkyl and ═O groups;
      -heterocyclo-(C₃-C₆)-alkyl groups being optionally substituted by at least one group chosen from —(C₁-C₆)-alkyl and ═O groups, for example imidazolidine-2,4-dionyl;
   said O—(C₁-C₆)-alkyl-V group being for example of the following formula:

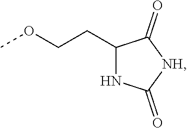

—O—(C₁-C₄)-alkyl-Ar, in particular —O—(CH₂)ⱼ-Ar, with j ranging from 1 to 4, j being more particularly 1, and Ar being an aryl group being optionally substituted by at least one R″ group, in particular at least one —OH group, said —O—(C₁-C₄)-alkyl-Ar group being for example of the following formula:

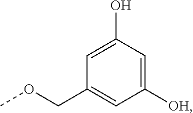

—O—(CH₂)-Ar, with Ar being an heteroaryl, for example indolyl, tetrazolyl, pyridinyl, pyrimidinyl, pyrazinyl or pyridazinyl, said Ar group being optionally substituted by at least one R″ group, in particular at least one —OH group,
R″ being chosen from the group comprising:
   —OH;
   a —O—(C₁-C₆)-alkyl or —O-cyclo-(C₃-C₆)-alkyl, in particular a —O-cyclopropyl;
   a —(C₁-C₆)-alkyl or -cyclo-(C₃-C₆)-alkyl, in particular a cyclopropyl;
   —(C₁-C₆)-alkyl-W groups, in particular —(CH₂)ₖ—W groups, said groups being optionally further substituted by at least a —W′ group, with k ranging from 1 to 6, k being more particularly 1 or 2, and W and W′ being independently ORₐ, with Rₐ representing —H or —(C₁-C₄)-alkyl, in particular H or Me; or W and W′ being NR_{b}H, with R_{b} representing —H, a —(C₁-C₄)-alkyl group, or a —(C₁-C₄)-alkyl-Ar′, with Ar′ being an aryl or heteroaryl group, in particular a furanyl;
   —(C₁-C₆)-alkyl-Y—Z groups, in particular —(CH₂)ₗ—Y—Z, with l ranging from 1 to 6, 1 being more particularly 1 or 2, Y being —O—, —NH— or being absent, and Z being chosen from saturated heterocyclic groups, in particular morpholinyl, tetrahydropyranyl, imidazolidinyl, said saturated heterocyclic groups being optionally substituted by at least one ═O group, representing for example imidazolidine-2,4-dione; and heteroaryl groups, in particular tetrazolyl;
   a —(C₀-C₆)-alkyl-S(═O)₂NH₂ group, in particular a-(CH₂)₂—S(═O)₂NH₂ group; said Ar group not being substituted by only one —OH group, or by two —O—(C₁-C₆)-alkyl or —O-cyclo-(C₃-C₆)-alkyl groups;
Halogens, in particular —F;

In another particular embodiment, the present invention relates to a compound as defined above, of following formula (I):

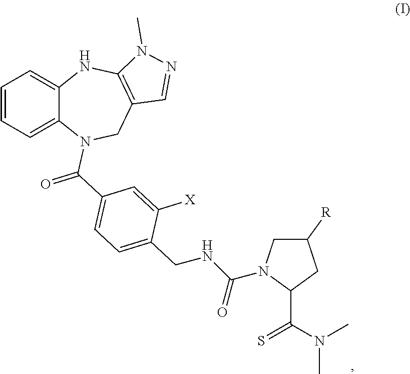

wherein:
X is chosen from —H, halogens, —(C₁-C₆)-alkyl groups, —O—(C₁-C₆)-alkyl groups and —O—CF₃, in particular -Me or —Cl, more particularly -Me;
R is chosen from —O—(C₁-C₄)-alkyl-Ar groups, in particular —O—(CH₂)ⱼ-Ar, with j ranging from 1 to 4, j being more particularly 1, and Ar being optionally an aryl group substituted by at least one R″ group, in particular at least one —OH group, said —O—(C₁-C₄)-alkyl-Ar group being for example of the following formula:

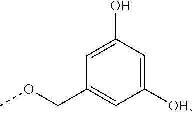

R″ being chosen from the group comprising:
   —OH;
   a —O—(C₁-C₆)-alkyl or —O-cyclo-(C₃-C₆)-alkyl, in particular a —O-cyclopropyl;
   a —(C₁-C₆)-alkyl or -cyclo-(C₃-C₆)-alkyl, in particular a cyclopropyl;
   —(C₁-C₆)-alkyl-W groups, in particular —(CH₂)ₖ—W groups, said groups being optionally further substituted by at least a —W′ group, with k ranging from 1 to 6, k being more particularly 1 or 2, and W and W′ being ORₐ, with Rₐ representing —H or —(C₁-C₄)-alkyl, in particular H or Me, or a PEG group, in particular of following formula —(CH₂—CH₂—O)₁—H, with l ranging from 1 to 10, in particular from 2 to 4, for example 2; or W and W′ being NR_{b}H, with R_{b} representing —H, a —(C₁-C₄)-alkyl group, or a —(C₁-C₄)-alkyl-Ar′, with Ar′ being an aryl or heteroaryl group, in particular a furanyl;
   —(C₁-C₆)-alkyl-Y—Z groups, in particular —(CH₂)ₗ—Y—Z, with l ranging from 1 to 6, 1 being more particularly 1 or 2, Y being —O—, —NH— or being absent, and Z being chosen from saturated heterocyclic groups, in particular morpholinyl, tetrahydropyranyl, imidazolidinyl, said saturated heterocyclic groups being optionally substituted by at least one ═O group, representing for example imidazolidine-2,4-dione; and heteroaryl groups, in particular tetrazolyl;
   An aryl or heteroaryl group, in particular a pyrazolyl, said group being optionally substituted by at least one R_{c} group chosen from —(C₁-C₆)-alkyl and —NH₂ groups;
   a —(C₀-C₆)-alkyl-C(═O)NH₂ or —(C₀-C₆)-alkyl-S(═O)₂NH₂ group, in particular a —C(═O)NH₂ or —(CH₂)₂—S(═O)₂NH₂ group.

In a particular embodiment, the present invention relates to a compound as defined above, of one of the following formulae:

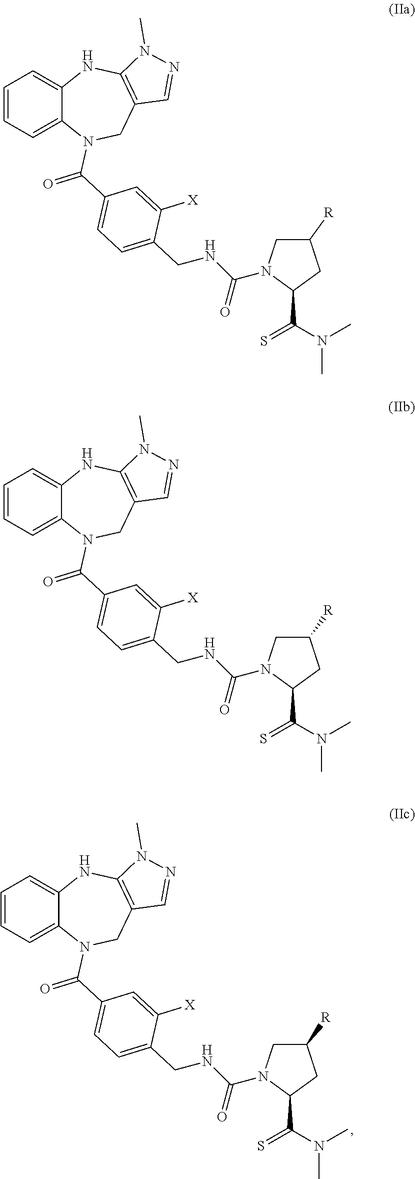

wherein X and R are as defined above.

In a particular embodiment, X is Me.

In another particular embodiment, X is Cl.

In a particular embodiment, R is not a —O—(C₁-C₄)-alkyl group, in particular not —OMe.

In a particular embodiment, the present invention relates to a compound as defined above, wherein R is chosen from:

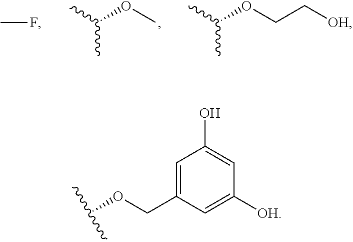

In a particular embodiment, the present invention relates to a compound as defined above, wherein R is chosen from:

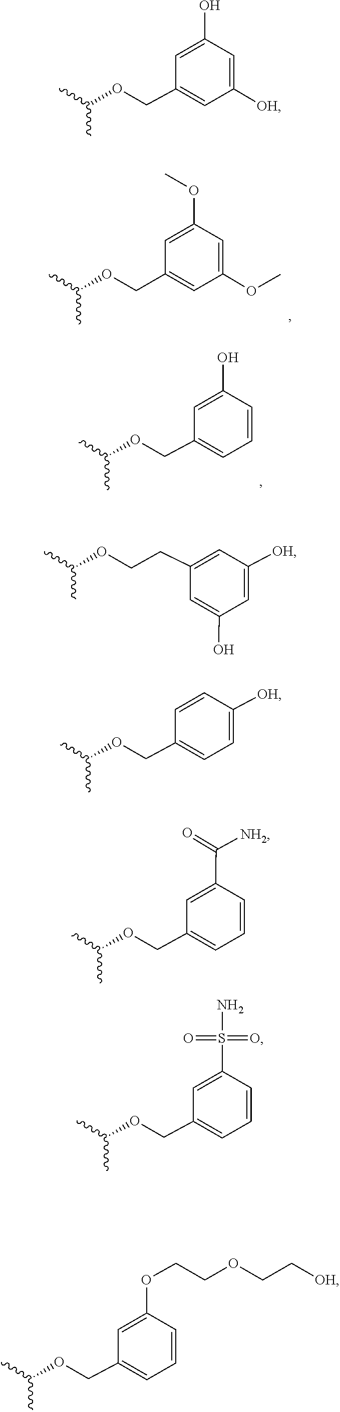

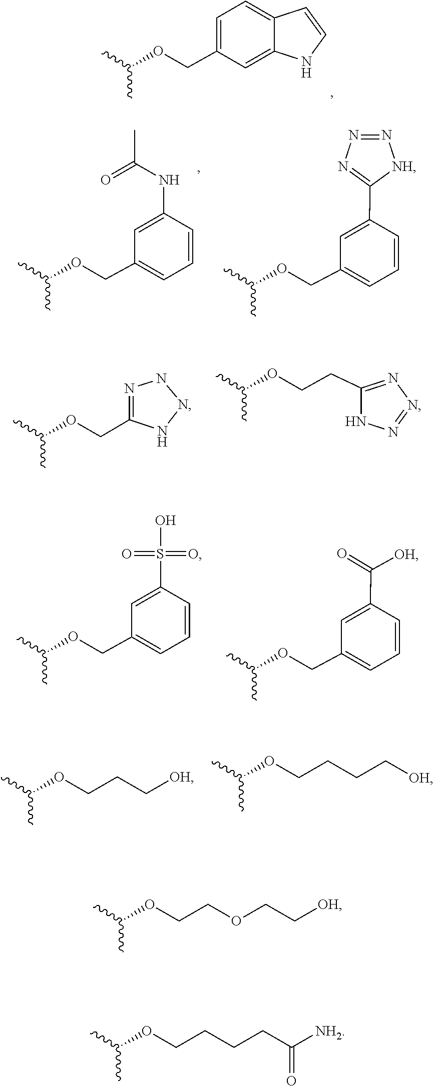

In another aspect, the present invention also relates to a pharmaceutical composition comprising a compound as defined above in admixture with at least one pharmaceutical acceptable excipient.

All the embodiments described above in relation with the compound of the invention apply here as well, alone or in any combination.

The compound or the pharmaceutical composition of the present invention may be administered in the form of a conventional pharmaceutical composition by any route including orally, intramuscularly, subcutaneously, topically, intranasally, intraperitoneally, intrathoracially, intravenously, epidurally, intrathecally, intracerebroventricularly and by injection into the joints, in particular orally, intravenously or intranasally.

The dosage will depend on the route of administration, the severity of the disease, age and weight of the patient and other factors normally considered by the attending physician, when determining the individual regimen and dosage level at the most appropriate for a particular patient.

For preparing pharmaceutical compositions from the compounds of the present invention, inert, pharmaceutically acceptable carriers can be either solid or liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or tablet disintegrating agents; it can also be an encapsulating material.

Tablets, powders, cachets, and capsules can be used as solid dosage forms suitable for oral administration.

Liquid form compositions include solutions, suspensions, and emulsions. For example, sterile water or propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavoring agents, stabilizers, and thickening agents as desired. Aqueous solutions for oral use can be made by dispersing the finely divided active component in water together with a viscous material such as natural synthetic gums, resins, methyl cellulose, sodium carboxymethyl cellulose, and other suspending agents known to the pharmaceutical formulation art.

Compositions comprising microspheres based on polymers such as hyaluronic acid, dextran and/or starch can for example be used for intranasal administration.

Depending on the mode of administration, the pharmaceutical composition will according to one embodiment of the present invention include 0.05% to 99% weight (percent by weight), according to an alternative embodiment from 0.10 to 50% weight, of the compound of the present invention, all percentages by weight being based on total composition. A therapeutically effective amount for the practice of the present invention may be determined, by the use of known criteria including the age, weight and response of the individual patient, and interpreted within the context of the disease which is being treated or which is being prevented, by one of ordinary skills in the art.

In another aspect, the present invention also relates to a dermatologic and/or cosmetic composition comprising a compound as defined above in admixture with at least one pharmaceutical acceptable excipient.

All the embodiments described above in relation with the compound of the invention apply here as well, alone or in any combination.

The dermatologic and/or cosmetic composition of the present invention may be administered in the form of a conventional dermatologic and/or cosmetic composition by any route including orally, topically, in particular topically.

The dosage will depend on the route of administration, the severity of the disease, condition or disorder, age and weight of the patient and other factors normally considered by the attending physician or practitioner, when determining the individual regimen and dosage level at the most appropriate for a particular patient.

For preparing dermatologic and/or cosmetic compositions from the compounds of the present invention, inert, pharmaceutically acceptable carriers can be either solid or liquid.

The dermatologic and/or cosmetic comprising composition may be in any suitable form commonly used in cosmetics. In particular embodiments, the composition is selected from a solution, a suspension, a cream, a lotion, a paste, an emulsion, a gel, a foundation, a serum, and an ointment.

Depending on the mode of administration, the dermatologic and/or cosmetic composition will according to one embodiment of the present invention include 0.05% to 99% weight (percent by weight), according to an alternative embodiment from 0.10 to 50% weight, of the compound of the present invention, all percentages by weight being based on total composition.

In another aspect, the present invention also relates to a compound as defined above for use in the prevention and/or treatment of a disease, condition or disorder, which is autism, autism spectrum disorders, in particular for the treatment of social interaction impairments in autism spectrum disorders, pain, drug and/or alcohol addiction, depression, post-traumatic stress, anxiety, eating disorders, notably anorexia, bulimia and Prader-Willi syndrome, schizophrenia, neurodevelopmental disorders, social interaction disorders, borderline behavior, ageing of the skin, muscles, bones, erectile dysfunction, and any disease for which oxytocin is known to have a beneficial effect.

All the embodiments described above in relation with the compound of the invention apply here as well, alone or in any combination.

In a particular embodiment, the present invention relates to a compound as defined above for use in the prevention and/or treatment of a disease, condition or disorder, which is autism, autism spectrum disorders, in particular for the treatment of social interaction impairments in autism spectrum disorders.

In another aspect, the present invention also relates to the use of a compound as defined above to relieve or reduce pain. All the embodiments described above in relation with the compound of the invention apply here as well, alone or in any combination.

In another aspect, the present invention also relates to the use of a compound as defined above to reduce ageing of the skin, muscles, bones, in particular of the skin.

All the embodiments described above in relation with the compound of the invention apply here as well, alone or in any combination.

In another aspect, the present invention also relates to the use of a compound as defined above to facilitate erection, lactation, childbirth, breastfeeding, and/or post-partum period.

All the embodiments described above in relation with the compound of the invention apply here as well, alone or in any combination.

In another aspect, the invention also relates to a method of prevention and/or treatment of a disease, condition or disorder, which is autism, autism spectrum disorders, in particular for the treatment of social interaction impairments in autism spectrum disorders, pain, drug and/or alcohol addiction, depression, post-traumatic stress, anxiety, eating disorders, notably anorexia, bulimia and Prader-Willi syndrome, schizophrenia, neurodevelopmental disorders, social interaction disorders, borderline behavior, ageing of the skin, muscles, bones, erectile dysfunction, and any disease for which oxytocin is known to have a beneficial effect, comprising administering to a subject in need thereof a compound as defined above.

In another aspect, the invention also relates to a method to reduce ageing of the skin, muscles, bones, in particular of the skin, or to facilitate erection, lactation, childbirth, breastfeeding, and/or post-partum period, comprising administering to a subject in need thereof a compound as defined above.

In another aspect, the invention also relates to a composition comprising at least one compound as defined above and a diuretic, for example urea, or its salt, or a V2r antagonist, in particular a vaptan such as tolvaptan, lixivaptan, or satavaptan, as a combination product for simultaneous, separate or spread therapeutic use in the prevention and/or treatment of a disease, condition or disorder, which is autism, autism spectrum disorders, in particular for the treatment of social interaction impairments in autism spectrum disorders, pain, drug and/or alcohol addiction, depression, post-traumatic stress, anxiety, eating disorders, notably anorexia, bulimia and Prader-Willi syndrome, schizophrenia, neurodevelopmental disorders, social interaction disorders, borderline behavior, ageing of the skin, muscles, bones, erectile dysfunction, and any disease for which oxytocin is known to have a beneficial effect. In another aspect, the invention also relates to a composition comprising at least one compound as defined above and a diuretic, for example urea, or its salt, or a V2r antagonist, in particular a vaptan such as tolvaptan, lixivaptan, or satavaptan, as a combination product for simultaneous, separate or spread therapeutic use to reduce ageing of the skin, muscles, bones, in particular of the skin, or to facilitate erection, lactation, childbirth, breastfeeding, and/or post-partum period.

### Definitions

The following terms and expressions contained herein are defined as follows:

As used herein, a range of values in the form “x-y” or “x to y”, or “x through y”, include integers x, y, and the integers therebetween. For example, the phrases “1-6”, or “1 to 6” or “1 through 6” are intended to include the integers 1, 2, 3, 4, 5, and 6. Preferred embodiments include each individual integer in the range, as well as any subcombination of integers. For example, preferred integers for “1-6” can include 1, 2, 3, 4, 5, 6, 1-2, 1-3, 1-4, 1-5, 2-3, 2-4, 2-5, 2-6, etc.

The term “—(C₁-C₆)alkyl”, as used in the present invention, refers to a straight or branched saturated hydrocarbon chain containing from 1 to 6 carbon atoms including, but not limited to, methyl, ethyl, n-propyl, iso-propyl, n-butyl, iso-butyl, sec-butyl, t-butyl, n-pentyl, n-hexyl, and the like.

The term “aryl”, as used in the present invention, and which may be abbreviated “Ar”, refers in particular to an aromatic hydrocarbon group comprising preferably 6 to 10 carbon atoms and comprising one or more, notably 1 or 2, fused rings, such as, for example, a phenyl or naphtyl group. Advantageously, it will be a phenyl group.

The term “—(C₁-C₄)-alkyl-aryl”, as used in the present invention, refers to aryl group as defined above bound to the molecule via a (C₁-C₄)alkyl group as defined above.

The term “—(C₃-C₆)-cycloalkyl” as used in the present invention refers to a saturated hydrocarbon monocycle having 3-6 carbon atom members.

The term “saturated heterocyclic group” as used in the present invention refers in particular to a saturated hydrocarbon monocycle or polycycle (comprising fused, bridged or spiro rings), such as a bicycle, in which one or more, advantageously 1 to 4, and more advantageously 1 or 2, carbon atoms have each been replaced with a heteroatom selected from nitrogen, oxygen and sulphur atoms, and notably being a nitrogen atom. Advantageously, the heterocycle comprises 5 to 15, notably 5 to 10 atoms in the ring(s). The ring(s) of the heterocycle has/have advantageously 5 or 6 members.

According to a particular embodiment, the saturated heterocyclic group is in particular a saturated, hydrocarbon monocycle or bicycle (comprising fused, bridged or spiro rings, notably fused rings), each cycle having 5 or 6 members and 1 to 4, notably 1 or 2, carbon atoms having each been replaced with a nitrogen or oxygen atom, notably a nitrogen atom.

The term “heteroaryl” as used in the present invention refers in particular to an aromatic hydrocarbon monocycle or bicycle (i.e. comprising fused rings), each cycle having 5 or 6 members, notably 6 members, and 1 to 4, notably 1 or 2, carbon atoms having each been replaced with a nitrogen or oxygen atom, notably a nitrogen atom.

A heteroaryl can be notably thiophene, furan, pyrrole, imidazole, pyrazole, oxazole, isoxazole, thiazole, isothiazole, triazoles (1,2,3-triazole and 1,2,4-triazole), benzofuran, indole, benzothiophene, benzimidazole, indazole, benzoxazole, benzisoxazole, benzothiazole, benzisothiazole, pyridine, pyrimidine, pyridazine, pyrazine, triazine, quinoline, isoquinoline, quinoxaline, quinazoline, etc.

The term “—(C₁-C₄)alkyl-heteroaryl”, as used in the present invention, refers to aryl group as defined above bound to the molecule via a (C₁-C₄)alkyl group as defined above.

The term “halogen”, as used in the present invention, refers to a fluorine, bromine, chlorine or iodine atom.

### Synthesis

The compounds of the present invention may be prepared in a number of methods well known to those skilled in the art, including, but not limited to those described below, or through modifications of these methods by applying standard techniques known to those skilled in the art of organic synthesis. The appropriate modifications and substitutions will be readily apparent and well known or readily obtainable from the scientific literature to those skilled in the art. In particular, such methods can be found in R. C. Larock, Comprehensive Organic Transformations, Wiley-VCH Publishers, 2018.

All processes disclosed in association with the present invention are contemplated to be practiced on any scale, including milligram, gram, multigram, kilogram, multikilogram or commercial industrial scale.

It will be appreciated that the compounds of the present invention may contain one or more asymmetrically substituted carbon atoms, and may be isolated in optically active or racemic forms. Thus, all chiral, diastereomeric, racemic forms, isomeric forms of a structure are intended, unless the specific stereochemistry or isomeric form is specifically indicated. It is well-known in the art how to prepare and isolate such optically active forms. For example, mixtures of stereoisomers may be separated by standard techniques including, but not limited to, resolution of racemic forms, normal, reverse-phase, and chiral chromatography, preferential salt formation, recrystallization, and the like, or by chiral synthesis either from chiral starting materials or by deliberate synthesis of target chiral centers.

Compounds of the present invention may be prepared by a variety of synthetic routes. The reagents and starting materials are commercially available, or readily synthesized by well-known techniques by one of ordinary skill in the arts. All substituents, unless otherwise indicated, are as previously defined.

In the reactions described hereinafter, it may be necessary to protect reactive functional groups, for example hydroxy, amino, imino, thio or carboxy groups, where these are desired in the final product, to avoid their unwanted participation in the reactions. Conventional protecting groups may be used in accordance with standard practice, for examples see T. W. Greene and P. G. M. Wuts in Protective Groups in Organic Chemistry, 3^{rd} ed., John Wiley and Sons, 1999; J. F. W. McOmie in Protective Groups in Organic Chemistry, Plenum Press, 1973.

The reagents and starting materials are commercially available, or readily synthesized by well-known techniques by one of ordinary skill in the arts.

In particular, the compounds defined above are obtained according to the following procedure:

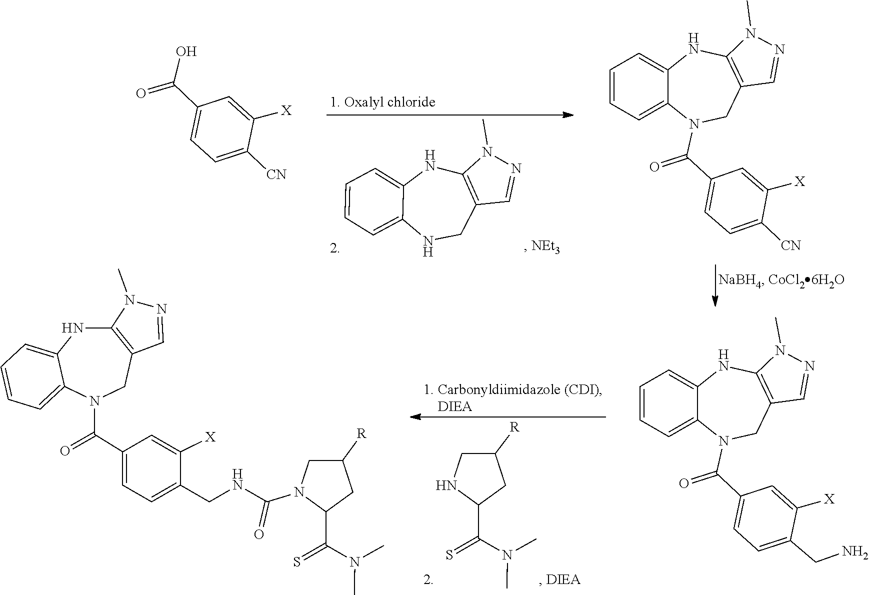

wherein X and R are as described above, or being protected by an ad hoc protecting group.

### EXAMPLES

### Example 1: Synthesis of the Compounds of the Invention

Compounds of the invention have been obtained as follows:

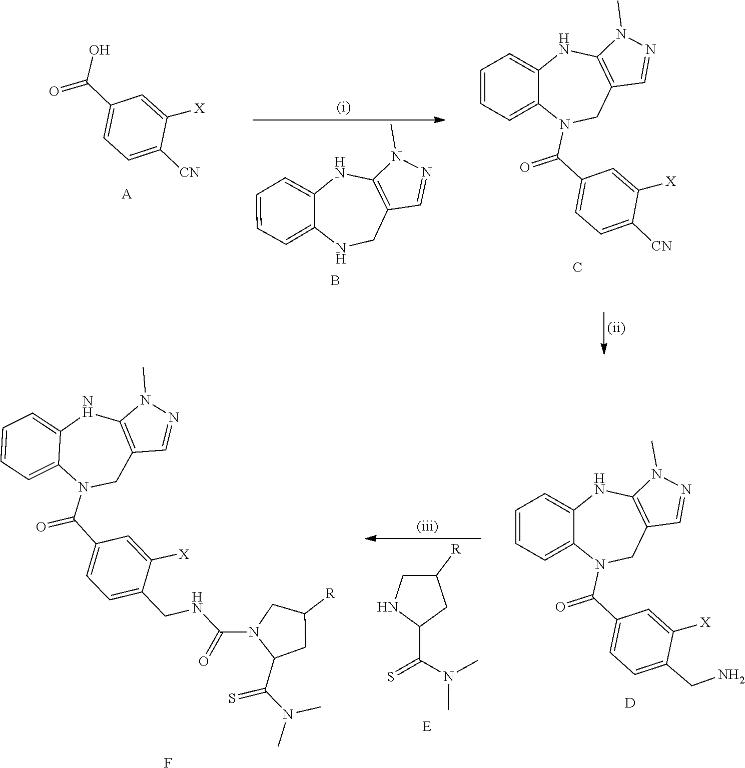

wherein X and R are as described above, or being protected by an ad hoc protecting group.

### Step (i)

To a solution of compound A (2 eq.) in anhydrous CH₂Cl₂, and DMF (a few drops), was added dropwise oxalyl chloride (4 eq.). The mixture was stirred for 15 minutes at 0° C., 2 hours at RT, concentrated under reduced pressure and dried under vacuum for 1 hour. The residue was dissolved in CH₂Cl₂, and added dropwise at 0° C. to a solution of compound B (1 eq.) and triethylamine (2 eq.) in anhydrous CH₂Cl₂. The obtained mixture was stirred for 30 minutes at 0° C. then overnight at RT. The crude product was dissolved in CH₂Cl₂ and the organic phase was washed with saturated KHSO₄. The aqueous phase was extracted with a CHCl₃/PrOH 8/2 v/v mixture. The combined organic phases were washed with saturated NaHCO₃, dried over Na₂SO₄, and evaporated under reduced pressure. Purification by silica gel chromatography (CH₂Cl₂/MeOH, 95/5 v/v) gave product C.

### Step (ii)

To a solution of compound C (1 eq.) in anhydrous methanol at 0° C., were added portionwise over 10 minutes cobalt chloride hexahydrate (2 eq.), and sodium borohydride (10 eq.). The obtained black mixture was stirred for 10 minutes at 0° C., then 1 hour at RT. The mixture was neutralized to pH 7-8 with a 1M KHSO₄ solution. Methanol was evaporated under reduced pressure. The residue was dissolved in 1M KHSO₄. The formed precipitate was filtered and washed with diethyl ether. The aqueous phase was extracted with diethyl ether and then basified to pH>10 with a 10M NaOH solution. The aqueous phase was again extracted with diethyl ether. The combined organic phases were dried over Na₂SO₄ and evaporated under reduced pressure to yield compound D.

### Step (iii)

A solution of compound D (1 eq.), of carbonyldiimidazole (1.2 eq) and of DIEA (1.5 eq) in DMF was stirred for 3 hours at RT. To this mixture was added a solution of compound E and DIEA (2.5 eq) in DMF. The reactional mixture was stirred overnight at RT and then evaporated under reduced pressure. The residue was purified by semi-preparative HPLC to obtain expected product F.

The following analytical data have been obtained:

| 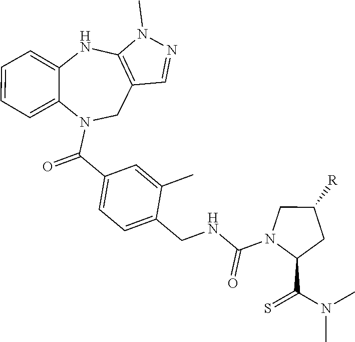 | | m/z (determined by LC-MS) [M + H]⁺. |
|---|---|---|
| Compound | R | |
| LIT-003-01 | 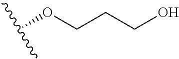 | 606.2 |
| LIT-003-02 | 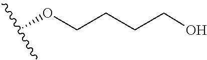 | 620.2 |
| LIT-003-03 | 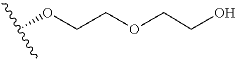 | 636.2 |
| LIT-003-04 | 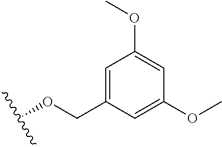 | 698.2 |
| LIT-003-05 | 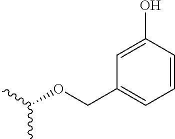 | 653.0 |
| LIT-003-07 | 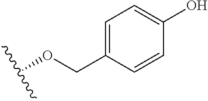 | 654.4 |
| LIT-003-08 | 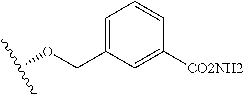 | 681.2 |
| LIT-003-09 | 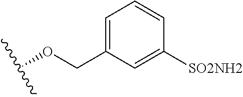 | 717.0 |
| LIT-003-10 | 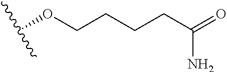 | 647.2 |
| LIT-003-11 | 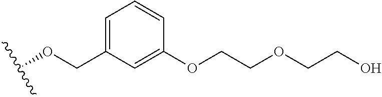 | 743.2 |
| LIT-003-12 | 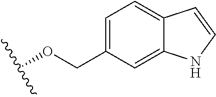 | 677.4 |
| LIT-003-13 | 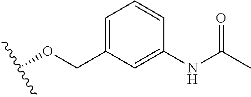 | 694.4 |
| LIT-003-14 | 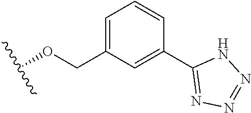 | 706.2 |
| LIT-003-15 | 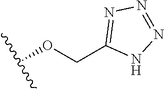 | 630.4 |
| LIT-003-16 | 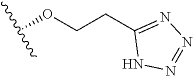 | 644.4 |
| LIT-003-17 | 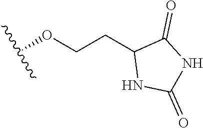 | 674.4 |
| LIT-003-18 | 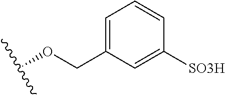 | 718.2 |
| LIT-003-19 | 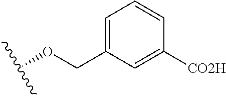 | 682.2 |

The synthesis of tert-butyl (2S,4R)-2-(dimethylcarbamothioyl)-4-hydroxypyrrolidine-1-carboxylate (LIT-003_Int1)

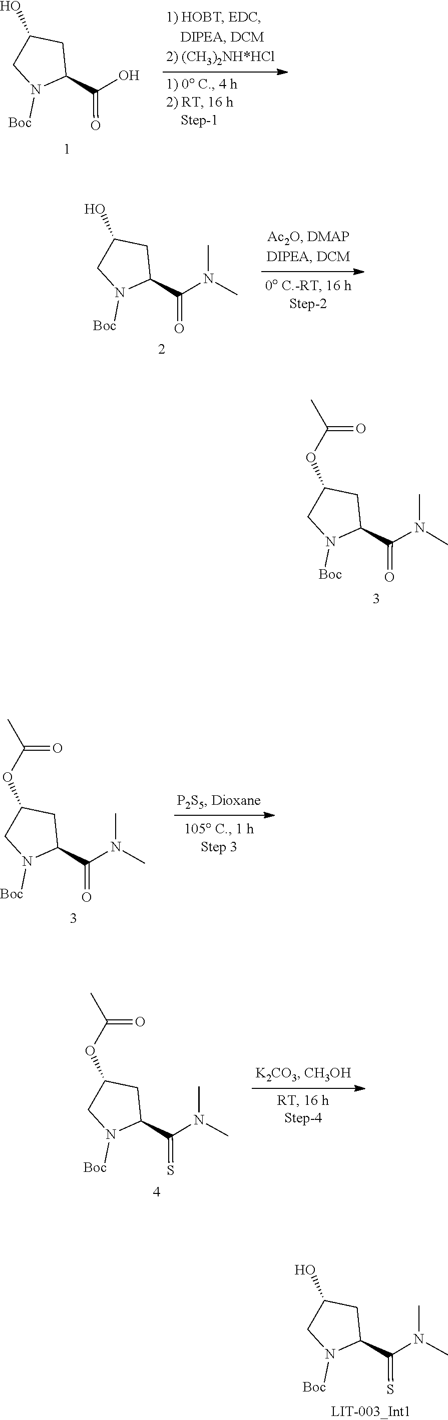 [SMILES: CC(C)(C)OC(=O)N1C[C@H](O)C[C@H]1C(=O)O ; CN(C)C(=O)[C@@H]1C[C@@H](O)CN1C(=O)OC(C)(C)C ; CC(=O)O[C@@H]1C[C@@H](C(=O)N(C)C)N(C(=O)OC(C)(C)C)C1 ; CC(=O)O[C@@H]1C[C@@H](C(=O)N(C)C)N(C(=O)OC(C)(C)C)C1 ; CC(=O)O[C@@H]1C[C@@H](C(=S)N(C)C)N(C(=O)OC(C)(C)C)C1 ; CN(C)C(=S)[C@@H]1C[C@@H](O)CN1C(=O)OC(C)(C)C]

### Step 1. Synthesis of tert-butyl (2S,4R)-2-(dimethylcarbamoyl)-4-hydroxypyrrolidine-1-carboxylate

(2S,4R)-1-[(tert-butoxy)carbonyl]-4-hydroxypyrrolidine-2-carboxylic acid (80 g, 346.15 mmol) was dissolved in DCM and cooled to 0° C., (3-[(ethylimino)methylidene]aminopropyl)dimethylamine hydrochloride (86 g, 449.99 mmol) followed by ethylbis(propan-2-yl)amine (134 g, 1.04 mol, 180.75 ml, 3.0 equiv) and 1H-1,2,3-benzotriazol-1-ol (61 g, 449.99 mmol) were added at 0° C. and the mixture was stirred at rt for 4 h. After 4 h dimethylamine hydrochloride (36 g, 449.99 mmol) was added, and the resulting mixture was stirred at rt for 16 h. After 16 h the mixture was poured into water, The organic layer was washed with NaHSO₄ solution and brine, dried over Na₂SO₄ and evaporated to obtain 60 g with 60% purity. The crude product was purified by FC (system MeOH/MTBE) to afford tert-butyl (2S,4R)-2-(dimethylcarbamoyl)-4-hydroxypyrrolidine-1-carboxylate (41.0 g, 90.0% purity, 142.85 mmol, 41.3% yield).

### Step 2. Synthesis of tert-butyl (2S,4R)-4-(acetyloxy)-2-(dimethylcarbamoyl)pyrrolidine-1-carboxylate

Tert-butyl (2S,4R)-2-(dimethylcarbamoyl)-4-hydroxypyrrolidine-1-carboxylate (10.0 g, 38.74 mmol), ethylbis(propan-2-yl)amine (10.01 g, 77.47 mmol) and N,N-dimethylpyridin-4-amine (473 mg, 3.87 mmol) were dissolved in DCM. Acetyl acetate (4.74 g, 46.48 mmol) was added dropwise at 0° C. and the mixture was stirred at 0° C. for another 10 minutes. After 10 min the mixture was allowed to warm to rt and stirred at rt for 16 h. The mixture was diluted with water, organic layer was washed with 10% K₂CO₃ solution and saturated NaHSO₄ solution, dried over Na₂SO₄ and evaporated to obtain tert-butyl (2S,4R)-4-(acetyloxy)-2-(dimethylcarbamoyl)pyrrolidine-1-carboxylate (8.4 g, 74.0% purity, 20.7 mmol, 53.4% yield).

### Step 3. Synthesis of tert-butyl (2S,4R)-4-(acetyloxy)-2-(dimethylcarbamothioyl)pyrrolidine-1-carboxylate

Tert-butyl (2S,4R)-4-(acetyloxy)-2-(dimethylcarbamoyl)pyrrolidine-1-carboxylate (52.0 g, 173.13 mmol) and [(disulfanylidene-lambda5-phosphanyl)sulfanyl]-lambda5-phosphanedithione (30.72 g, 138.5 mmol) were dissolved in Dioxane and stirred at 105° C. for 1 h. After 1 h the reaction mixture was allowed to cool to rt, concentrated and the residue was purified by FC (system ACN/CHCl₃) to afford tert-butyl (2S,4R)-4-(acetyloxy)-2-(dimethylcarbamothioyl)pyrrolidine-1-carboxylate (10.2 g, 90.0% purity, 29.01 mmol, 16.8% yield).

### Step 4. Synthesis of tert-butyl (2S,4R)-2-(dimethylcarbamothioyl)-4-hydroxypyrrolidine-1-carboxylate

Tert-butyl (2S,4R)-4-(acetyloxy)-2-(dimethylcarbamothioyl)pyrrolidine-1-carboxylate (10.2 g, 32.24 mmol) and dipotassium carbonate (11.11 g, 80.59 mmol) were mixed in MeOH and stirred at rt for 16 h. After 16 h the mixture was concentrated and diluted with CHCl₃. The organic phase was washed with water, dried over Na₂SO₄ and evaporated to obtain tert-butyl (2S,4R)-2-(dimethylcarbamothioyl)-4-hydroxypyrrolidine-1-carboxylate, LIT-003_Int1. Yield: 6.0 g, 66.5%;

### The synthesis of 1-(2-methyl-4-4-methyl-2,4,5,9-tetraazatricyclo[8.4.0.0,3,7]tetradeca-1(14),3(7),5,10,12-pentaene-9-carbonylphenyl)methanamine hydrochloride (LIT-003_Int2)

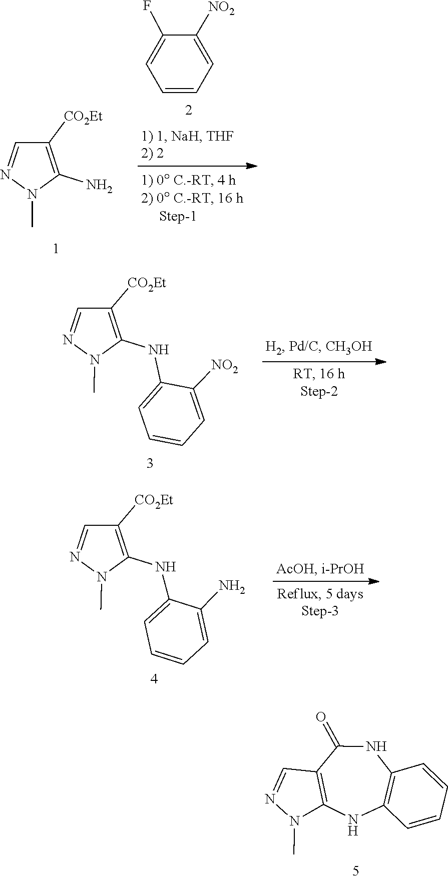 [SMILES: O=[N+]([O-])c1ccccc1F ; CCOC(=O)c1cnn(C)c1N ; CCOC(=O)c1cnn(C)c1Nc1ccccc1[N+](=O)[O-] ; CCOC(=O)c1cnn(C)c1Nc1ccccc1N ; Cn1ncc2c1Nc1ccccc1NC2=O]

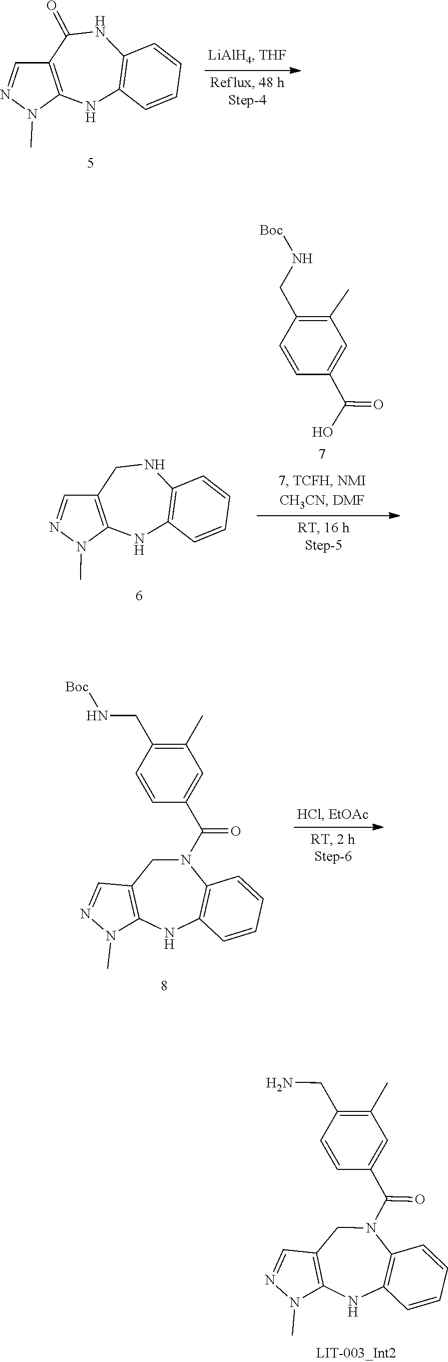 [SMILES: Cn1ncc2c1Nc1ccccc1NC2=O ; Cc1cc(C(=O)O)ccc1CNC(=O)OC(C)(C)C ; Cn1ncc2c1Nc1ccccc1NC2 ; Cc1cc(C(=O)N2Cc3cnn(C)c3Nc3ccccc32)ccc1CNC(=O)OC(C)(C)C ; Cc1cc(C(=O)N2Cc3cnn(C)c3Nc3ccccc32)ccc1CN]

### Step 1. Synthesis of ethyl 1-methyl-5-[(2-nitrophenyl)amino]-1H-pyrazole-4-carboxylate

To a stirred solution of ethyl 5-amino-1-methyl-1H-pyrazole-4-carboxylate (100.0 g, 591.42 mmol) in THF at 0° C. was added sodium hydride (32.64 g, 1.36 mol) portionwise and the reaction mixture was allowed to warm to rt and stirred at rt for 4 h. After 4 h the mixture was cooled to 0° C. and a solution of 1-fluoro-2-nitrobenzene (83.4 g, 591.42 mmol) in THF was added dropwise. The resulting mixture was stirred at rt for 16 h. After 16 h the mixture was poured into water, extracted with EtOAc, organic layer was washed with brine, dried over Na₂SO₄ and evaporated to obtain crude product. The crude product was purified by FC (system ACN/CHCl₃) to afford ethyl 1-methyl-5-[(2-nitrophenyl)amino]-1H-pyrazole-4-carboxylate (115.0 g, 95.0% purity, 376.37 mmol, 63.6% yield).

### Step 2. Synthesis of ethyl 5-[(2-aminophenyl)amino]-1-methyl-1H-pyrazole-4-carboxylate

Ethyl 1-methyl-5-[(2-nitrophenyl)amino]-1H-pyrazole-4-carboxylate (115.0 g, 396.41 mmol) was dissolved in MeOH and 10% palladium (10.5 g, 99.1 mmol) was added at rt. The reaction mixture was stirred in H₂ atmosphere for 16 h. After 16 h the mixture was filtered through celite and the filtrate was evaporated to obtain ethyl 5-[(2-aminophenyl)amino]-1-methyl-1H-pyrazole-4-carboxylate (85.0 g, 96.0% purity, 313.49 mmol, 79.1% yield).

### Step 3. Synthesis of 4-methyl-2,4,5,9-tetraazatricyclo[8.4.0.0,3,7]tetradeca-1(14),3(7),5,10,12-pentaen-8-one

A solution of ethyl 5-[(2-aminophenyl)amino]-1-methyl-1H-pyrazole-4-carboxylate (85.0 g, 326.76 mmol) in acetic acid/2-propanol (1:9, 2000 ml) was heated at reflux for 5 days. After 5 days the reaction mixture was concentrated, and the residue was triturated in ACN. The obtained precipitate was filtered, washed with ACN and dried under reduced pressure to obtain 4-methyl-2,4,5,9-tetraazatricyclo[8.4.0.0,3,7]tetradeca-1(14),3(7),5,10,12-pentaen-8-one (31.0 g, 144.71 mmol, 44.3% yield).

### Step 4. Synthesis of 4-methyl-2,4,5,9-tetraazatricyclo[8.4.0.0,3,7]tetradeca-1(14),3(7),5,10,12-pentaene

To a stirred suspension of lithium(1+) ion alumanuide (19.18 g, 504.48 mmol) in THF under argon atmosphere at rt was added portionwise 4-methyl-2,4,5,9-tetraazatricyclo[8.4.0.0,3,7]tetradeca-1(14),3(7),5,10,12-pentaen-8-one (27.0 g, 126.12 mmol) and the resulting mxiture was heated at reflux for 16 h. After 16 h the mixture was allowed to cool to rt, another portion of lithium(1+) ion alumanuide (19.18 g, 504.48 mmol) was added portionwise at rt and the mixture was again heated at reflux for 32 h. After 32 h the mixture was cooled to 0° C. and 35% ammonia solution (50 ml) was added dropwise. The mixture was stirred at rt for 1 h, filtered through celite and the filtrate was evaporated to obtain the crude product, which was purified by FC (system CHCl₃/MeOH) to afford 4-methyl-2,4,5,9-tetraazatricyclo[8.4.0.0,3,7]tetradeca-1(14),3(7),5,10,12-pentaene (8.1 g, 40.45 mmol, 32.1% yield).

### Step 5. Synthesis of tert-butyl N-[(2-methyl-4-4-methyl-2,4,5,9-tetraazatricyclo[8.4.0.0,3,7]tetradeca-1(14),3(7),5,10,12-pentaene-9-carbonylphenyl)methyl]carbamate

4-Methyl-2,4,5,9-tetraazatricyclo[8.4.0.0,3,7]tetradeca-1(14),3(7),5,10,12-pentaene (1.6 g, 8.0 mmol), 4-([(tert-butoxy)carbonyl]aminomethyl)-3-methylbenzoic acid (2.12 g, 8.0 mmol), 1-methyl-1H-imidazole (1.57 g, 19.19 mmol) and chloro-N,N,N,N-tetramethylformamidinium hexafluorophosphate (2.69 g, 9.59 mmol) were mixed in DMF/AcN (1:1, 30 ml) and the reaction mixture left stirred at rt for 16 h. LCMS from the reaction mixture showed 88% purity. The resulting mixture was purified by FC (gradient CHCl3/AcN) to afford tert-butyl N-[(2-methyl-4-4-methyl-2,4,5,9-tetraazatricyclo[8.4.0.0,3,7]tetradeca-1(14),3(7),5,10,12-pentaene-9-carbonylphenyl)methyl]carbamate (2.6 g, 95.0% purity, 5.52 mmol, 69% yield).

### Step 6. Synthesis of 1-(2-methyl-4-4-methyl-2,4,5,9-tetraazatricyclo[8.4.0.0,3,7]tetradeca-1(14),3(7),5,10,12-pentaene-9-carbonylphenyl)methanamine hydrochloride

Tert-butyl N-[(2-methyl-4-4-methyl-2,4,5,9-tetraazatricyclo[8.4.0.0,3,7]tetradeca-1(14),3(7),5,10,12-pentaene-9-carbonylphenyl)methyl]carbamate (2.6 g, 5.81 mmol) was dissolved in EA (50 ml) and hydrogen chloride (2.09 g, 58.15 mmol, 14.54 ml, 10.0 equiv) was added, reaction mixture was stirred at rt for 2 h and solvent was evaporated. Then to the resulting material was added AcN (50 ml) and the precipitate was filtered to give 1-(2-methyl-4-4-methyl-2,4,5,9-tetraazatricyclo[8.4.0.0,3,7]tetradeca-1(14),3(7),5,10,12-pentaene-9-carbonylphenyl)methanamine hydrochloride (2.1 g, 95.0% purity, 5.2 mmol, 98.8% yield), LIT-003_Int2. Yield: 18.0 mg, 25.7%; Appearance: White solid; HPLC purity: 100%; LCMS Calculated for C₃₁H₃₇N₁₁O₃S: 644.31; Observed: 644.4[M+H]⁺.

### The synthesis of (2S,4R)-2-(dimethylcarbamothioyl)-4-(3-hydroxypropoxy)-N-[(2-methyl-4-4-methyl-2,4,5,9-tetraazatricyclo[8.4.0.0,3,7]tetradeca-1(14),3(7),5,10,12-pentaene-9-carbonylphenyl)methyl]pyrrolidine-1-carboxamide (LIT-003-01)

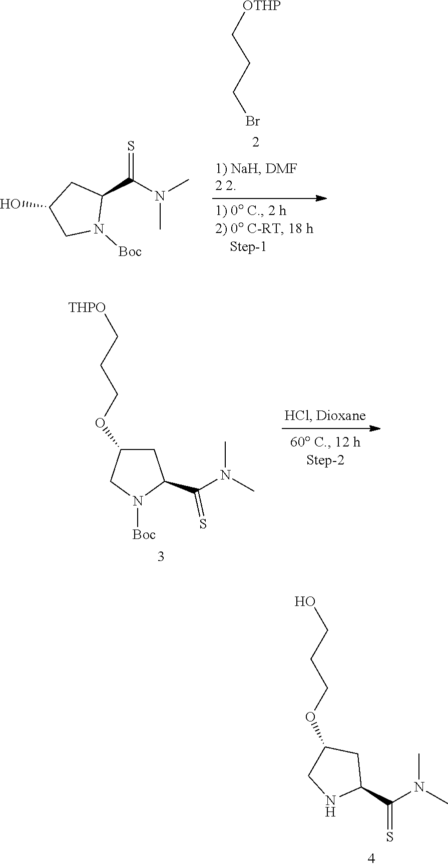

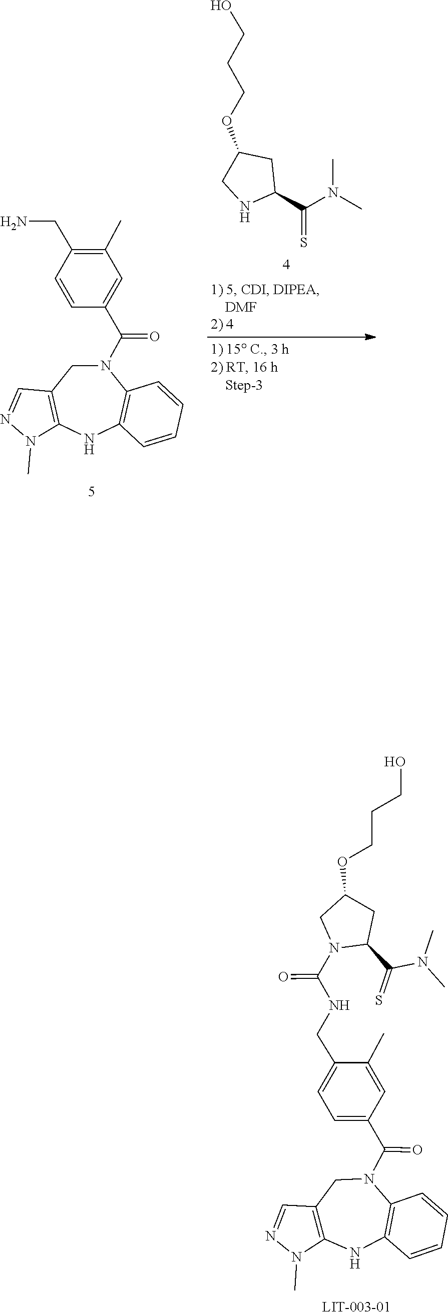 [SMILES: Cc1cc(C(=O)N2Cc3cnn(C)c3Nc3ccccc32)ccc1CN ; CN(C)C(=S)[C@@H]1C[C@@H](OCCCO)CN1 ; Cc1cc(C(=O)N2Cc3cnn(C)c3Nc3ccccc32)ccc1CNC(=O)N1C[C@H](OCCCO)C[C@H]1C(=S)N(C)C]

### Step 1. Synthesis of tert-butyl (2S,4R)-2-(dimethylcarbamothioyl)-4-[3-(oxan-2-yloxy)propoxy]pyrrolidine-1-carboxylate

To a stirred suspension of tert-butyl (2S,4R)-2-(dimethylcarbamothioyl)-4-hydroxypyrrolidine-1-carboxylate (1) (900 mg, 3.28 mmol) in DMF (10 ml) at 0° C. was added sodium hydride (197 mg, 8.2 mmol) and the mixture was stirred at 0° C. for 2 h, then 2-(3-bromopropoxy)oxane (2) (728 mg, 3.28 mmol) was added and the reaction mixture was stirred at 0° C. for another 2 h, allowed to warm to RT and stirred for 16 h. LCMS from the reaction mixture showed 59% of the desired product. The reaction mixture was concentrated and purified by FC (System ACN/CHCl3) to afford tert-butyl (2S,4R)-2-(dimethylcarbamothioyl)-4-[3-(oxan-2-yloxy)propoxy]pyrrolidine-1-carboxylate (3) (300 mg, 93.0% purity, 669.75 μmol, 20.4% yield).

### Step 2. Synthesis of (2S,4R)-4-(3-hydroxypropoxy)-N,N-dimethylpyrrolidine-2-carbothioamide hydrochloride

Tert-butyl (2S,4R)-2-(dimethylcarbamothioyl)-4-[3-(oxan-2-yloxy)propoxy]pyrrolidine-1-carboxylate (3) (300 mg, 720.16 μmol) was dissolved in 4M Dioxane-HCl solution (15 ml) and stirred at 60° C. for 16 h. LCMS from the reaction mixture showed 90% of the desired product. The reaction mixture was concentrated to obtain (2S,4R)-4-(3-hydroxypropoxy)-N, N-dimethylpyrrolidine-2-carbothioamide hydrochloride (4) (160 mg, 90.0% purity, 535.71 μmol, 86.2% yield).

### Step 3. Synthesis of (2S,4R)-2-(dimethylcarbamothioyl)-4-(3-hydroxypropoxy)-N-[(2-methyl-4-4-methyl-2,4,5,9-tetraazatricyclo[8.4.0.0,3,7]tetradeca-1(14),3(7),5,10,12-pentaene-9-carbonylphenyl)methyl]pyrrolidine-1-carboxamide

1-(2-Methyl-4-4-methyl-2,4,5,9-tetraazatricyclo[8.4.0.0,3,7]tetradeca-1(14),3(7),5,10,12-pentaene-9-carbonylphenyl)methanamine (5) (148 mg, 425.92 μmol), 1-(1H-imidazole-1-carbonyl)-1H-imidazole (138 mg, 851.83 μmol) and ethylbis(propan-2-yl)amine (550 mg, 4.26 mmol, 740.0 μl, 10.0 equiv) were dissolved in DMF (5 ml) and stirred at 15° C. for 3 h. (2S,4R)-4-(3-hydroxypropoxy)-N, N-dimethylpyrrolidine-2-carbothioamide hydrochloride (4) (160 mg, 595.23 μmol) was added to the mixture and it was stirred at RT for 16 h. LCMS from the reaction mixture showed 71% of the desired product. The reaction mixture was purified by HPLC (SYSTEM 0-2-8 min 8-15-30% H₂O/ACN, 30 ml\min (loading pump 4 ml ACN), target mass 605 column: CHROMATOREX C18 100*19 mm, 5 microM) to afford (2S,4R)-2-(dimethylcarbamothioyl)-4-(3-hydroxypropoxy)-N-[(2-methyl-4-4-methyl-2,4,5,9-tetraazatricyclo[8.4.0.0,3,7]tetradeca-1(14),3(7),5,10,12-pentaene-9-carbonylphenyl)methyl]pyrrolidine-1-carboxamide, LIT-003-01. Yield: 162 mg, 62.8%; Appearance: Yellow solid; HPLC purity: 100%; LCMS Calculated for C₃₁H₃₉N₇O₄S: 606.32; Observed: 606.2[M+H]⁺.

### The synthesis of (2S,4R)-2-(dimethylcarbamothioyl)-4-(4-hydroxybutoxy)-N-[(2-methyl-4-4-methyl-2,4,5,9-tetraazatricyclo[8.4.0.0,3,7]tetradeca-1(14),3(7),5,10,12-pentaene-9-carbonylphenyl)methyl]pyrrolidine-1-carboxamide (LIT-003-02)

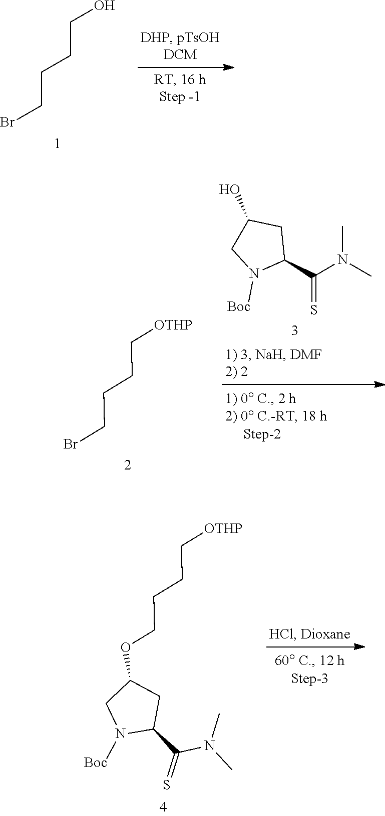

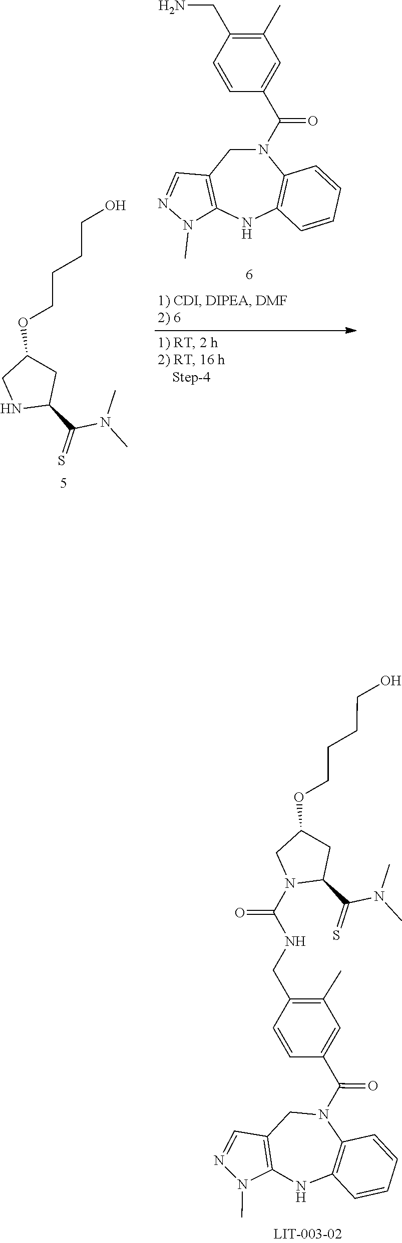 [SMILES: CN(C)C(=S)[C@@H]1C[C@@H](OCCCCO)CN1 ; Cc1cc(C(=O)N2Cc3cnn(C)c3Nc3ccccc32)ccc1CN ; Cc1cc(C(=O)N2Cc3cnn(C)c3Nc3ccccc32)ccc1CNC(=O)N1C[C@H](OCCCCO)C[C@H]1C(=S)N(C)C]

### Step 1. Synthesis of 2-(4-bromobutoxy)oxane

3,4-Dihydro-2H-pyran (1) (3.96 g, 47.06 mmol, 4.29 ml, 1.3 equiv) was added dropwise to a solution of 4-bromobutan-1-ol (5.5 g, 36.19 mmol) and 4-methylbenzene-1-sulfonic acid hydrate (69 mg, 361.95 μmol) in DCM (50 ml) and the mixture was stirred at RT for 16 h.

After removing the solvent, the residue was purified by FC (System MTBE-Hexane) to afford 2-(4-bromobutoxy)oxane (2.9 g, 95.0% purity, 11.62 mmol, 32.1% yield).

### Step 2. Synthesis of tert-butyl (2S,4R)-2-(dimethylcarbamothioyl)-4-[4-(oxan-2-yloxy)butoxy]pyrrolidine-1-carboxylate

To a stirred suspension of tert-butyl (2S,4R)-2-(dimethylcarbamothioyl)-4-hydroxypyrrolidine-1-carboxylate (1.0 g, 3.64 mmol) in DMF (10 ml) at 0° C. was added sodium hydride (219 mg, 9.11 mmol) and the mixture was stirred at 0° C. for 2 h, then 2-(4-bromobutoxy)oxane (860 mg, 3.64 mmol) was added and the reaction mixture was stirred at 0° C. for another 2 h, allowed to warm to RT and stirred for 16 h. LCMS from the reaction mixture showed 59% of the desired product. The reaction mixture was concentrated and purified by FC (System ACN/CHCl3) to afford tert-butyl (2S,4R)-2-(dimethylcarbamothioyl)-4-[4-(oxan-2-yloxy)butoxy]pyrrolidine-1-carboxylate (580 mg, 98.0% purity, 1.32 mmol, 36.2% yield)

### Step 3. Synthesis of (2S,4R)-4-(4-hydroxybutoxy)-N,N-dimethylpyrrolidine-2-carbothioamide hydrochloride

Tert-butyl (2S,4R)-2-(dimethylcarbamothioyl)-4-[4-(oxan-2-yloxy)butoxy]pyrrolidine-1-carboxylate (580 mg, 1.35 mmol) was dissolved in 4M Dioxane-HCl solution and stirred at 60° C. for 16 h. LCMS from the reaction mixture showed 65% of the desired product. The reaction mixture was concentrated to obtain (2S,4R)-4-(4-hydroxybutoxy)-N,N-dimethylpyrrolidine-2-carbothioamide hydrochloride (380 mg, 65.0% purity, 873.31 μmol, 64.8% yield).

### Step 4. Synthesis of (2S,4R)-2-(dimethylcarbamothioyl)-4-(4-hydroxybutoxy)-N-[(2-methyl-4-4-methyl-2,4,5,9-tetraazatricyclo[8.4.0.0,3,7]tetradeca-1(14),3(7),5,10,12-pentaene-9-carbonylphenyl)methyl]pyrrolidine-1-carboxamide

1-(2-Methyl-4-4-methyl-2,4,5,9-tetraazatricyclo[8.4.0.0,3,7]tetradeca-1(14),3(7),5,10,12-pentaene-9-carbonylphenyl)methanamine (114 mg, 327.54 μmol), 1-(1H-imidazole-1-carbonyl)-1H-imidazole (106 mg, 655.07 μmol) and ethylbis(propan-2-yl)amine (423 mg, 3.27 mmol, 570.0 μl, 10.0 equiv) were dissolved in DMF (5 ml) and stirred at 15° C. for 3 h. (2S,4R)-4-(4-hydroxybutoxy)-N,N-dimethylpyrrolidine-2-carbothioamide hydrochloride (130 mg, 458.55 μmol) was added to the mixture and it was stirred at rt for 16 h. LCMS from the reaction mixture showed 71% of the desired product. The reaction mixture was purified by HPLC (SYSTEM 0-2-8 min 27-35-100% H₂O/ACN 30 ml/min (loading pump 4 ml ACN), target mass 619 column: CHROMATOREX C18 SMB100-5T 100*19 mm, 5 microM) to afford 85% purity only. The material was repurified by SFC (Viridis 2-EP (150×4.6 mm, 5mkm)—01233120816103—13 Mobile Phase: Hexane-[IPA:MeOH], [50:50] 10-60 ! 32 Flow Rate: 1.0 ml/min) to afford (2S,4R)-2-(dimethylcarbamothioyl)-4-(4-hydroxybutoxy)-N-[(2-methyl-4-4-methyl-2,4,5,9-tetraazatricyclo[8.4.0.0,3,7]tetradeca-1(14),3(7),5,10,12-pentaene-9-carbonylphenyl)methyl]pyrrolidine-1-carboxamide, LIT-003-02. Yield: 10.0 mg, 4.9%; Appearance: Beige solid; HPLC purity: 100%; LCMS Calculated for C₃₂H₄₁N₇O₄S: 620.34; Observed: 620.2[M+H]⁺.

### The synthesis of (2S,4R)-2-(dimethylcarbamothioyl)-4-[2-(2-hydroxyethoxy)ethoxy]-N-[(2-methyl-4-4-methyl-2,4,5,9-tetraazatricyclo[8.4.0.0,3,7]tetradeca-1(14),3(7),5,10,12-pentaene-9-carbonylphenyl)methyl]pyrrolidine-1-carboxamide (LIT-003-03)

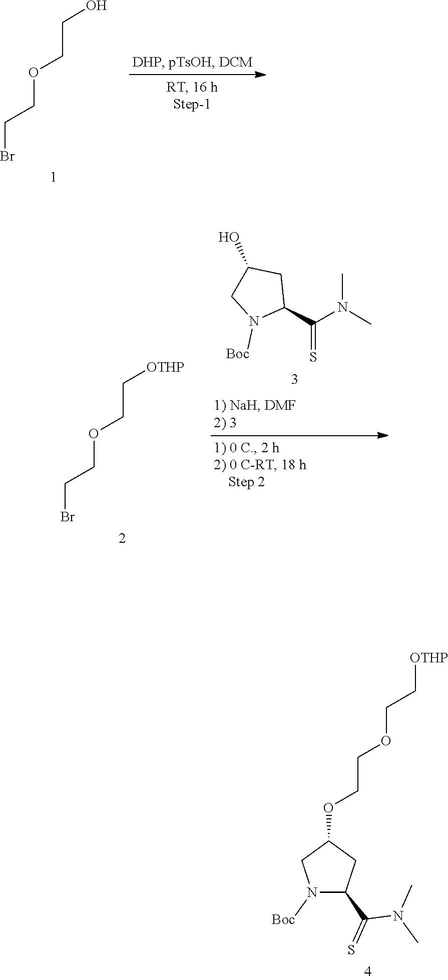

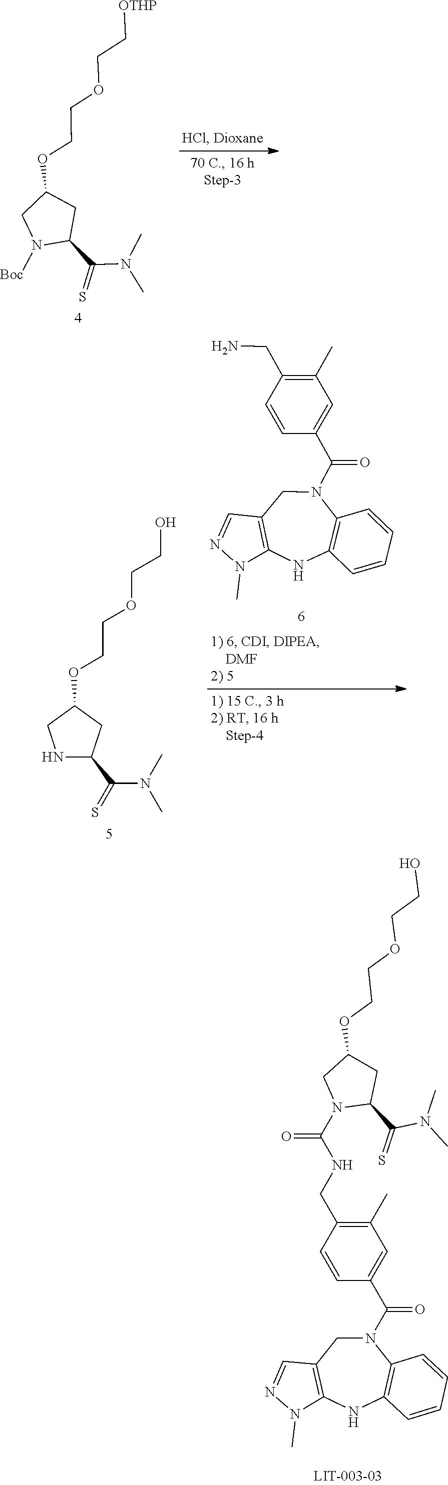

### Step 1. Synthesis of 2-[2-(2-bromoethoxy)ethoxy]oxane

3,4-Dihydro-2H-pyran (1) (3.9 g, 46.43 mmol, 4.23 ml, 1.5 equiv) was added dropwise to a solution of 2-(2-bromoethoxy)ethan-1-ol (5.2 g, 30.96 mmol) and 4-methylbenzene-1-sulfonic acid hydrate (59 mg, 309.56 μmol) in DCM (100 ml) and the mixture was stirred at RT for 16 h. After removing the solvent, the residue was purified by FC (System MTBE-Hexane) to afford 2-[2-(2-bromoethoxy)ethoxy]oxane (2) (4.0 g, 95.0% purity, 15.01 mmol, 48.5% yield)

### Step 2. Synthesis of tert-butyl (2S,4R)-2-(dimethylcarbamothioyl)-4-2-[2-(oxan-2-yloxy)ethoxy]ethoxypyrrolidine-1-carboxylate

To a stirred suspension of tert-butyl (2S,4R)-2-(dimethylcarbamothioyl)-4-hydroxypyrrolidine-1-carboxylate (3) (800 mg, 2.92 mmol) in DMF (10 ml) at 0° C. was added sodium hydride (175 mg, 7.29 mmol) and the mixture was stirred at 0° C. for 2 h, then 2-[2-(2-bromoethoxy)ethoxy]oxane (2) (735 mg, 2.92 mmol) was added and the reaction mixture was stirred at 0° C. for another 2 h, allowed to warm to rt and stirred for 16 h. LCMS from the reaction mixture showed 40% of the desired product. The reaction mixture was concentrated and purified by FC (System ACN/CHCl₃) to afford tert-butyl (2S,4R)-2-(dimethylcarbamothioyl)-4-2-[2-(oxan-2-yloxy)ethoxy]ethoxypyrrolidine-1-carboxylate (4) (200 mg, 91.0% purity, 407.52 μmol, 14% yield)

### Step 3. Synthesis of (2S,4R)-4-[2-(2-hydroxyethoxy)ethoxy]-N,N-dimethylpyrrolidine-2-carbothioamide hydrochloride

Tert-butyl (2S,4R)-2-(dimethylcarbamothioyl)-4-2-[2-(oxan-2-yloxy)ethoxy]ethoxypyrrolidine-1-carboxylate (4) (200 mg, 447.83 μmol) was dissolved in 4M Dioxane-HCl solution and stirred at 70° C. for 16 h. LCMS from the reaction mixture showed 78% of the desired product. The reaction mixture was concentrated to obtain (2S,4R)-4-[2-(2-hydroxyethoxy)ethoxy]-N,N-dimethylpyrrolidine-2-carbothioamide hydrochloride (5) (130 mg, 78.0% purity, 339.32 μmol, 75.7% yield).

### Step 4. Synthesis of (2S,4R)-2-(dimethylcarbamothioyl)-4-[2-(2-hydroxyethoxy)ethoxy]-N-[(2-methyl-4-4-methyl-2,4,5,9-tetraazatricyclo[8.4.0.0,3,7]tetradeca-1(14),3(7),5,10,12-pentaene-9-carbonylphenyl)methyl]pyrrolidine-1-carboxamide

1-(2-Methyl-4-4-methyl-2,4,5,9-tetraazatricyclo[8.4.0.0,3,7]tetradeca-1(14),3(7),5,10,12-pentaene-9-carbonylphenyl)methanamine (6) (108 mg, 311.43 μmol), 1-(1H-imidazole-1-carbonyl)-1H-imidazole (101 mg, 622.86 μmol) and ethylbis(propan-2-yl)amine (402 mg, 3.11 mmol) were dissolved in DMF (5 ml) and stirred at 15° C. for 3 h. (2S,4R)-4-[2-(2-hydroxyethoxy)ethoxy]-N,N-dimethylpyrrolidine-2-carbothioamide hydrochloride (5) (130 mg, 435.03 μmol) was added to the mixture and it was stirred at RT for 16 h. LCMS from the reaction mixture showed 70% of the desired product. The reaction mixture was purified by HPLC (SYSTEM 0-2-8 min 7-15-30% H2O/ACN 30 ml/min (loading pump 4 ml ACN), target mass 635 column: CHROMATOREX C18 SMB100-5T 100*19 mm, 5 microM) to afford (2S,4R)-2-(dimethylcarbamothioyl)-4-[2-(2-hydroxyethoxy)ethoxy]-N-[(2-methyl-4-4-methyl-2,4,5,9-tetraazatricyclo[8.4.0.0,3,7]tetradeca-1(14),3(7),5,10,12-pentaene-9-carbonylphenyl)methyl]pyrrolidine-1-carboxamide, LIT-003-03. Yield: 17.0 mg, 8.5%; Appearance: Colorless solid; HPLC purity: 98.60%; LCMS Calculated for C₃₂H₄₁N₇O₅S: 636.33; Observed: 636.2[M+H]⁺.

### The synthesis of 5-chloro-N-(3-chloro-5-cyclopropylphenyl)-2-methoxy-4-methylbenzene-1-sulfonamide (LIT-003-04)

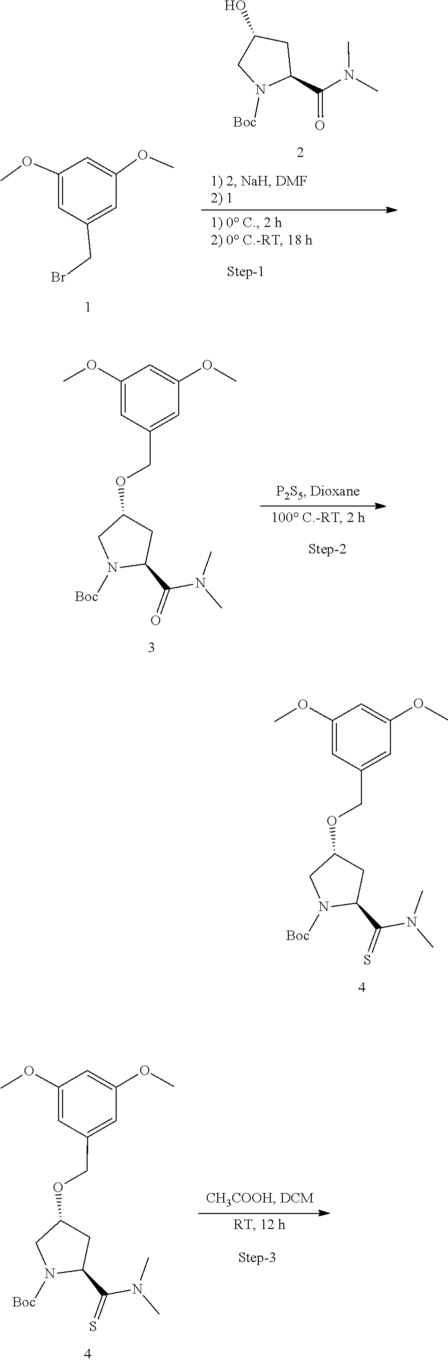

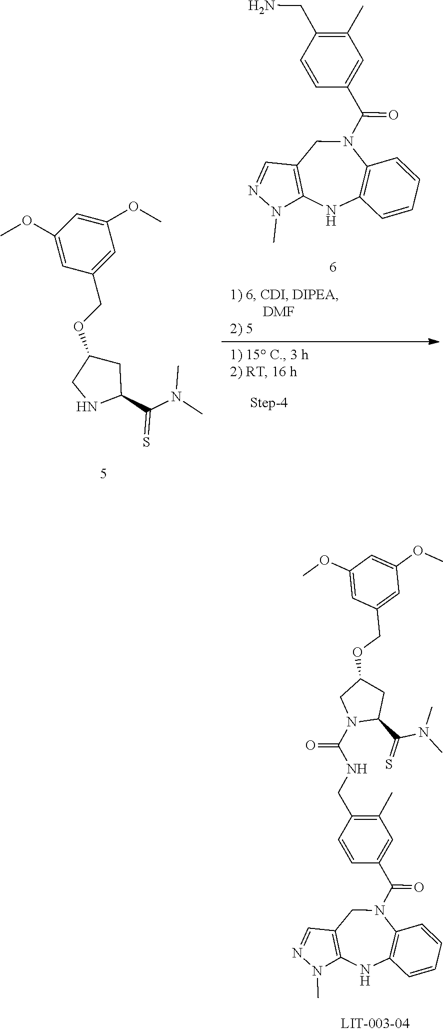

### Step 1. Synthesis of tert-butyl (2S,4R)-4-[(3,5-dimethoxyphenyl)methoxy]-2-(dimethylcarbamoyl)pyrrolidine-1-carboxylate

To a stirred solution of tert-butyl (2S,4R)-2-(dimethylcarbamoyl)-4-hydroxypyrrolidine-1-carboxylate (2) (3.0 g, 11.62 mmol) in DMF at 0° C. was added sodium hydride (1.12 g, 46.48 mmol) and the mixture was stirred at 0° C. for 2 h, then 1-(bromomethyl)-3,5-dimethoxybenzene (1) (2.67 g, 11.62 mmol) was added and the reaction mixture was stirred at 0° C. for another 2 h, allowed to warm to rt and stirred for 16 h. LCMS from the reaction mixture showed 47% of the desired product. The reaction mixture was concentrated and purified by FC (ACN/CHCl3) to afford tert-butyl (2S,4R)-4-[(3,5-dimethoxyphenyl)methoxy]-2-(dimethylcarbamoyl)pyrrolidine-1-carboxylate (3) (500 mg, 70.0% purity, 856.82 μmol, 7.4% yield).

### Step 2. Synthesis of tert-butyl (2S,4R)-4-[(3,5-dimethoxyphenyl)methoxy]-2-(dimethylcarbamothioyl)pyrrolidine-1-carboxylate

Tert-butyl (2S,4R)-4-[(3,5-dimethoxyphenyl)methoxy]-2-(dimethylcarbamoyl)pyrrolidine-1-carboxylate (3) (500 mg, 1.22 mmol) and [(disulfanylidene-lambda5-phosphanyl)sulfanyl]-lambda5-phosphanedithione (217 mg, 979.85 μmol) were dissolved in Dioxane and stirred at 100° C. for 1 h. After 1 h the reaction mixture was allowed to cool to rt, concentrated and the residue was purified by HPLC (SYSTEM 23-30-90 0-2-9 min H2O/ACN flow 30 ml/min ((loading pump 4 ml ACN) target mass 424 column: XBRIDGE 100*19 mm, 5 microM) to afford tert-butyl (2S,4R)-4-[(3,5-dimethoxyphenyl)methoxy]-2-(dimethylcarbamothioyl)pyrrolidine-1-carboxylate (4) (130 mg, 80.0% purity, 244.96 μmol, 20% yield).

### Step 3. Synthesis of (2S,4R)-4-[(3,5-dimethoxyphenyl)methoxy]-N,N-dimethylpyrrolidine-2-carbothioamide

Tert-butyl (2S,4R)-4-[(3,5-dimethoxyphenyl)methoxy]-2-(dimethylcarbamothioyl)pyrrolidine-1-carboxylate (4) (130 mg, 306.2 μmol) was dissolved in DCM (2 ml) and TFA (1 ml) was added to the mixture at 0° C. The resulting mixture was stirred at rt for 12 h. LCMS from the reaction mixture indicated 75% of the desired product. The mixture was concentrated to obtain (2S,4R)-4-[(3,5-dimethoxyphenyl)methoxy]-N, N-dimethylpyrrolidine-2-carbothioamide (5) (120 mg, 75.0% purity, 277.4 μmol, 90.6% yield) as a TFA salt.

### Step 4. Synthesis of (2S,4R)-4-[(3,5-dimethoxyphenyl)methoxy]-2-(dimethylcarbamothioyl)-N-[(2-methyl-4-4-methyl-2,4,5,9-tetraazatricyclo[8.4.0.0,3,7]tetradeca-1(14),3(7),5,10,12-pentaene-9-carbonylphenyl)methyl]pyrrolidine-1-carboxamide

1-(2-Methyl-4-4-methyl-2,4,5,9-tetraazatricyclo[8.4.0.0,3,7]tetradeca-1(14),3(7),5,10,12-pentaene-9-carbonylphenyl)methanamine (6) (92 mg, 263.67 μmol), 1-(1H-imidazole-1-carbonyl)-1H-imidazole (86 mg, 527.34 μmol) and ethylbis(propan-2-yl)amine (341 mg, 2.64 mmol) were dissolved in DMF (5 ml) and stirred at 15° C. for 3 h. (2S,4R)-4-[(3,5-dimethoxyphenyl)methoxy]-N, N-dimethylpyrrolidine-2-carbothioamide (5) (120 mg, 369.87 μmol) was added to the mixture and it was stirred at rt for 16 h. LCMS from the reaction mixture showed 49% of the desired product. The reaction mixture was purified by HPLC (SYSTEM 0-2-8 min 23-30-50% H2O/ACN, 30 ml\min (loading pump 4 ml ACN), target mass 697 column: Chromatorex C18 SMB100-5T 100*19 mm, 5 microM) to afford (2S,4R)-4-[(3,5-dimethoxyphenyl)methoxy]-2-(dimethylcarbamothioyl)-N-[(2-methyl-4-4-methyl-2,4,5,9-tetraazatricyclo[8.4.0.0,3,7]tetradeca-1(14),3(7),5,10,12-pentaene-9-carbonylphenyl)methyl]pyrrolidine-1-carboxamide, LIT-003-04. Yield: 32.6 mg, 17.7%; Appearance: Beige solid; HPLC purity: 100%; LCMS Calculated for C₃₇H₄₃N₇O₅S: 698.35; Observed: 698.2[M+H]⁺.

### The synthesis of (2S,4R)-2-(dimethylcarbamothioyl)-4-[(3-hydroxyphenyl)methoxy]-N-[(2-methyl-4-4-methyl-2,4,5,9-tetraazatricyclo[8.4.0.0,3,7]tetradeca-1(14),3(7),5,10,12-pentaene-9-carbonylphenyl)methyl]pyrrolidine-1-carboxamide (LIT-003-05)

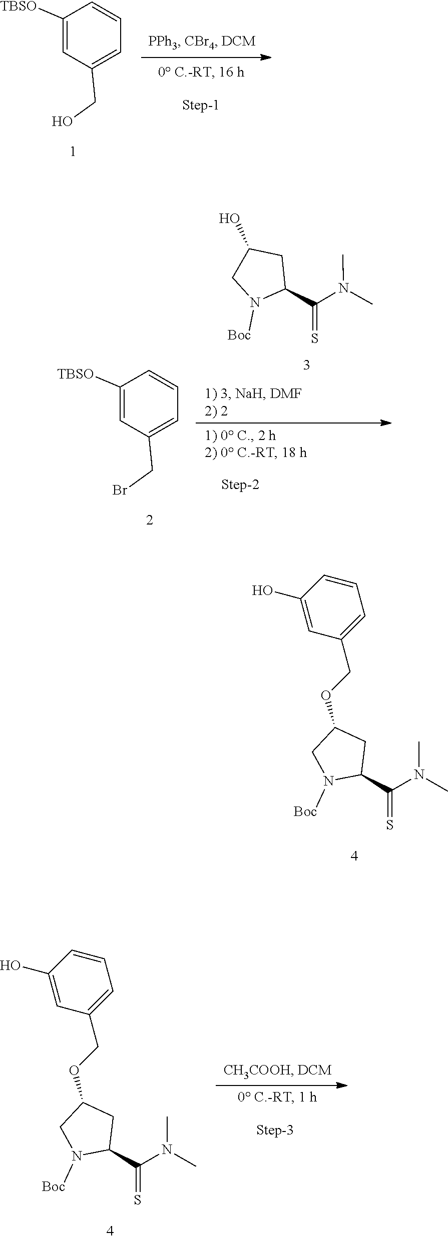 [SMILES: CC(C)(C)[Si](C)(C)Oc1cccc(CO)c1 ; CN(C)C(=S)[C@@H]1C[C@@H](O)CN1C(=O)OC(C)(C)C ; CC(C)(C)[Si](C)(C)Oc1cccc(CBr)c1 ; CN(C)C(=S)[C@@H]1C[C@@H](OCc2cccc(O)c2)CN1C(=O)OC(C)(C)C ; CN(C)C(=S)[C@@H]1C[C@@H](OCc2cccc(O)c2)CN1C(=O)OC(C)(C)C]

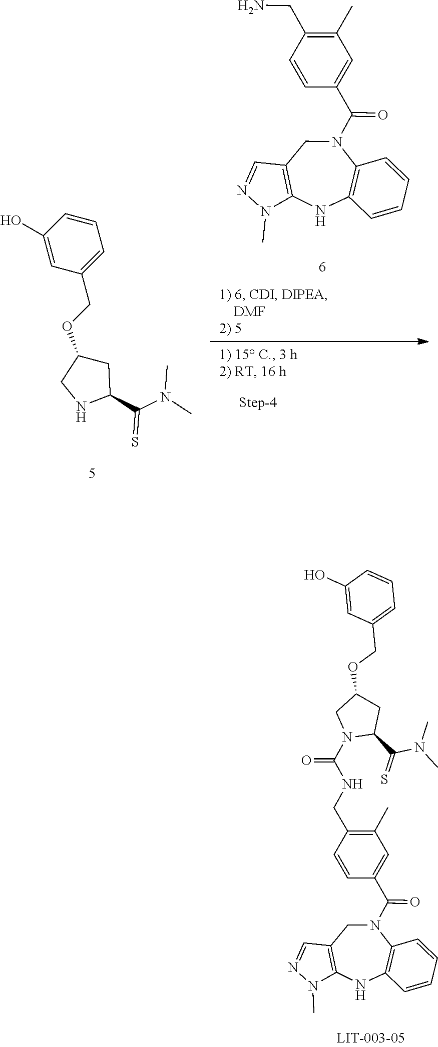 [SMILES: CN(C)C(=S)[C@@H]1C[C@@H](OCc2cccc(O)c2)CN1 ; Cc1cc(C(=O)N2Cc3cnn(C)c3Nc3ccccc32)ccc1CN ; Cc1cc(C(=O)N2Cc3cnn(C)c3Nc3ccccc32)ccc1CNC(=O)N1C[C@H](OCc2cccc(O)c2)C[C@H]1C(=S)N(C)C]

### Step 1. Synthesis of afford [3-(bromomethyl)phenoxy](tert-butyl)dimethylsilane

To a stirred solution of 3-[(tert-butyldimethylsilyl)oxy]phenylmethanol (1) (2.0 g, 8.4 mmol) in DCM at 0° C. was added triphenylphosphane (2.86 g, 10.92 mmol) and tetrabromomethane (3.58 g, 10.92 mmol) portion-wise. The resulting mixture was stirred at rt for 16 h. The solvent was evaporated and the residue was purified by FC (System MTBE/Hexane) to afford [3-(bromomethyl)phenoxy](tert-butyl)dimethylsilane (2) (1.3 g, 95.0% purity, 4.1 mmol, 48.8% yield)

### Step 2. Synthesis of tert-butyl (2S,4R)-2-(dimethylcarbamothioyl)-4-[(3-hydroxyphenyl)methoxy]pyrrolidine-1-carboxylate

To a stirred suspension of tert-butyl (2S,4R)-2-(dimethylcarbamothioyl)-4-hydroxypyrrolidine-1-carboxylate (3) (400 mg, 1.46 mmol) in DMF at 0° C. was added sodium hydride (140 mg, 5.83 mmol) and the mixture was stirred at 0° C. for 2 h, then [3-(bromomethyl)phenoxy](tert-butyl)dimethylsilane (2) (438 mg, 1.46 mmol) was added and the reaction mixture was stirred at 0° C. for another 2 h, allowed to warm to RT and stirred for 16 h. LCMS from the reaction mixture showed 21% of the desired product. The reaction mixture was concentrated and purified by HPLC (SYSTEM 0-2-8 min 27-35-100% H2O/ACN 30 ml/min (loading pump 4 ml ACN), target mass 380 column: CHROMATOREX C18 SMB100-5T 100*19 mm, 5 microM) to afford tert-butyl (2S,4R)-2-(dimethylcarbamothioyl)-4-[(3-hydroxyphenyl)methoxy]pyrrolidine-1-carboxylate (4) (36 mg, 84.0% purity, 79.47 μmol, 5.4% yield).

### Step 3. Synthesis of (2S,4R)-4-[(3-hydroxyphenyl)methoxy]-N,N-dimethylpyrrolidine-2-carbothioamide

Tert-butyl (2S,4R)-2-(dimethylcarbamothioyl)-4-[(3-hydroxyphenyl)methoxy]pyrrolidine-1-carboxylate (4) (36 mg, 94.61 μmol) was dissolved in DCM (2 ml) and TFA (1 ml) was added to the mixture at 0° C. LCMS from the reaction mixture indicated 76% of the desired product. The resulting mixture was stirred at rt for 1 h. The mixture was concentrated to obtain (2S,4R)-4-[(3-hydroxyphenyl)methoxy]-N,N-dimethylpyrrolidine-2-carbothioamide (5) (30 mg, 76.0% purity, 81.32 μmol, 86% yield) as a TFA salt.

### Step 4. Synthesis of (2S,4R)-2-(dimethylcarbamothioyl)-4-[(3-hydroxyphenyl)methoxy]-N-[(2-methyl-4-4-methyl-2,4,5,9-tetraazatricyclo[8.4.0.0,3,7]tetradeca-1(14),3(7),5,10,12-pentaene-9-carbonylphenyl)methyl]pyrrolidine-1-carboxamide

1-(2-Methyl-4-4-methyl-2,4,5,9-tetraazatricyclo[8.4.0.0,3,7]tetradeca-1(14),3(7),5,10,12-pentaene-9-carbonylphenyl)methanamine (6) (26 mg, 76.48 μmol), 1-(1H-imidazole-1-carbonyl)-1H-imidazole (24.79 mg, 153 μmol) and ethylbis(propan-2-yl)amine (99 mg, 764.77 μmol) were dissolved in DMF (5 ml) and stirred at 15° C. for 3 h. (2S,4R)-4-[(3-hydroxyphenyl)methoxy]-N, N-dimethylpyrrolidine-2-carbothioamide (5) (30 mg, 107.0 μmol) was added to the mixture and it was stirred at rt for 16 h. LCMS from the reaction mixture showed 86% of the desired product. The reaction mixture was purified by HPLC (SYSTEM 0-2-8 min 18-25-50% H₂O/ACN 30 ml/min (loading pump 4 ml ACN), target mass 653 column: CHROMATOREX C18 SMB100-5T 100*19 mm, 5 microM) to afford (2S,4R)-2-(dimethylcarbamothioyl)-4-[(3-hydroxyphenyl)methoxy]-N-[(2-methyl-4-4-methyl-2,4,5,9-tetraazatricyclo[8.4.0.0,3,7]tetradeca-1(14),3(7),5,10,12-pentaene-9-carbonylphenyl)methyl]pyrrolidine-1-carboxamide, LIT-003-05 Yield: 30.0 mg, 57.6%; Appearance: Brown solid; HPLC purity: 97.94%; LCMS Calculated for C₃₅H₃₉N₇O₄S: 653.28; Observed: 653[M+H]⁺.

### The synthesis of (2S,4R)-2-(dimethylcarbamothioyl)-4-[(4-hydroxyphenyl)methoxy]-N-[(2-methyl-4-4-methyl-2,4,5,9-tetraazatricyclo[8.4.0.0,3,7]tetradeca-1(14),3(7),5,10,12-pentaene-9-carbonylphenyl)methyl]pyrrolidine-1-carboxamide (LIT-003-07)

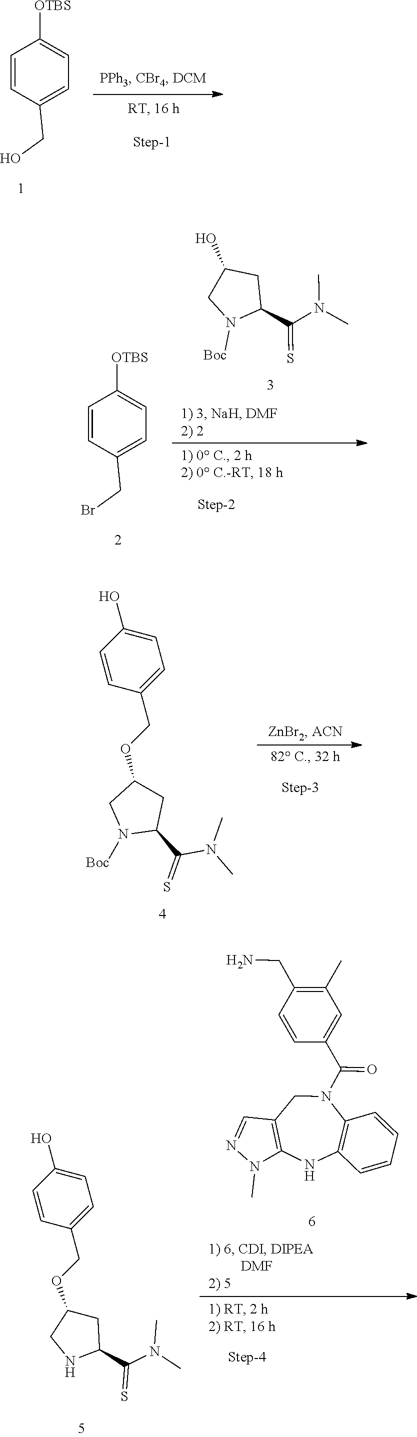 [SMILES: CC(C)(C)[Si](C)(C)Oc1ccc(CO)cc1 ; CC(C)(C)[Si](C)(C)Oc1ccc(CBr)cc1 ; CN(C)C(=S)[C@@H]1C[C@@H](O)CN1C(=O)OC(C)(C)C ; CN(C)C(=S)[C@@H]1C[C@@H](OCc2ccc(O)cc2)CN1C(=O)OC(C)(C)C ; CN(C)C(=S)[C@@H]1C[C@@H](OCc2ccc(O)cc2)CN1 ; Cc1cc(C(=O)N2Cc3cnn(C)c3Nc3ccccc32)ccc1CN]

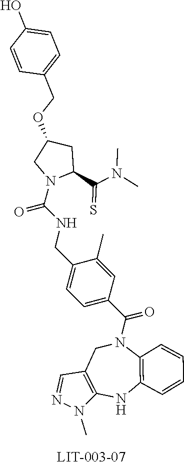 [SMILES: Cc1cc(C(=O)N2Cc3cnn(C)c3Nc3ccccc32)ccc1CNC(=O)N1C[C@H](OCc2ccc(O)cc2)C[C@H]1C(=S)N(C)C]

### Step 1. Synthesis of [4-(bromomethyl)phenoxy](tert-butyl)dimethyl silane

To a stirred solution of 4-[(tert-butyldimethylsilyl)oxy]phenylmethanol (1) (2.0 g, 8.4 mmol) in DCM at 0° C. was added triphenylphosphane (2.86 g, 10.92 mmol, 1, 1.3 equiv) and tetrabromomethane (3.58 g, 10.92 mmol) portion wise. The resulting mixture was stirred at rt for 16 h. The solvent was evaporated and the residue was purified by FC (System MTBE/Hexane) to afford [4-(bromomethyl)phenoxy](tert-butyl)dimethylsilane (2) (600 mg, 95.0% purity, 1.89 mmol, 22.5% yield).

### Step 2. Synthesis of tert-butyl (2S,4R)-2-(dimethylcarbamothioyl)-4-[(4-hydroxyphenyl)methoxy]pyrrolidine-1-carboxylate

To a stirred suspension of tert-butyl (2S,4R)-2-(dimethylcarbamothioyl)-4-hydroxypyrrolidine-1-carboxylate (3) (640 mg, 2.33 mmol) in DMF (10 ml) at 0° C. was added sodium hydride (224 mg, 9.34 mmol) and the mixture was stirred at 0° C. for 2 h, then [4-(bromomethyl)phenoxy](tert-butyl)dimethylsilane (2) (700 mg, 2.33 mmol) was added and the reaction mixture was stirred at 0° C. for another 2 h, allowed to warm to rt and stirred for 16 h. LCMS from the reaction mixture showed 56% of the desired product. The reaction mixture was purified by HPLC (SYSTEM 0-2-8 min 13-20-40% H₂O/ACN 30 ml\min (loading pump 4 ml ACN), target mass 380 column: CHROMATOREX C18 100*19 mm, 5 microM) to afford tert-butyl (2S,4R)-2-(dimethylcarbamothioyl)-4-[(4-hydroxyphenyl)methoxy]pyrrolidine-1-carboxylate (4) (130.0 mg, 97.0% purity, 331.4 μmol, 14.2% yield).

### Step 3. Synthesis of (2S,4R)-4-[(4-hydroxyphenyl)methoxy]-N,N-dimethylpyrrolidine-2-carbothioamide

Tert-butyl (2S,4R)-2-(dimethylcarbamothioyl)-4-[(4-hydroxyphenyl)methoxy]pyrrolidine-1-carboxylate (4) (72 mg, 189.22 μmol) and dibromozinc (420 mg, 1.89 mmol) were dissolved in ACN and stirred at 82° C. for 32 h. LCMS from the reaction mixture indicated 43% of the desired product. The mixture was concentrated and the residue was purified by HPLC (SYSTEM 0-2-8 min 13-20-40% H₂O/ACN 30 ml\min (loading pump 4 ml ACN), target mass 380 column: CHROMATOREX C18 100*19 mm, 5 microM) to afford (2S,4R)-4-[(4-hydroxyphenyl)methoxy]-N,N-dimethylpyrrolidine-2-carbothioamide (5) (8.0 mg, 94.0% purity, 26.82 μmol, 14.2% yield).

### Step 3. Synthesis of (2S,4R)-2-(dimethylcarbamothioyl)-4-[(4-hydroxyphenyl)methoxy]-N-[(2-methyl-4-4-methyl-2,4,5,9-tetraazatricyclo[8.4.0.0,3,7]tetradeca-1(14),3(7),5,10,12-pentaene-9-carbonylphenyl)methyl]pyrrolidine-1-carboxamide

1-(2-Methyl-4-4-methyl-2,4,5,9-tetraazatricyclo[8.4.0.0,3,7]tetradeca-1(14),3(7),5,10,12-pentaene-9-carbonylphenyl)methanamine (6) (5.3 mg, 15.28 μmol), 1-(1H-imidazole-1-carbonyl)-1H-imidazole (4.95 mg, 30.56 μmol) and ethylbis(propan-2-yl)amine (20 mg, 152.8 μmol) were dissolved in DMF (5 ml) and stirred at 15° C. for 3 h. (2S,4R)-4-[(4-hydroxyphenyl)methoxy]-N,N-dimethylpyrrolidine-2-carbothioamide (5) (6 mg, 21.4 μmol) was added to the mixture and it was stirred at rt for 16 h. LCMS from the reaction mixture showed 66% of the desired product. The reaction mixture was purified by HPLC (SYSTEM 0-2-8 min 7-15-35% H2O/ACN 30 ml/min (loading pump 4 ml ACN), target mass 653 column: Chromatorex C18 SMB100-5T 100*19 mm, 5 microM) to afford (2S,4R)-2-(dimethylcarbamothioyl)-4-[(4-hydroxyphenyl)methoxy]-N-[(2-methyl-4-4-methyl-2,4,5,9-tetraazatricyclo[8.4.0.0,3,7]tetradeca-1(14),3(7),5,10,12-pentaene-9-carbonylphenyl)methyl]pyrrolidine-1-carboxamide, LIT-003-07. Yield: 5.0 mg, 50.1%; Appearance: Brown solid; HPLC purity: 100%; LCMS Calculated for C₃₅H₃₉N₇O₄S: 654.32; Observed: 654.4[M+H]⁺.

### The synthesis of (2S,4R)-4-[(3-carbamoylphenyl)methoxy]-2-(dimethylcarbamothioyl)-N-[(2-methyl-4-4-methyl-2,4,5,9-tetraazatricyclo[8.4.0.0,3,7]tetradeca-1(14),3(7),5,10,12-pentaene-9-carbonylphenyl)methyl]pyrrolidine-1-carboxamide (LIT-003-08)

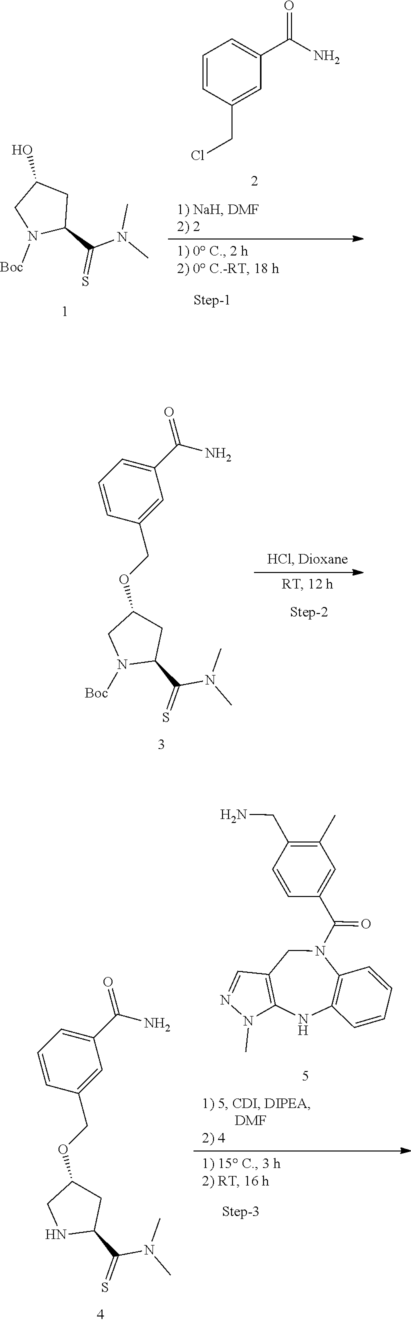 [SMILES: CN(C)C(=S)[C@@H]1C[C@@H](O)CN1C(=O)OC(C)(C)C ; NC(=O)c1cccc(CCl)c1 ; CN(C)C(=S)[C@@H]1C[C@@H](OCc2cccc(C(N)=O)c2)CN1C(=O)OC(C)(C)C ; CN(C)C(=S)[C@@H]1C[C@@H](OCc2cccc(C(N)=O)c2)CN1 ; Cc1cc(C(=O)N2Cc3cnn(C)c3Nc3ccccc32)ccc1CN]

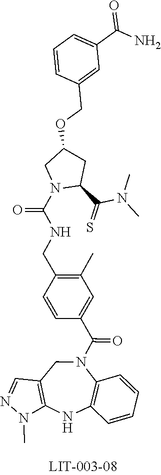 [SMILES: Cc1cc(C(=O)N2Cc3cnn(C)c3Nc3ccccc32)ccc1CNC(=O)N1C[C@H](OCc2cccc(C(N)=O)c2)C[C@H]1C(=S)N(C)C]

### Step 1. Synthesis of tert-butyl (2S,4R)-4-[(3-carbamoylphenyl)methoxy]-2-(dimethylcarbamothioyl)pyrrolidine-1-carboxylate

To a stirred suspension of tert-butyl (2S,4R)-2-(dimethylcarbamothioyl)-4-hydroxypyrrolidine-1-carboxylate (1) (600 mg, 2.19 mmol) in DMF at 0° C. was added sodium hydride (210 mg, 8.75 mmol) and the mixture was stirred at 0° C. for 2 h, then 3-(chloromethyl)benzamide (2) (370 mg, 2.19 mmol) was added and the reaction mixture was stirred at 0° C. for another 2 h, allowed to warm to rt and stirred for 16 h. LCMS from the reaction mixture showed 38% of the desired product. The reaction mixture was concentrated and purified by HPLC (SYSTEM 0-2-8 min 23-30-60% H2O/MEOH/, 30 ml\min (loading pump 4 ml MeOH), target mass 407 column: Chromatorex 100*19 mm, 5 microM) to afford tert-butyl (2S,4R)-4-[(3-carbamoylphenyl)methoxy]-2-(dimethylcarbamothioyl)pyrrolidine-1-carboxylate (3) (200 mg, 98.0% purity, 480.95 μmol, 22% yield).

### Step 2. Synthesis of 3-([(3R,5S)-5-(dimethylcarbamothioyl)pyrrolidin-3-yl]oxymethyl)benzamide hydrochloride

Tert-butyl (2S,4R)-4-[(3-carbamoylphenyl)methoxy]-2-(dimethylcarbamothioyl)pyrrolidine-1-carboxylate (3) (200 mg, 490.77 μmol) was dissolved in 4M Dioxane-HCl (10 ml) and the resulting mixture was stirred at rt for 16 h. LCMS from the reaction mixture showed 71% of the desired product. The reaction mixture was concentrated to obtain 3-([(3R,5S)-5-(dimethylcarbamothioyl)pyrrolidin-3-yl]oxymethyl)benzamide hydrochloride (4) (140 mg, 71.0% purity, 289.06 μmol, 58.8% yield)

### Step 3. Synthesis of (2S,4R)-4-[(3-carbamoylphenyl)methoxy]-2-(dimethylcarbamothioyl)-N-[(2-methyl-4-4-methyl-2,4,5,9-tetraazatricyclo[8.4.0.0,3,7]tetradeca-1(14),3(7),5,10,12-pentaene-9-carbonylphenyl)methyl]pyrrolidine-1-carboxamide

1-(2-Methyl-4-4-methyl-2,4,5,9-tetraazatricyclo[8.4.0.0,3,7]tetradeca-1(14),3(7),5,10,12-pentaene-9-carbonylphenyl)methanamine (5) (101 mg, 290.83 μmol), 1-(1H-imidazole-1-carbonyl)-1H-imidazole (94 mg, 581.65 μmol) and ethylbis(propan-2-yl)amine (375.61 mg, 2.91 mmol) were dissolved in DMF (5 ml) and stirred at 15° C. for 3 h. 3-([(3R,5S)-5-(dimethylcarbamothioyl)pyrrolidin-3-yl]oxymethyl)benzamide hydrochloride (4) (140 mg, 407.13 μmol) was added to the mixture and it was stirred at rt for 16 h. LCMS from the reaction mixture showed 88% of the desired product. The reaction mixture was purified by HPLC (SYSTEM 0-2-8 min 33-40-100% H2O/ACN 30 ml/min (loading pump 4 ml ACN), target mass 680 column: XBRIDGE BEH C18 SMB100-5T 100*19 mm, 5 microM) to afford (2S,4R)-4-[(3-carbamoylphenyl)methoxy]-2-(dimethylcarbamothioyl)-N-[(2-methyl-4-4-methyl-2,4,5,9-tetraazatricyclo[8.4.0.0,3,7]tetradeca-1(14),3(7),5,10,12-pentaene-9-carbonylphenyl)methyl]pyrrolidine-1-carboxamide, LIT-003-08. Yield: 141 mg, 71.2%; Appearance: Yellow oil; HPLC purity: 100%; LCMS Calculated for C₃₆H₄₀N₈O₄S: 681.33; Observed: 681.2[M+H]⁺.

### The synthesis of (2S,4R)-2-(dimethylcarbamothioyl)-N-[(2-methyl-4-4-methyl-2,4,5,9-tetraazatricyclo[8.4.0.0,3,7]tetradeca-1(14),3(7),5,10,12-pentaene-9-carbonylphenyl)methyl]-4-[(3-sulfamoylphenyl)methoxy]pyrrolidine-1-carboxamide (LIT-003-09)

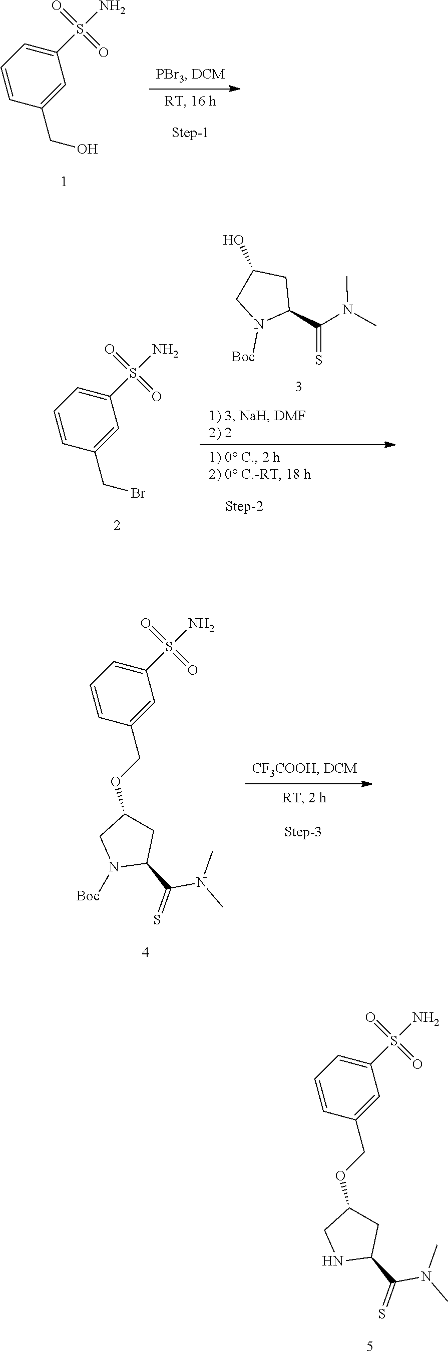 [SMILES: NS(=O)(=O)c1cccc(CO)c1 ; NS(=O)(=O)c1cccc(CBr)c1 ; CN(C)C(=S)[C@@H]1C[C@@H](O)CN1C(=O)OC(C)(C)C ; CN(C)C(=S)[C@@H]1C[C@@H](OCc2cccc(S(N)(=O)=O)c2)CN1C(=O)OC(C)(C)C ; CN(C)C(=S)[C@@H]1C[C@@H](OCc2cccc(S(N)(=O)=O)c2)CN1]

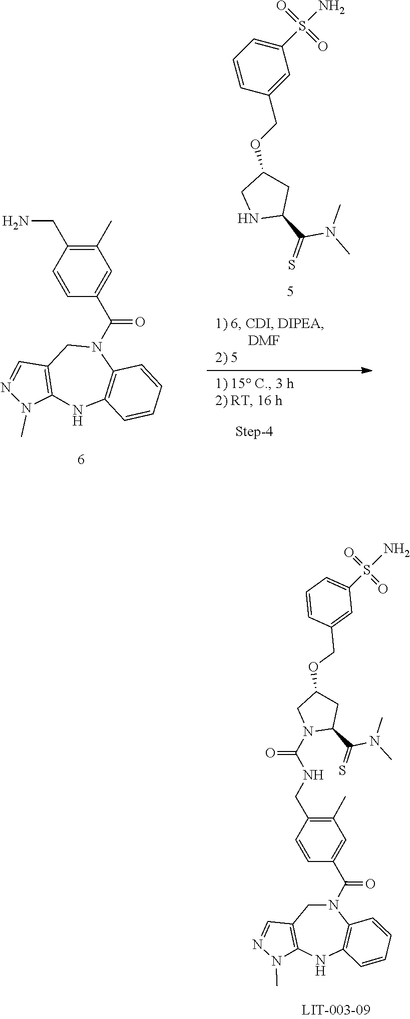 [SMILES: Cc1cc(C(=O)N2Cc3cnn(C)c3Nc3ccccc32)ccc1CN ; CN(C)C(=S)[C@@H]1C[C@@H](OCc2cccc(S(N)(=O)=O)c2)CN1 ; Cc1cc(C(=O)N2Cc3cnn(C)c3Nc3ccccc32)ccc1CNC(=O)N1C[C@H](OCc2cccc(S(N)(=O)=O)c2)C[C@H]1C(=S)N(C)C]

### Step 1. Synthesis of 3-(bromomethyl)benzene-1-sulfonamide

3-(Hydroxymethyl)benzene-1-sulfonamide (1) (2.0 g, 10.69 mmol) was dissolved in DCM and tribromophosphane (3.44 g, 12.83 mmol) was added at rt. The resulting mixture was stirred at rt for 16 h. Water was added carefully and the organic phase was separated and washed with brine, dried over disodium sulfate and concentrated to obtain 3-(bromomethyl)benzene-1-sulfonamide (2) (750 mg, 94.0% purity, 2.82 mmol, 26.4% yield).

### Step 2. Synthesis of tert-butyl (2S,4R)-2-(dimethylcarbamothioyl)-4-[(3-sulfamoylphenyl)methoxy]pyrrolidine-1-carboxylate

To a stirred solution of tert-butyl (2S,4R)-2-(dimethylcarbamothioyl)-4-hydroxypyrrolidine-1-carboxylate (3) (460 mg, 1.68 mmol) in DMF at 0° C. was added sodium hydride (161 mg, 6.71 mmol) and the mixture was stirred at 0° C. for 2 h, then 3-(bromomethyl)benzene-1-sulfonamide (2) (418 mg, 1.68 mmol) was added and the reaction mixture was stirred at 0° C. for another 2 h, allowed to warm to rt and stirred for 16 h. LCMS from the reaction mixture showed 31% of the desired product. The reaction mixture was concentrated and purified by HPLC (SYSTEM 0-2-8 min 0-0-25% H2O/ACN/0.1% NH4OH 30 ml/min (loading pump 4 ml ACN), target mass 443 column: XBRIDGE BEH C18 SMB100-5T 100*19 mm, 5 microM) to afford tert-butyl (2S,4R)-2-(dimethylcarbamothioyl)-4-[(3-sulfamoylphenyl)methoxy]pyrrolidine-1-carboxylate (4) (52.0 mg, 96.0% purity, 112.54 μmol, 6.7% yield)

### Step 3. Synthesis of (2S,4R)-N,N-dimethyl-4-[(3-sulfamoylphenyl)methoxy]pyrrolidine-2-carbothioamide

Tert-butyl (2S,4R)-2-(dimethylcarbamothioyl)-4-[(3-sulfamoylphenyl)methoxy]pyrrolidine-1-carboxylate (4) (52 mg, 117.23 μmol) was dissolved in DCM (2 ml) and TFA (1 ml) was added to the mixture at 0° C. The resulting mixture was stirred at rt for 2 h. LCMS from the reaction mixture indicated 84% of the desired product. The mixture was concentrated to obtain (2S,4R)-N,N-dimethyl-4-[(3-sulfamoylphenyl)methoxy]pyrrolidine-2-carbothioamide (5) (40 mg, 84.0% purity, 97.83 μmol, 83.4% yield) as a TFA salt.

### Step 4. Synthesis of (2S,4R)-2-(dimethylcarbamothioyl)-N-[(2-methyl-4-4-methyl-2,4,5,9-tetraazatricyclo[8.4.0.0,3,7]tetradeca-1(14),3(7),5,10,12-pentaene-9-carbonylphenyl)methyl]-4-[(3-sulfamoylphenyl)methoxy]pyrrolidine-1-carboxamide

1-(2-Methyl-4-4-methyl-2,4,5,9-tetraazatricyclo[8.4.0.0,3,7]tetradeca-1(14),3(7),5,10,12-pentaene-9-carbonylphenyl)methanamine (6) (29 mg, 83.14 μmol), 1-(1H-imidazole-1-carbonyl)-1H-imidazole (27 mg, 166.28 μmol) and ethylbis(propan-2-yl)amine (107 mg, 831.39 μmol) were dissolved in DMF (5 ml) and stirred at 15° C. for 3 h. (2S,4R)-N,N-dimethyl-4-[(3-sulfamoylphenyl)methoxy]pyrrolidine-2-carbothioamide (5) (40 mg, 116.46 μmol) was added to the mixture and it was stirred at RT for 16 h. LCMS from the reaction mixture showed 89% of the desired product. The reaction mixture was purified by HPLC (SYSTEM 0-2-8 min 13-20-40% H2O/ACN 30 ml/min (loading pump 4 ml ACN), target mass 716 column: CHROMATOREX C18 SMB100-5T 100*19 mm, 5 microM) to afford (2S,4R)-2-(dimethylcarbamothioyl)-N-[(2-methyl-4-4-methyl-2,4,5,9-tetraazatricyclo[8.4.0.0,3,7]tetradeca-1(14),3(7),5,10,12-pentaene-9-carbonylphenyl)methyl]-4-[(3-sulfamoylphenyl)methoxy]pyrrolidine-1-carboxamide, LIT-003-09. Yield: 32.0 mg, 53.7%; Appearance: Brown solid; HPLC purity: 100%; LCMS Calculated for C₃₄H₃₉N₈O₅S₂: 717.29; Observed: 717.0[M+H]⁺.

### The synthesis of 5-chloro-N-(3-chloro-5-cyclopropylphenyl)-2-methoxy-4-methylbenzene-1-sulfonamide (LIT-003-10)

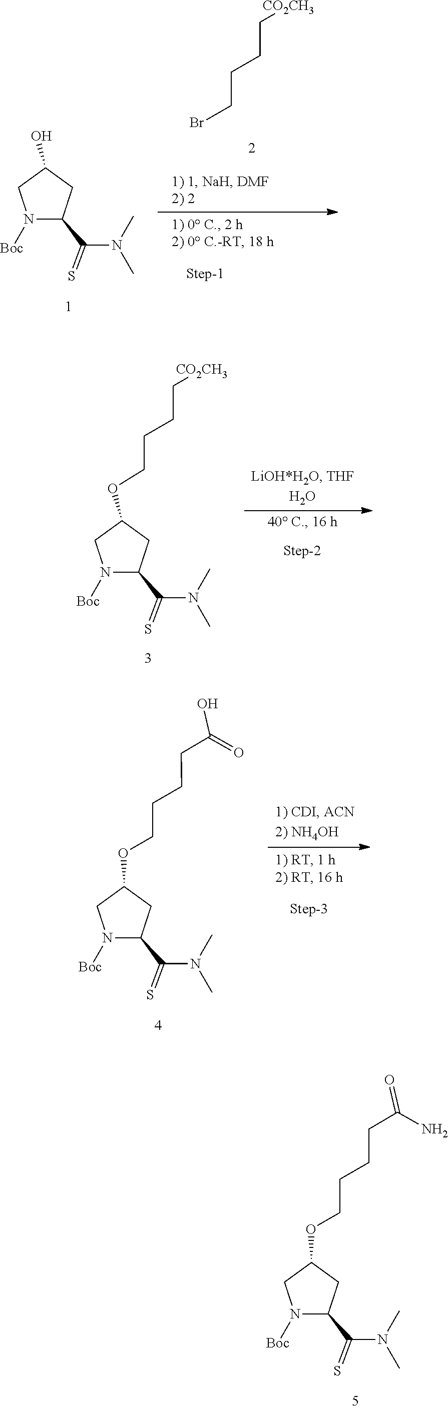 [SMILES: COC(=O)CCCCBr ; CN(C)C(=S)[C@@H]1C[C@@H](O)CN1C(=O)OC(C)(C)C ; COC(=O)CCCCO[C@@H]1C[C@@H](C(=S)N(C)C)N(C(=O)OC(C)(C)C)C1 ; CN(C)C(=S)[C@@H]1C[C@@H](OCCCCC(=O)O)CN1C(=O)OC(C)(C)C ; CN(C)C(=S)[C@@H]1C[C@@H](OCCCCC(N)=O)CN1C(=O)OC(C)(C)C]

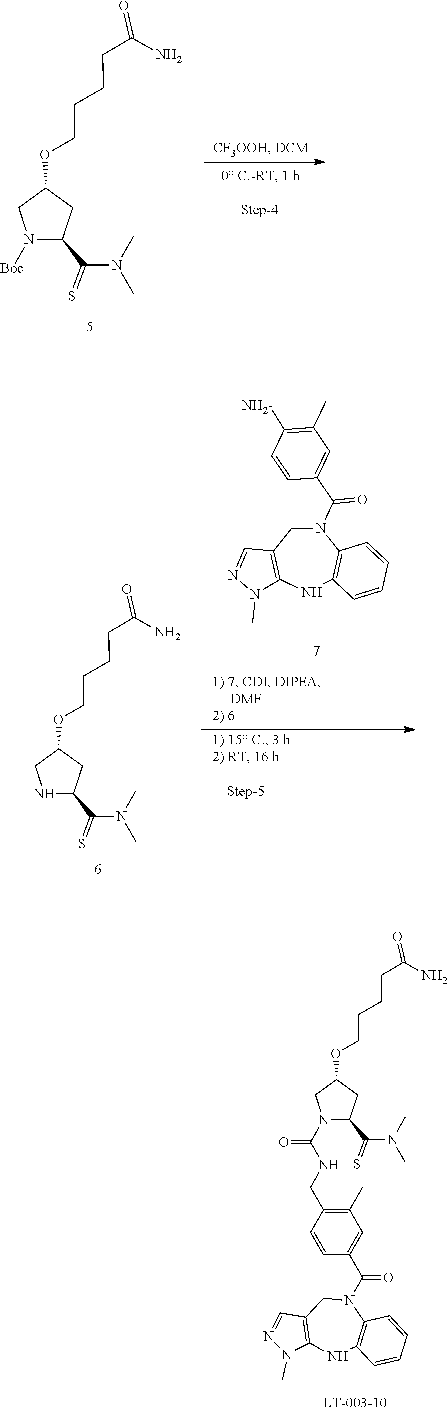 [SMILES: CN(C)C(=S)[C@@H]1C[C@@H](OCCCCC(N)=O)CN1C(=O)OC(C)(C)C ; CN(C)C(=S)[C@@H]1C[C@@H](OCCCCC(N)=O)CN1 ; Cc1cc(C(=O)N2Cc3cnn(C)c3Nc3ccccc32)ccc1[NH-] ; Cc1cc(C(=O)N2Cc3cnn(C)c3Nc3ccccc32)ccc1CNC(=O)N1C[C@H](OCCCCC(N)=O)C[C@H]1C(=S)N(C)C]

### Step 1. Synthesis of tert-butyl (2S,4R)-2-(dimethylcarbamothioyl)-4-[(5-methoxy-5-oxopentyl)oxy]pyrrolidine-1-carboxylate

To a stirred solution of tert-butyl (2S,4R)-2-(dimethylcarbamothioyl)-4-hydroxypyrrolidine-1-carboxylate (1) (600 mg, 2.19 mmol) in DMF at 0° C. was added sodium hydride (262 mg, 10.94 mmol) and the mixture was stirred at 0° C. for 2 h, then methyl 5-bromopentanoate (2) (424 mg, 2.19 mmol) was added and the reaction mixture was stirred at 0° C. for another 2 h, allowed to warm to rt and stirred for 16 h. LCMS from the reaction mixture showed 37% of the desired product. The reaction mixture was concentrated and purified by HPLC (SYSTEM 0-2-8 min 17-25-45% H2O/ACN 30 ml/min (loading pump 4 ml ACN), target mass 388 column: CHROMATOREX C18 100*19 mm, 5 microM, 130A) to afford tert-butyl (2S,4R)-2-(dimethylcarbamothioyl)-4-[(5-methoxy-5-oxopentyl)oxy]pyrrolidine-1-carboxylate (3) (190 mg, 96.0% purity, 469.47 μmol, 21.5% yield).

### Step 2. Synthesis of 5-[(3R,5S)-1-[(tert-butoxy)carbonyl]-5-(dimethylcarbamothioyl)pyrrolidin-3-yl]oxypentanoic acid

Tert-butyl (2S,4R)-2-(dimethylcarbamothioyl)-4-[(5-methoxy-5-oxopentyl)oxy]pyrrolidine-1-carboxylate (3) (80.0 mg, 205.91 μmol) and lithium(1+) ion hydrate hydroxide (17.31 mg, 411.75 μmol) were mixed in THF-H₂O solution and stirred at 40° C. for 16 h. LCMS from the reaction mixture indicated 99% of the desired product. The mixture was concentrated, diluted with water, acidified with NaHSO4 solution, and extracted with EtOAc. The organic layer was washed with water, dried over NaSO4 and evaporated to obtain 5-[(3R,5S)-1-[(tert-butoxy)carbonyl]-5-(dimethylcarbamothioyl)pyrrolidin-3-yl]oxypentanoic acid (4) (60.0 mg, 99.0% purity, 158.61 μmol, 77% yield).

### Step 3. Synthesis of 5-chloro-N-(3-chloro-5-cyclopropylphenyl)-2-methoxy-4-methylbenzene-1-sulfonamide

5-[(3R,5S)-1-[(tert-butoxy)carbonyl]-5-(dimethylcarbamothioyl)pyrrolidin-3-yl]oxypentanoic acid (4) (60 mg, 160.35 μmol) and 1-(1H-imidazole-1-carbonyl)-1H-imidazole (31. mg, 192.44 μmol) were mixed in ACN and stirred at rt for 1 h. After 1 h amine hydrate (17 mg, 481.11 μmol) was added and the mixture was stirred at rt for 16 h. LCMS from the reaction mixture indicated 99% of the desired product. The mixture was concentrated and the residue was dissolved in DCM, washed with NaHSO₄, dried over Na₂SO₄ and evaporated to obtain tert-butyl (2S,4R)-4-(4-carbamoylbutoxy)-2-(dimethylcarbamothioyl)pyrrolidine-1-carboxylate (5) (40.0 mg, 99.0% purity, 106.02 μmol, 66.1% yield).

### Step 4. Synthesis of 5-[(3R,5S)-5-(dimethylcarbamothioyl)pyrrolidin-3-yl]oxypentanamide

Tert-butyl (2S,4R)-4-(4-carbamoylbutoxy)-2-(dimethylcarbamothioyl)pyrrolidine-1-carboxylate (5) (40.0 mg, 107.09 μmol) was dissolved in DCM (2 ml) and TFA (1 ml) was added to the mixture at 0° C. The resulting mixture was stirred at rt for 2 h. LCMS from the reaction mixture indicated 100% of the desired product. The mixture was concentrated to obtain 5-[(3R,5S)-5-(dimethylcarbamothioyl)pyrrolidin-3-yl]oxypentanamide (6) (30.0 mg, 109.73 μmol, 102.4% yield) as a TFA salt.

### Step 5. Synthesis of afford (2S,4R)-4-(4-carbamoylbutoxy)-2-(dimethylcarbamothioyl)-N-[(2-methyl-4-4-methyl-2,4,5,9-tetraazatricyclo[8.4.0.0,3,7]tetradeca-1(14),3(7),5,10,12-pentaene-9-carbonylphenyl)methyl]pyrrolidine-1-carboxamide

1-(2-Methyl-4-4-methyl-2,4,5,9-tetraazatricyclo[8.4.0.0,3,7]tetradeca-1(14),3(7),5,10,12-pentaene-9-carbonylphenyl)methanamine (7) (27.21 mg, 78.39 μmol), 1-(1H-imidazole-1-carbonyl)-1H-imidazole (25.41 mg, 156.77 μmol) and ethylbis(propan-2-yl)amine (101.24 mg, 783.86 μmol) were dissolved in DMF (5 ml) and stirred at 15° C. for 3 h. 5-[(3R,5S)-5-(dimethylcarbamothioyl)pyrrolidin-3-yl]oxypentanamide (6) (30.0 mg, 109.73 μmol) was added to the mixture and it was stirred at rt for 16 h. LCMS from the reaction mixture showed 84% of the desired product. The reaction mixture was purified by HPLC (SYSTEM 0-2-8 min 23-30-55% H2O/ACN 30 ml/min (loading pump 4 ml ACN), target mass 646 column: Chromatorex C18 SMB100-5T 100*19 mm, 5 microM) to afford (2S,4R)-4-(4-carbamoylbutoxy)-2-(dimethylcarbamothioyl)-N-[(2-methyl-4-4-methyl-2,4,5,9-tetraazatricyclo[8.4.0.0,3,7]tetradeca-1(14),3(7),5,10,12-pentaene-9-carbonylphenyl)methyl]pyrrolidine-1-carboxamide, LIT-003-10. Yield: 15.6 mg, 30.2%; Appearance: White solid; HPLC purity: 98.78%; LCMS Calculated for C₃₃H₄₂N₈O₄S: 647.35; Observed: 647.2[M+H]⁺.

### The synthesis of (2S,4R)-2-(dimethylcarbamothioyl)-4-(3-[2-(2-hydroxyethoxy)ethoxy]phenylmethoxy)-N-[(2-methyl-4-4-methyl-2,4,5,9-tetraazatricyclo[8.4.0.0,3,7]tetradeca-1(14),3(7),5,10,12-pentaene-9-carbonylphenyl)methyl]pyrrolidine-1-carboxamide (LIT-003-11)

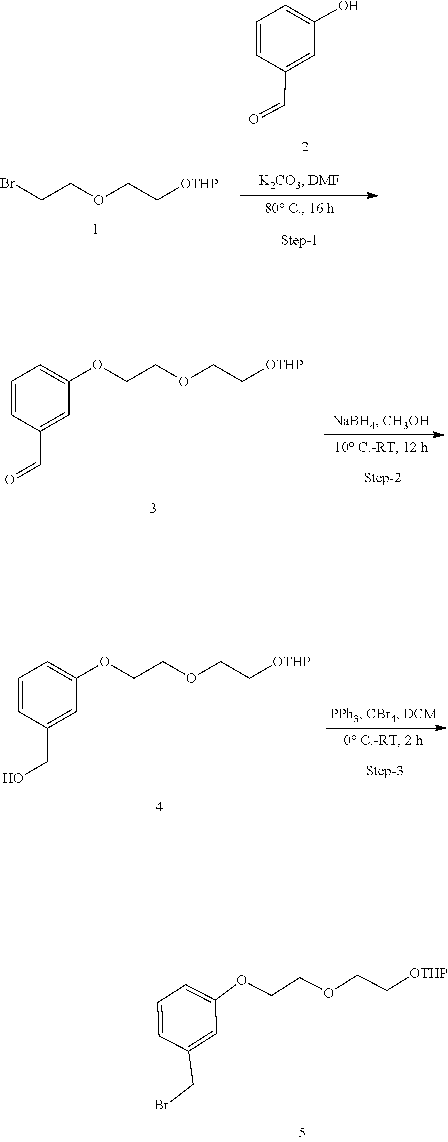

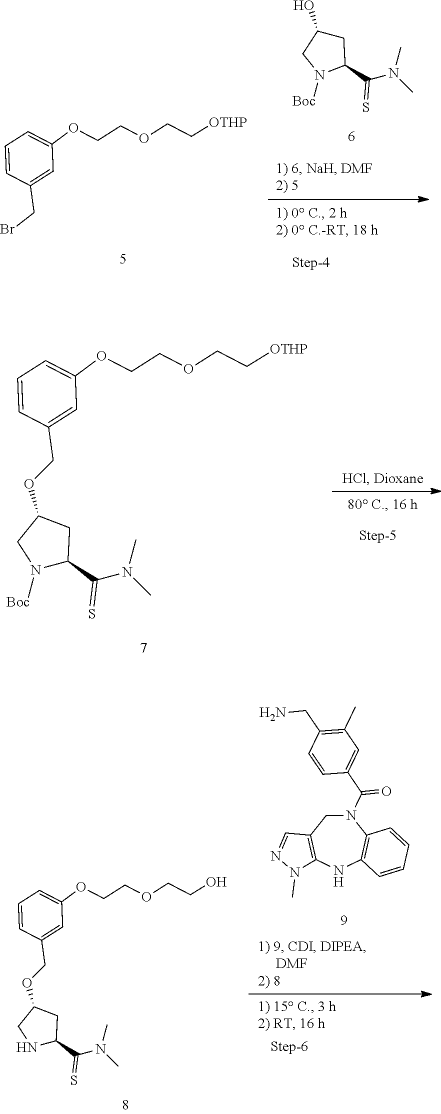

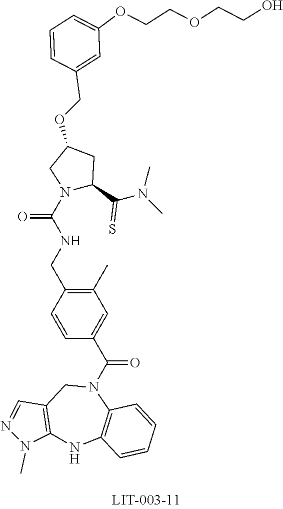 [SMILES: Cc1cc(C(=O)N2Cc3cnn(C)c3Nc3ccccc32)ccc1CNC(=O)N1C[C@H](OCc2cccc(OCCOCCO)c2)C[C@H]1C(=S)N(C)C]

### Step 1. Synthesis of 3-2-[2-(oxan-2-yloxy)ethoxy]ethoxybenzaldehyde

2-[2-(2-Bromoethoxy)ethoxy]oxane (1) (800 mg, 3.17 mmol), 3-hydroxybenzaldehyde (2) (352 mg, 2.88 mmol) and dipotassium carbonate (597 mg, 4.33 mmol) were mixed in DMF and stirred at 80° C. for 16 h. LCMS from the reaction mixture indicated 93% of the desired product. The mixture was poured into water, extracted with EtOAc, the organic layer was washed with water and brine, dried over Na₂SO₄ and evaporated to obtain 3-2-[2-(oxan-2-yloxy)ethoxy]ethoxybenzaldehyde (3) (840 mg, 93.0% purity, 2.65 mmol, 92% yield).

### Step 2. Synthesis of (3-2-[2-(oxan-2-yloxy)ethoxy]ethoxyphenyl)methanol

To a stirred solution of 3-2-[2-(oxan-2-yloxy)ethoxy]ethoxybenzaldehyde (3) (840 mg, 2.85 mmol) in MeOH at 10° C. was added sodium borohydride (271 mg, 7.14 mmol) portionwise and the mixture was stirred at rt for 16 h. LCMS from the reaction mixture indicated 94% of the desired product. The mixture was poured into saturated NH₄Cl solution, extracted with EtOAc, the organic layer was washed with water and brine, dried over Na₂SO₄ and evaporated to obtain (3-2-[2-(oxan-2-yloxy)ethoxy]ethoxyphenyl)methanol (4) (830 mg, 94.0% purity, 2.63 mmol, 92.2% yield).

### Step 3. Synthesis of 2-(2-2-[3-(bromomethyl)phenoxy]ethoxyethoxy)oxane

To a stirred solution of (3-2-[2-(oxan-2-yloxy)ethoxy]ethoxyphenyl)methanol (4) (830 mg, 2.8 mmol) in DCM at 0° C. was added tribromophosphane (825 mg, 3.08 mmol, 290.0 μl, 1.1 equiv) dropwise and the mixture was stirred at rt for 2 h. After 2 h the mixture was poured into water, the organic layer was washed with saturated NaHCO₃ solution, dried over Na₂SO₄ and evaporated to obtain 2-(2-2-[3-(bromomethyl)phenoxy]ethoxyethoxy)oxane (5) (800 mg, 80.0% purity, 1.78 mmol, 63.6% yield). NMR of the product was 80% pure.

### Step 4. Synthesis of tert-butyl (2S,4R)-2-(dimethylcarbamothioyl)-4-[(3-2-[2-(oxan-2-yloxy)ethoxy]ethoxyphenyl)methoxy]pyrrolidine-1-carboxylate

To a stirred solution of tert-butyl (2S,4R)-2-(dimethylcarbamothioyl)-4-hydroxypyrrolidine-1-carboxylate (6) (611 mg, 2.23 mmol) in DMF at 0° C. was added sodium hydride (214 mg, 8.91 mmol) and the mixture was stirred at 0° C. for 2 h, then 2-(2-2-[3-(bromomethyl)phenoxy]ethoxyethoxy)oxane (7) (800 mg, 2.23 mmol) was added and the reaction mixture was stirred at 0° C. for another 2 h, allowed to warm to rt and stirred for 16 h. LCMS from the reaction mixture showed 24% of the desired product. The reaction mixture was purified by HPLC (SYSTEM 0-2-8 min 33-40-60% H₂O/ACN 30 ml/min (loading pump 4 ml ACN), target mass 468 column: CHROMATOREX C18 100*19 mm, 5 microM, 130A) to afford tert-butyl (2S,4R)-2-(dimethylcarbamothioyl)-4-[(3-2-[2-(oxan-2-yloxy)ethoxy]ethoxyphenyl)methoxy]pyrrolidine-1-carboxylate (7) (93 mg, 99.0% purity, 166.58 μmol, 7.5% yield).

### Step 5. Synthesis of (2S,4R)-4-(3-[2-(2-hydroxyethoxy)ethoxy]phenylmethoxy)-N,N-dimethylpyrrolidine-2-carbothioamide hydrochloride

Tert-butyl (2S,4R)-2-(dimethylcarbamothioyl)-4-[(3-2-[2-(oxan-2-yloxy)ethoxy]ethoxyphenyl)methoxy]pyrrolidine-1-carboxylate (7) (60 mg, 108.55 μmol) was dissolved in 4M Dioxane-HCl solution and stirred at 50° C. for 12 h. LCMS from the reaction mixture indicated 79% of the desired product. The mixture was concentrated to obtain (2S,4R)-4-(3-[2-(2-hydroxyethoxy)ethoxy]phenylmethoxy)-N,N-dimethylpyrrolidine-2-carbothioamide hydrochloride (8) (40 mg, 79.0% purity, 78.03 μmol, 71.8% yield).

### Step 6. Synthesis of (2S,4R)-2-(dimethylcarbamothioyl)-4-(3-[2-(2-hydroxyethoxy)ethoxy]phenylmethoxy)-N-[(2-methyl-4-4-methyl-2,4,5,9-tetraazatricyclo[8.4.0.0,3,7]tetradeca-1(14),3(7),5,10,12-pentaene-9-carbonylphenyl)methyl]pyrrolidine-1-carboxamide

1-(2-Methyl-4-4-methyl-2,4,5,9-tetraazatricyclo[8.4.0.0,3,7]tetradeca-1(14),3(7),5,10,12-pentaene-9-carbonylphenyl)methanamine (9) (24 mg, 70.49 μmol), 1-(1H-imidazole-1-carbonyl)-1H-imidazole (23 mg, 140.99 μmol) and ethylbis(propan-2-yl)amine (91 mg, 704.95 μmol) were dissolved in DMF (5 ml) and stirred at 15° C. for 3 h. (2S,4R)-4-(3-[2-(2-hydroxyethoxy)ethoxy]phenylmethoxy)-N,N-dimethylpyrrolidine-2-carbothioamide hydrochloride (8) (40 mg, 98.78 μmol) was added to the mixture and it was stirred at rt for 16 h. LCMS from the reaction mixture showed 61% of the desired product. The reaction mixture was purified by HPLC (SYSTEM 0-3 min 13-20-40% H₂O/ACN 30 ml/min (loading pump 4 ml ACN), target mass 741 column: Chromatorex C18 SMB100-5T 100*19 mm, 5 microM) to afford (2S,4R)-2-(dimethylcarbamothioyl)-4-(3-[2-(2-hydroxyethoxy)ethoxy]phenylmethoxy)-N-[(2-methyl-4-4-methyl-2,4,5,9-tetraazatricyclo[8.4.0.0,3,7]tetradeca-1(14),3(7),5,10,12-pentaene-9-carbonylphenyl)methyl]pyrrolidine-1-carboxamide, LIT-003-11. Yield: 29 mg, 55.4%; Appearance: Beige solid; HPLC purity: 100%; LCMS Calculated for C₃₉H₄₇N₇O₆S: 742.38; Observed: 743.2 [M+H]⁺.

### The synthesis of (2S,4R)-2-(dimethylcarbamothioyl)-4-[(1H-indol-6-yl)methoxy]-N-[(2-methyl-4-4-methyl-2,4,5,9-tetraazatricyclo[8.4.0.0,3,7]tetradeca-1(14),3(7),5,10,12-pentaene-9-carbonylphenyl)methyl]pyrrolidine-1-carboxamide (LIT-003-12)

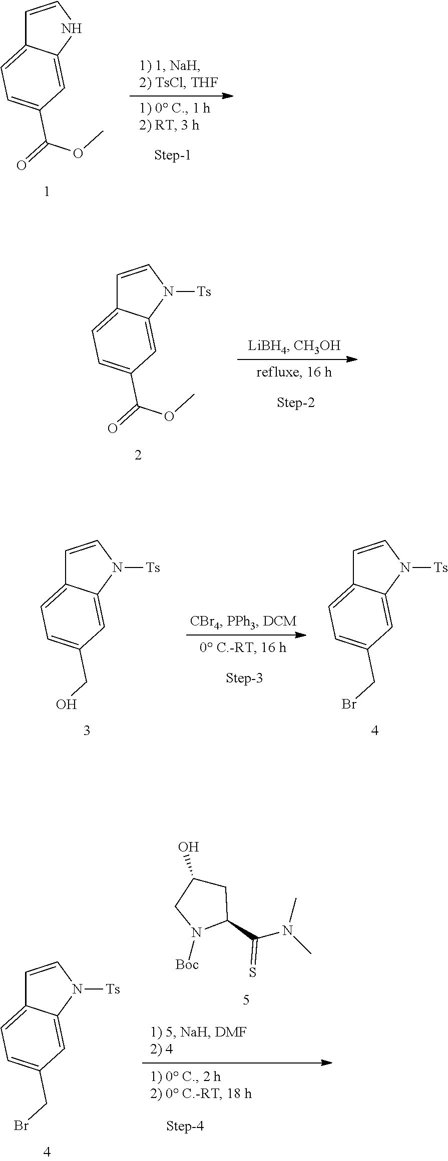 [SMILES: COC(=O)c1ccc2cc[nH]c2c1 ; COC(=O)c1ccc2ccn(S(=O)(=O)c3ccc(C)cc3)c2c1 ; Cc1ccc(S(=O)(=O)n2ccc3ccc(CO)cc32)cc1 ; Cc1ccc(S(=O)(=O)n2ccc3ccc(CBr)cc32)cc1 ; Cc1ccc(S(=O)(=O)n2ccc3ccc(CBr)cc32)cc1 ; CN(C)C(=S)[C@@H]1C[C@@H](O)CN1C(=O)OC(C)(C)C]

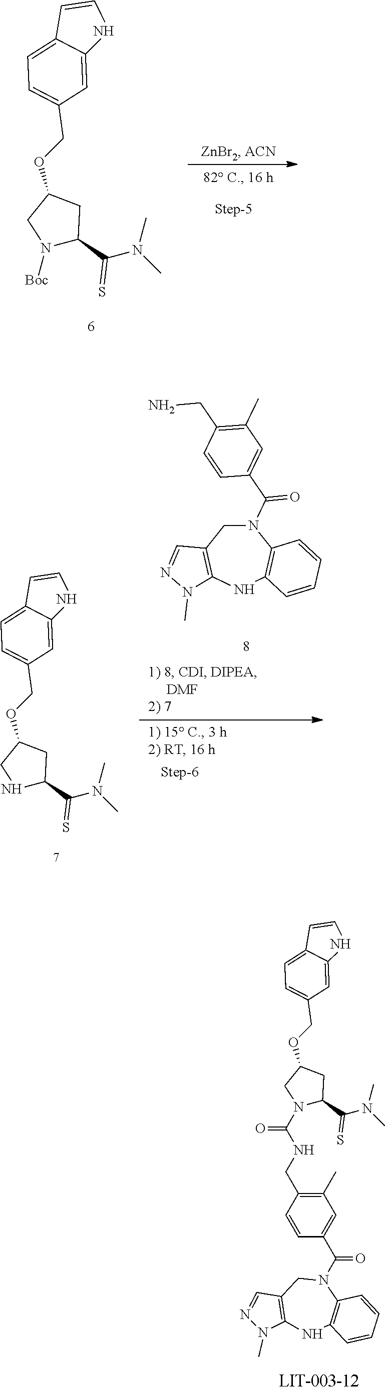 [SMILES: CN(C)C(=S)[C@@H]1C[C@@H](OCc2ccc3cc[nH]c3c2)CN1C(=O)OC(C)(C)C ; CN(C)C(=S)[C@@H]1C[C@@H](OCc2ccc3cc[nH]c3c2)CN1 ; Cc1cc(C(=O)N2Cc3cnn(C)c3Nc3ccccc32)ccc1CN ; Cc1cc(C(=O)N2Cc3cnn(C)c3Nc3ccccc32)ccc1CNC(=O)N1C[C@H](OCc2ccc3cc[nH]c3c2)C[C@H]1C(=S)N(C)C]

### Step 1. Synthesis of methyl 1-(4-methylbenzenesulfonyl)-1H-indole-6-carboxylate

To a stirred solution of methyl 1H-indole-6-carboxylate (1) (1.79 g, 10.22 mmol) at 0° C. was added sodium hydride (491 mg, 20.45 mmol) and the mixture was stirred at 0° C. for 1 h. After 1 h 4-methylbenzene-1-sulfonyl chloride (2.53 g, 13.29 mmol) was added and the mixture was allowed to warm to rt and stirred at rt for 3 h. After 3 h the mixture was poured into water, extracted with EtOAc, combined organic phase was washed with brine, dried over Na₂SO₄ and evaporated to obtain methyl 1-(4-methylbenzenesulfonyl)-1H-indole-6-carboxylate (2) (3.4 g, 80.0% purity, 8.26 mmol, 80.8% yield).

### Step 2. Synthesis of 1-(4-methylbenzenesulfonyl)-1H-indol-6-yl]methanol

To a stirred solution of methyl 1-(4-methylbenzenesulfonyl)-1H-indole-6-carboxylate (2) (3.39 g, 10.3 mmol) at 10° C. was added lithium(1+) ion borohydride (454 mg, 20.61 mmol) portionwise, the reaction mixture was allowed to warm to rt and heated to reflux for 16 h. After 16 h the mixture was cooled to rt, poured into saturated NH₄Cl solution, extracted with EtOAc combined organic phase was washed with brine, dried over Na₂S_{O4} and evaporated to obtain [1-(4-methylbenzenesulfonyl)-1H-indol-6-yl]methanol (3) (2.6 g, 86.0% purity, 7.42 mmol, 72% yield).

### Step 3. Synthesis of 6-(bromomethyl)-1-(4-methylbenzenesulfonyl)-1H-indole

To a stirred solution of [1-(4-methylbenzenesulfonyl)-1H-indol-6-yl]methanol (3) (2.4 g, 7.97 mmol) in DCM at 0° C. was added triphenylphosphane (3.13 g, 11.96 mmol) and the mixture was stirred at 0° C. for 10 min. After 10 min tetrabromomethane (3.92 g, 11.96 mmol) was added and the mixture was left to stirred at rt for 16 h. After 16 h the mixture was concentrated and the obtained residue was purified by FC (system EtOAc-Hexane) to afford 6-(bromomethyl)-1-(4-methylbenzenesulfonyl)-1H-indole (4) (2.0 g, 95.0% purity, 5.22 mmol, 65.4% yield).

### Step 4. Synthesis of tert-butyl (2S,4R)-2-(dimethylcarbamothioyl)-4-[(1H-indol-6-yl)methoxy]pyrrolidine-1-carboxylate

To a stirred solution of tert-butyl (2S,4R)-2-(dimethylcarbamothioyl)-4-hydroxypyrrolidine-1-carboxylate (5) (753 mg, 2.74 mmol) in DMF at 0° C. was added sodium hydride (263 mg, 10.97 mmol) and the mixture was stirred at 0° C. for 2 h, then 6-(bromomethyl)-1-(4-methylbenzenesulfonyl)-1H-indole (4) (999 mg, 2.74 mmol) was added and the reaction mixture was stirred at 0° C. for another 2 h, allowed to warm to rt and stirred for 16 h. LCMS from the reaction mixture showed 25% of the desired product. The reaction mixture was purified by HPLC (SYSTEM 0-2-8 min 33-40-65% H₂O/ACN 30 ml\min (loading pump 4 ml ACN), target mass 403 column: XBRIDGE BEH C18 SMB100-5T 100*19 mm, 5 microM) to afford tert-butyl (2S,4R)-2-(dimethylcarbamothioyl)-4-[(1H-indol-6-yl)methoxy]pyrrolidine-1-carboxylate (6) (87 mg, 98.0% purity, 211.28 μmol, 7.7% yield).

### Step 5. Synthesis of (2S,4R)-4-[(1H-indol-6-yl)methoxy]-N,N-dimethylpyrrolidine-2-carbothioamide

Tert-butyl (2S,4R)-2-(dimethylcarbamothioyl)-4-[(1H-indol-6-yl)methoxy]pyrrolidine-1-carboxylate (6) (87 mg, 215.59 μmol) and dibromozinc (239 mg, 1.08 mmol) were mixed in ACN and stirred at 82° C. for 16 h. After 16 h the mixture was filtered, the mother liquor was evapourated to obtain (2S,4R)-4-[(1H-indol-6-yl)methoxy]-N,N-dimethylpyrrolidine-2-carbothioamide (7) (60 mg, 47.0% purity, 92.94 μmol, 43.1% yield).

### Step 6. Synthesis of (2S,4R)-2-(dimethylcarbamothioyl)-4-[(1H-indol-6-yl)methoxy]-N-[(2-methyl-4-4-methyl-2,4,5,9-tetraazatricyclo[8.4.0.0,3,7]tetradeca-1(14),3(7),5,10,12-pentaene-9-carbonylphenyl)methyl]pyrrolidine-1-carboxamide

1-(2-Methyl-4-4-methyl-2,4,5,9-tetraazatricyclo[8.4.0.0,3,7]tetradeca-1(14),3(7),5,10,12-pentaene-9-carbonylphenyl)methanamine (8) (49 mg, 141.25 μmol), 1-(1H-imidazole-1-carbonyl)-1H-imidazole (46 mg, 282.49 μmol) and ethylbis(propan-2-yl)amine (182 mg, 1.41 mmol) were dissolved in DMF (5 ml) and stirred at 15° C. for 3 h. (2S,4R)-4-[(1H-indol-6-yl)methoxy]-N,N-dimethylpyrrolidine-2-carbothioamide (7) (60 mg, 197.74 μmol) was added to the mixture and it was stirred at rt for 16 h. LCMS from the reaction mixture showed 10% of the desired product. The reaction mixture was purified by HPLC (SYSTEM 7-15-35% 0-2-8 min H₂O/ACN, flow 30 ml/min ((loading pump 4 ml ACN) target mass 676 column: XBridge BEH C18 100*19 mm, 5 microM) to afford 5.4 mg 68% purity, which were repurified by micro-HPLC (SYSTEM (modified after loading) Sample Info: 15 mM NH₄HCO3-ACN 30-50% ACN 0.5-6 min 5 ml/min. Chromatorex Phenyl 7.8*75 mm, 5 mkm) to afford (2S,4R)-2-(dimethylcarbamothioyl)-4-[(1H-indol-6-yl)methoxy]-N-[(2-methyl-4-4-methyl-2,4,5,9-tetraazatricyclo[8.4.0.0,3,7]tetradeca-1(14),3(7),5,10,12-pentaene-9-carbonylphenyl)methyl]pyrrolidine-1-carboxamide, LIT-003-12. Yield: 800 mg, 0.8%; Appearance: Yellow solid; HPLC purity: 97.11%; LCMS Calculated for C₃₇H₄₀N₈O₃S: 677.34; Observed: 677.4[M+H]⁺.

### The synthesis of 5-chloro-N-(3-chloro-5-cyclopropylphenyl)-2-methoxy-4-methylbenzene-1-sulfonamide (LIT-003-13)

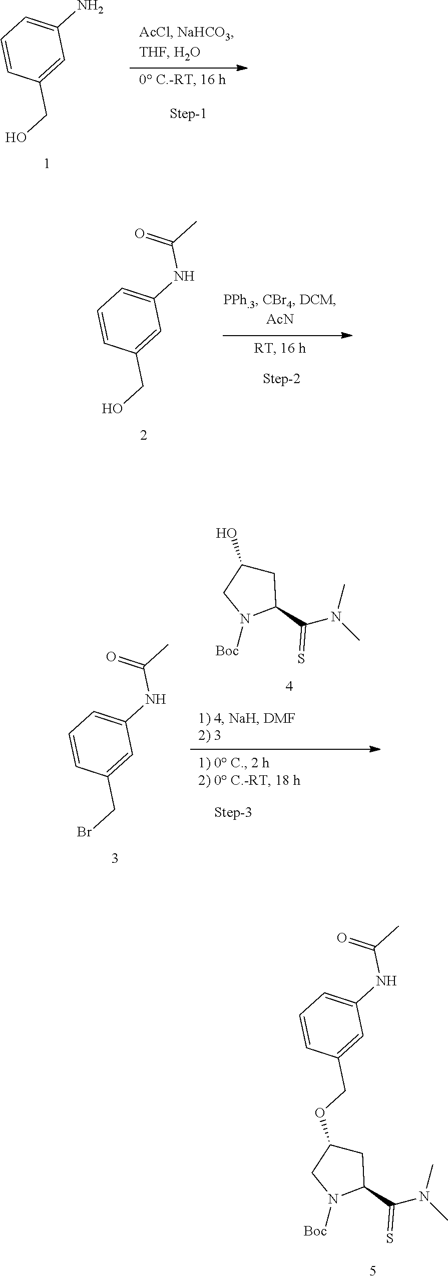 [SMILES: Nc1cccc(CO)c1 ; CC(=O)Nc1cccc(CO)c1 ; CC(=O)Nc1cccc(CBr)c1 ; CN(C)C(=S)[C@@H]1C[C@@H](O)CN1C(=O)OC(C)(C)C ; CC(=O)Nc1cccc(CO[C@@H]2C[C@@H](C(=S)N(C)C)N(C(=O)OC(C)(C)C)C2)c1]

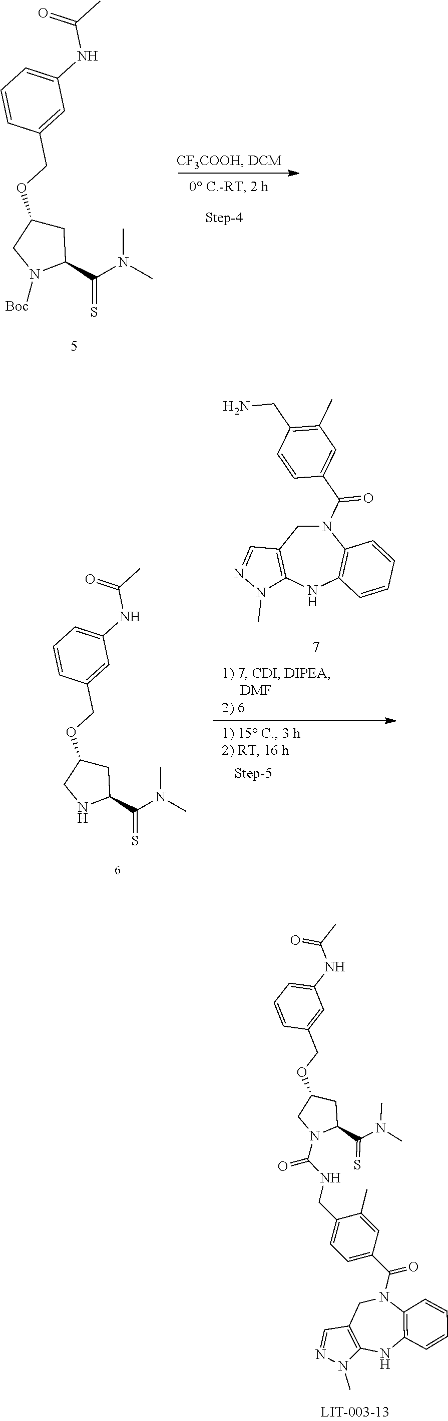 [SMILES: CC(=O)Nc1cccc(CO[C@@H]2C[C@@H](C(=S)N(C)C)N(C(=O)OC(C)(C)C)C2)c1 ; CC(=O)Nc1cccc(CO[C@H]2CN[C@H](C(=S)N(C)C)C2)c1 ; Cc1cc(C(=O)N2Cc3cnn(C)c3Nc3ccccc32)ccc1CN ; CC(=O)Nc1cccc(CO[C@@H]2C[C@@H](C(=S)N(C)C)N(C(=O)NCc3ccc(C(=O)N4Cc5cnn(C)c5Nc5ccccc54)cc3C)C2)c1]

### Step 1. Synthesis of N-[3-(hydroxymethyl)phenyl]acetamide

To a stirred solution of (3-aminophenyl)methanol (1) (5.0 g, 40.63 mmol) and sodium hydrogen carbonate (10.24 g, 121.88 mmol) in THF at 0° C. was added acetyl chloride (3.17 g, 40.63 mmol) dropwise and the resulting mixture was stirred at rt for 16 h. After 16 h the mixture was poured into water and the precipitate was filtered, washed with water and dried under reduced pressure to obtain N-[3-(hydroxymethyl)phenyl]acetamide (2) (4.8 g, 95.0% purity, 27.6 mmol, 67.9% yield).

### Step 2. Synthesis of N-[3-(bromomethyl)phenyl]acetamide

N-[3-(hydroxymethyl)phenyl]acetamide (2) (600 mg, 3.63 mmol) and triphenylphosphane (1.43 g, 5.45 mmol) were dissolved in DCM and the solution of tetrabromomethane (1.79 g, 5.45 mmol) in ACN was added dropwise at rt. The reaction mixture was stirred at rt for 16 h and concentrated. The residue was purified by FC (System ACN/CHCl3) to afford N-[3-(bromomethyl)phenyl]acetamide (3) (240 mg, 95.0% purity, 999.62 μmol, 27.5% yield).

### Step 3. Synthesis of tert-butyl (2S,4R)-2-(dimethylcarbamothioyl)-4-[(3-acetamidophenyl)methoxy]pyrrolidine-1-carboxylate

To a stirred solution of tert-butyl (2S,4R)-2-(dimethylcarbamothioyl)-4-hydroxypyrrolidine-1-carboxylate (4) (289 mg, 1.05 mmol) in DMF at 0° C. was added sodium hydride (101 mg, 4.21 mmol) and the mixture was stirred at 0° C. for 2 h, then N-[3-(bromomethyl)phenyl]acetamide (3) (239 mg, 1.05 mmol) was added and the reaction mixture was stirred at 0° C. for another 2 h, allowed to warm to rt and stirred for 16 h. LCMS from the reaction mixture showed 18% of the desired product. The reaction mixture was purified by HPLC (SYSTEM 0-2-8 min 23-30-100% H₂O/ACN, 30 ml\min (loading pump 4 ml ACN), target mass 421 column: Chromatorex 100*19 mm, 5 microM) to afford tert-butyl (2S,4R)-2-(dimethylcarbamothioyl)-4-[(3-acetamidophenyl)methoxy]pyrrolidine-1-carboxylate (5) (43 mg, 102.0 μmol, 9.7% yield).

### Step 4. Synthesis of N-[3-([(3R,5S)-5-(dimethylcarbamothioyl)pyrrolidin-3-yl]oxymethyl)phenyl]acetamide

Tert-butyl (2S,4R)-2-(dimethylcarbamothioyl)-4-[(3-acetamidophenyl)methoxy]pyrrolidine-1-carboxylate (5) (43 mg, 102.0 μmol) was dissolved in DCM (2 ml) and TFA (1 ml) was added to the mixture at 0° C. The resulting mixture was stirred at rt for 2 h. LCMS from the reaction mixture indicated 78% of the desired product. The mixture was concentrated to obtain N-[3-([(3R,5S)-5-(dimethylcarbamothioyl)pyrrolidin-3-yl]oxymethyl)phenyl]acetamide (6) (35 mg, 78.0% purity, 84.93 μmol, 83.2% yield) as a TFA salt.

### Step 5. Synthesis of (2S,4R)-2-(dimethylcarbamothioyl)-4-[(3-acetamidophenyl)methoxy]-N-[(2-methyl-4-4-methyl-2,4,5,9-tetraazatricyclo[8.4.0.0,3,7]tetradeca-1(14),3(7),5,10,12-pentaene-9-carbonylphenyl)methyl]pyrrolidine-1-carboxamide

1-(2-Methyl-4-4-methyl-2,4,5,9-tetraazatricyclo[8.4.0.0,3,7]tetradeca-1(14),3(7),5,10,12-pentaene-9-carbonylphenyl)methanamine (7) (27 mg, 77.72 μmol), 1-(1H-imidazole-1-carbonyl)-1H-imidazole (25 mg, 155.43 μmol) and ethylbis(propan-2-yl)amine (100 mg, 777.15 μmol) were dissolved in DMF (5 ml) and stirred at 15° C. for 3 h. N-[3-([(3R,5S)-5-(dimethylcarbamothioyl)pyrrolidin-3-yl]oxymethyl)phenyl]acetamide (6) (35 mg, 108.89 μmol) was added to the mixture and it was stirred at rt for 16 h. LCMS from the reaction mixture showed 91% of the desired product. The reaction mixture was purified by HPLC (SYSTEM 0-2-8 min 17-25-40% H2O/ACN 30 ml/min (loading pump 4 ml ACN), target mass 694 column: XBRIDGE BEH C18 SMB100-5T 100*19 mm, 5 microM) to afford (2S,4R)-2-(dimethylcarbamothioyl)-4-[(3-acetamidophenyl)methoxy]-N-[(2-methyl-4-4-methyl-2,4,5,9-tetraazatricyclo[8.4.0.0,3,7]tetradeca-1(14),3(7),5,10,12-pentaene-9-carbonylphenyl)methyl]pyrrolidine-1-carboxamide, LIT-003-13. Yield: 34.1 mg, 63.1%; Appearance: Beige solid; HPLC purity: 100%; LCMS Calculated for C₃H₄₂N₈O₄S: 695.35; Observed: 694.4[M+H]⁺.

### The synthesis of (2S,4R)-2-(dimethylcarbamothioyl)-N-[(2-methyl-4-4-methyl-2,4,5,9-tetraazatricyclo[8.4.0.0,3,7]tetradeca-1(14),3(7),5,10,12-pentaene-9-carbonylphenyl)methyl]-4-[3-(1H-1,2,3,4-tetrazol-5-yl)phenyl]methoxypyrrolidine-1-carboxamide (LIT-003-14)

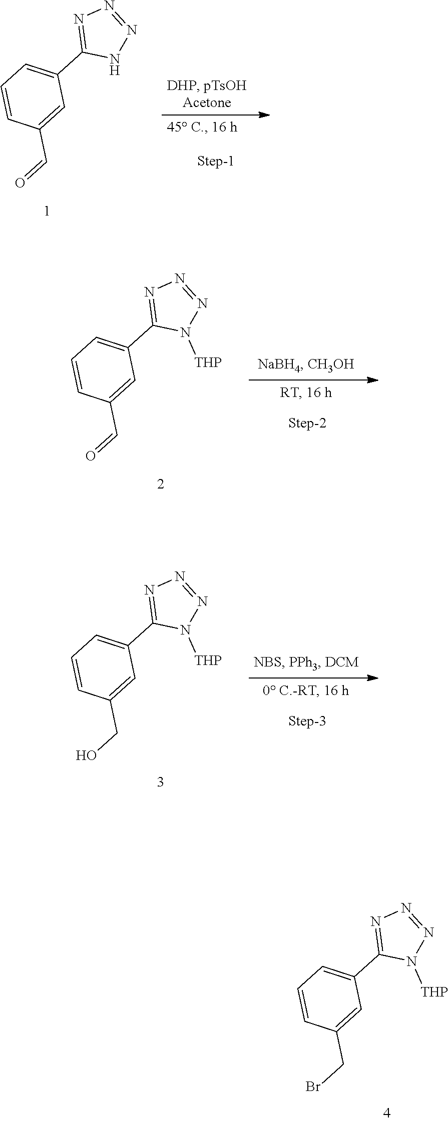

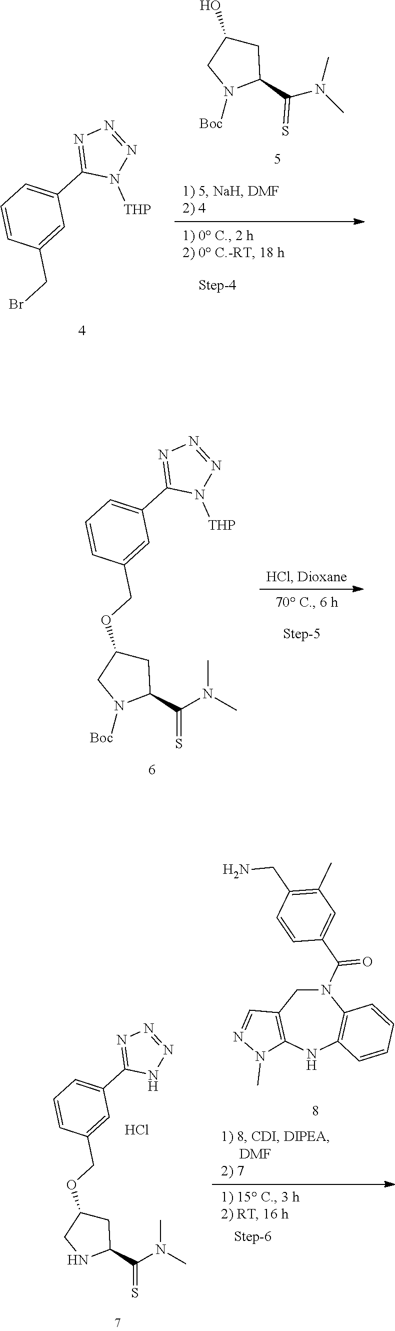

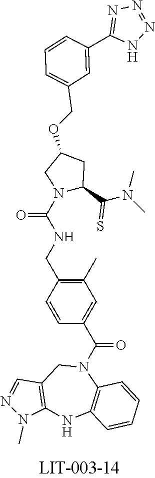 [SMILES: Cc1cc(C(=O)N2Cc3cnn(C)c3Nc3ccccc32)ccc1CNC(=O)N1C[C@H](OCc2cccc(-c3nnn[nH]3)c2)C[C@H]1C(=S)N(C)C]

### Step 1. Synthesis of 3-[1-(oxan-2-yl)-1H-1,2,3,4-tetrazol-5-yl]benzaldehyde

3-(1H-1,2,3,4-tetrazol-5-yl)benzaldehyde (1) (2.0 g, 11.49 mmol), 3,4-dihydro-2H-pyran (1.45 g, 17.24 mmol) and 4-methylbenzene-1-sulfonic acid hydrate (21.84 mg, 114.92 μmol) were mixed in acetone and stirred at 45° C. for 16 h. The reaction mixture was concentrated and the residue was purified by FC (system ACN/CHCl3) to afford 3-[1-(oxan-2-yl)-1H-1,2,3,4-tetrazol-5-yl]benzaldehyde (2) (2.8 g, 90.0% purity, 9.76 mmol, 84.9% yield).

### Step 2. Synthesis of 3-[1-(oxan-2-yl)-1H-1,2,3,4-tetrazol-5-yl]phenylmethanol

3-[1-(Oxan-2-yl)-1H-1,2,3,4-tetrazol-5-yl]benzaldehyde (2) (2.0 g, 7.75 mmol) was dissolved in MeOH and sodium borohydride (442 mg, 11.62 mmol) was added portionwise at rt. The mixture was stirred at rt for 16 h. LCMS from the reaction mixture indicated 84% of the desired product. The mixture was poured into water and extracted with EtOAc. The organic phase was washed with brine, dried over Na₂SO₄ and concentrated to obtain 3-[1-(oxan-2-yl)-1H-1,2,3,4-tetrazol-5-yl]phenylmethanol (3) (1.38 g, 84.0% purity, 4.45 mmol, 57.5% yield).

### Step 3. Synthesis of 5-[3-(bromomethyl)phenyl]-1-(oxan-2-yl)-1H-1,2,3,4-tetrazole

To a stirred solution of 3-[1-(oxan-2-yl)-1H-1,2,3,4-tetrazol-5-yl]phenylmethanol (3) (1.58 g, 6.07 mmol) in DCM at 0° C. was added triphenylphosphane (2.39 g, 9.11 mmol) followed by 1-bromopyrrolidine-2,5-dione (1.61 g, 9.11 mmol) and the resulting mixture was stirred at rt for 16 h. The mixture was concentrated and the residue was purified by FC (system ACN/CHCl3) to afford 5-[3-(bromomethyl)phenyl]-1-(oxan-2-yl)-1H-1,2,3,4-tetrazole (4) (900 mg, 90.0% purity, 2.51 mmol, 41.3% yield).

### Step 4. Synthesis of tert-butyl (2S,4R)-2-(dimethylcarbamothioyl)-4-(3-[1-(oxan-2-yl)-1H-1,2,3,4-tetrazol-5-yl]phenylmethoxy)pyrrolidine-1-carboxylate

To a stirred solution of tert-butyl (2S,4R)-2-(dimethylcarbamothioyl)-4-hydroxypyrrolidine-1-carboxylate (5) (467 mg, 1.7 mmol) in DMF at 0° C. was added sodium hydride (163 mg, 6.81 mmol) and the mixture was stirred at 0° C. for 2 h, then 5-[3-(bromomethyl)phenyl]-1-(oxan-2-yl)-1H-1,2,3,4-tetrazole (4) (550 mg, 1.7 mmol) was added and the reaction mixture was stirred at 0° C. for another 2 h, allowed to warm to rt and stirred for 16 h. LCMS from the reaction mixture showed 67% of the desired product. The reaction mixture was purified by HPLC (SYSTEM 0-2-8 min 38-45-60 H₂O/ACN 30 ml\min (loading pump 4 ml ACN), target mass 516 column: XBridge BEH C18 100*19 mm, 5 microM) to afford tert-butyl (2S,4R)-2-(dimethylcarbamothioyl)-4-(3-[1-(oxan-2-yl)-1H-1,2,3,4-tetrazol-5-yl]phenylmethoxy)pyrrolidine-1-carboxylate (6) (280 mg, 93.0% purity, 504.01 μmol, 29.6% yield).

### Step 5. Synthesis of (2S,4R)-N,N-dimethyl-4-[3-(1H-1,2,3,4-tetrazol-5-yl)phenyl]methoxypyrrolidine-2-carbothioamide hydrochloride

Tert-butyl (2S,4R)-2-(dimethylcarbamothioyl)-4-(3-[1-(oxan-2-yl)-1H-1,2,3,4-tetrazol-5-yl]phenylmethoxy)pyrrolidine-1-carboxylate (6) (63 mg, 121.94 μmol) was dissolved in 4M Dioxane-HCl (10 ml) and stirred at 70° C. for 6 h. LCMS from the reaction mixture indicated 58% of the desired product. The reaction mixture was concentrated to obtain (2S,4R)-N,N-dimethyl-4-[3-(1H-1,2,3,4-tetrazol-5-yl)phenyl]methoxypyrrolidine-2-carbothioamide hydrochloride (7) (40 mg, 58.0% purity, 62.89 μmol, 51.6% yield).

### Step 6. Synthesis of (2S,4R)-2-(dimethylcarbamothioyl)-N-[(2-methyl-4-4-methyl-2,4,5,9-tetraazatricyclo[8.4.0.0,3,7]tetradeca-1(14),3(7),5,10,12-pentaene-9-carbonylphenyl)methyl]-4-[3-(1H-1,2,3,4-tetrazol-5-yl)phenyl]methoxypyrrolidine-1-carboxamide

1-(2-Methyl-4-4-methyl-2,4,5,9-tetraazatricyclo[8.4.0.0,3,7]tetradeca-1(14),3(7),5,10,12-pentaene-9-carbonylphenyl)methanamine (8) (27 mg, 77.5 μmol), 1-(1H-imidazole-1-carbonyl)-1H-imidazole (25 mg, 154.99 μmol) and ethylbis(propan-2-yl)amine (100 mg, 774.97 μmol) were dissolved in DMF (5 ml) and stirred at 15° C. for 3 h. (2S,4R)-N,N-dimethyl-4-[3-(1H-1,2,3,4-tetrazol-5-yl)phenyl]methoxypyrrolidine-2-carbothioamide hydrochloride (7) (40 mg, 108.43 μmol) was added to the mixture and it was stirred at rt for 16 h. LCMS from the reaction mixture showed 68% of the desired product. The reaction mixture was purified by HPLC (SYSTEM 0-2-8 min 0-0-35% H2O/ACN/0.1% NH4OH 30 ml\min (loading pump 4 ml ACN), target mass 705 column: XBRIDGE BEH C18 100*19 mm, 5 microM) to afford (2S,4R)-2-(dimethylcarbamothioyl)-N-[(2-methyl-4-4-methyl-2,4,5,9-tetraazatricyclo[8.4.0.0,3,7]tetradeca-1(14),3(7),5,10,12-pentaene-9-carbonylphenyl)methyl]-4-[3-(1H-1,2,3,4-tetrazol-5-yl)phenyl]methoxypyrrolidine-1-carboxamide, LIT-003-14. Yield: 35.3 mg, 64.5%; Appearance: Beige solid; HPLC purity: 100%; LCMS Calculated for C₃₆H₃₉N₁₁O₃S: 706.33; Observed: 706.2[M+H]⁺.

### The synthesis of (2S,4R)-2-(dimethylcarbamothioyl)-N-[(2-methyl-4-4-methyl-2,4,5,9-tetraazatricyclo[8.4.0.0,3,7]tetradeca-1(14),3(7),5,10,12-pentaene-9-carbonylphenyl)methyl]-4-[(1H-1,2,3,4-tetrazol-5-yl)methoxy]pyrrolidine-1-carboxamide (LIT-003-15)

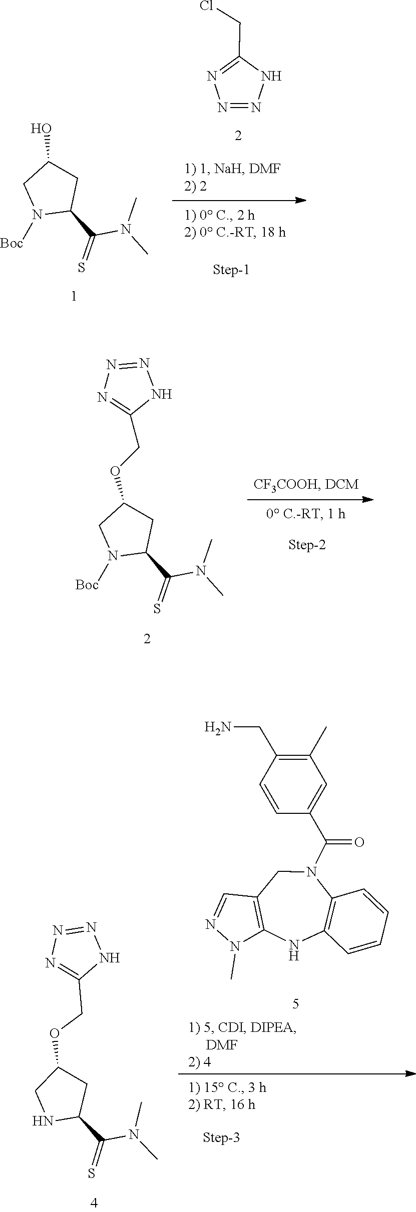 [SMILES: ClCc1nnn[nH]1 ; CN(C)C(=S)[C@@H]1C[C@@H](O)CN1C(=O)OC(C)(C)C ; CN(C)C(=S)[C@@H]1C[C@@H](OCc2nnn[nH]2)CN1C(=O)OC(C)(C)C ; CN(C)C(=S)[C@@H]1C[C@@H](OCc2nnn[nH]2)CN1 ; Cc1cc(C(=O)N2Cc3cnn(C)c3Nc3ccccc32)ccc1CN]

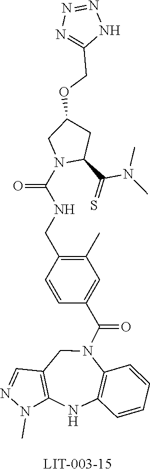 [SMILES: Cc1cc(C(=O)N2Cc3cnn(C)c3Nc3ccccc32)ccc1CNC(=O)N1C[C@H](OCc2nnn[nH]2)C[C@H]1C(=S)N(C)C]

### Step 1. Synthesis of tert-butyl (2S,4R)-2-(dimethylcarbamothioyl)-4-[(2H-1,2,3,4-tetrazol-5-yl)methoxy]pyrrolidine-1-carboxylate

To a stirred solution of tert-butyl (2S,4R)-2-(dimethylcarbamothioyl)-4-hydroxypyrrolidine-1-carboxylate (1) (500 mg, 1.82 mmol) in DMF at 0° C. was added sodium hydride (175 mg, 7.29 mmol) and the mixture was stirred at 0° C. for 2 h, then (1H-1,2,3,4-tetrazol-5-yl)methanol (2) (182 mg, 1.82 mmol) was added and the reaction mixture was stirred at 0° C. for another 2 h, allowed to warm to rt and stirred for 16 h. LCMS from the reaction mixture showed 35% of the desired product. The reaction mixture was purified by HPLC (SYSTEM 0-2-8 min 3-10-40% H₂O/ACN/0.1% FA 30 ml/min (loading pump 4 ml ACN), target mass 356 column: CHROMATOREX C18 SMB100-5T 100*19 mm, 5 microM) to afford tert-butyl (2S,4R)-2-(dimethylcarbamothioyl)-4-[(2H-1,2,3,4-tetrazol-5-yl)methoxy]pyrrolidine-1-carboxylate (3) (115 mg, 98.0% purity, 316.18 μmol, 17.3% yield).

### Step 2. Synthesis of (2S,4R)-N,N-dimethyl-4-[(2H-1,2,3,4-tetrazol-5-yl)methoxy]pyrrolidine-2-carbothioamide

Tert-butyl (2S,4R)-2-(dimethylcarbamothioyl)-4-[(2H-1,2,3,4-tetrazol-5-yl)methoxy]pyrrolidine-1-carboxylate (3) (55 mg, 154.3 μmol) was dissolved in DCM (2 ml) and TFA (1 ml) was added to the mixture at 0° C. The resulting mixture was stirred at rt for 1 h. LCMS from the reaction mixture indicated 37% of the desired product. The mixture was concentrated to obtain (2S,4R)-N,N-dimethyl-4-[(2H-1,2,3,4-tetrazol-5-yl)methoxy]pyrrolidine-2-carbothioamide (4) (40 mg, 37.0% purity, 57.74 μmol, 37.4% yield) as a TFA salt.

### Step 3. Synthesis of (2S,4R)-2-(dimethylcarbamothioyl)-N-[(2-methyl-4-4-methyl-2,4,5,9-tetraazatricyclo[8.4.0.0,3,7]tetradeca-1(14),3(7),5,10,12-pentaene-9-carbonylphenyl)methyl]-4-[(1H-1,2,3,4-tetrazol-5-yl)methoxy]pyrrolidine-1-carboxamide

1-(2-Methyl-4-4-methyl-2,4,5,9-tetraazatricyclo[8.4.0.0,3,7]tetradeca-1(14),3(7),5,10,12-pentaene-9-carbonylphenyl)methanamine (5) (39 mg, 111.48 μmol), 1-(1H-imidazole-1-carbonyl)-1H-imidazole (36 mg, 222.95 μmol) and ethylbis(propan-2-yl)amine (144 mg, 1.11 mmol) were dissolved in DMF (5 ml) and stirred at 15° C. for 3 h. (2S,4R)-N,N-dimethyl-4-[(2H-1,2,3,4-tetrazol-5-yl)methoxy]pyrrolidine-2-carbothioamide (4) (40 mg, 156.05 μmol) was added to the mixture and it was stirred at rt for 16 h. LCMS from the reaction mixture showed 42% of the desired product. The reaction mixture was purified by HPLC (SYSTEM 0-2-8 min 0-5-45% H₂O/MEOH/0.1% NH₄OH 30 ml\min (loading pump 4 ml MEOH), target mass 629 column: XBRIDGE BEH C18 SMB100-5T 100*19 mm, 5 microM) to afford (2S,4R)-2-(dimethylcarbamothioyl)-N-[(2-methyl-4-4-methyl-2,4,5,9-tetraazatricyclo[8.4.0.0,3,7]tetradeca-1(14),3(7),5,10,12-pentaene-9-carbonylphenyl)methyl]-4-[(1H-1,2,3,4-tetrazol-5-yl)methoxy]pyrrolidine-1-carboxamide, LIT-003-15. Yield: 25.0 mg, 33.8%; Appearance: White solid; HPLC purity: 97.34%; LCMS Calculated for C₃₀H₃₅N₁₁O₃S: 630.29; Observed: 630.4[M+H]⁺.

### The synthesis of 5-chloro-N-(3-chloro-5-cyclopropylphenyl)-2-methoxy-4-methylbenzene-1-sulfonamide (LIT-003-16)

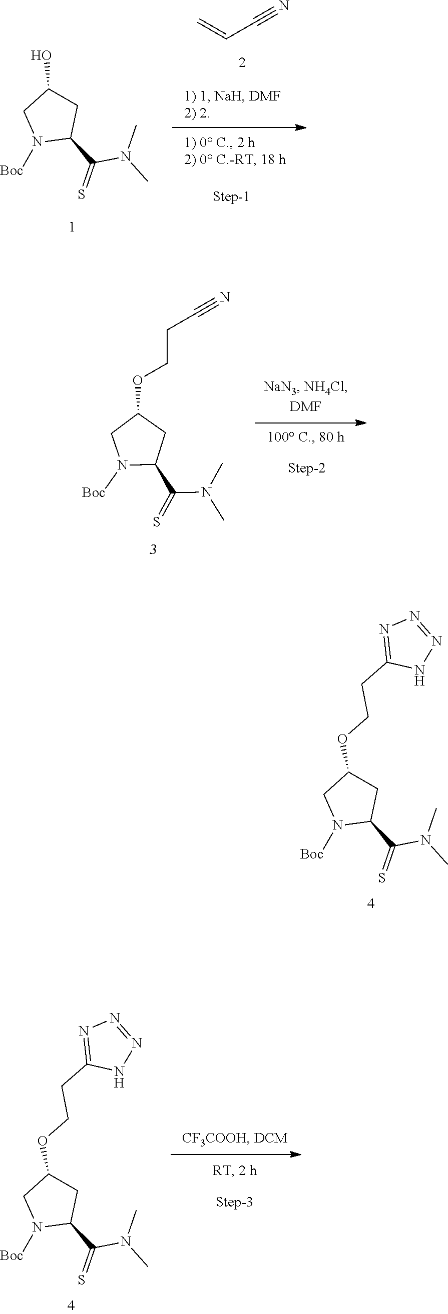

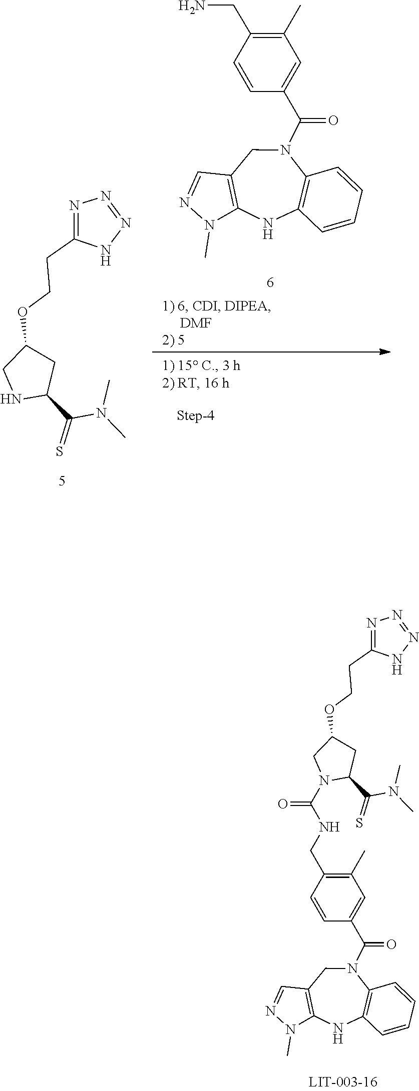 [SMILES: CN(C)C(=S)[C@@H]1C[C@@H](OCCc2nnn[nH]2)CN1 ; Cc1cc(C(=O)N2Cc3cnn(C)c3Nc3ccccc32)ccc1CN ; Cc1cc(C(=O)N2Cc3cnn(C)c3Nc3ccccc32)ccc1CNC(=O)N1C[C@H](OCCc2nnn[nH]2)C[C@H]1C(=S)N(C)C]

### Step 1. Synthesis of tert-butyl (2S,4R)-4-(2-cyanoethoxy)-2-(dimethylcarbamothioyl)pyrrolidine-1-carboxylate

To a stirred solution of tert-butyl (2S,4R)-2-(dimethylcarbamothioyl)-4-hydroxypyrrolidine-1-carboxylate (1) (500 mg, 1.82 mmol) in DMF at 0° C. was added sodium hydride (175 mg, 7.29 mmol) and the mixture was stirred at 0° C. for 2 h, then prop-2-enenitrile (2) (961 mg, 1.82 mmol) was added and the reaction mixture was stirred at 0° C. for another 2 h, allowed to warm to rt and stirred for 16 h. LCMS from the reaction mixture showed 57% of the desired product. The reaction mixture was purified by HPLC (SYSTEM 0-2-8 min 7-15-35% H₂O/ACN 30 ml/min (loading pump 4 ml ACN), target mass 327 column: Chromatorex C18 SMB100-5T 100*19 mm, 5 microM) to afford tert-butyl (2S,4R)-4-(2-cyanoethoxy)-2-(dimethylcarbamothioyl)pyrrolidine-1-carboxylate (3) (160 mg, 488.64 μmol, 26.8% yield).

### Step 2. Synthesis of tert-butyl (2S,4R)-2-(dimethylcarbamothioyl)-4-[2-(1H-1,2,3,4-tetrazol-5-yl)ethoxy]pyrrolidine-1-carboxylate

Tert-butyl (2S,4R)-4-(2-cyanoethoxy)-2-(dimethylcarbamothioyl)pyrrolidine-1-carboxylate (3) (160 mg, 488.64 μmol), sodium azide (95 mg, 1.47 mmol) and ammonium chloride (52 mg, 977.14 μmol) were mixed in DMF and stirred at 100° C. for 80 h. LCMS from the reaction mixture indicated 79% of the desired product. The mixture was concentrated to obtain tert-butyl (2S,4R)-2-(dimethylcarbamothioyl)-4-[2-(1H-1,2,3,4-tetrazol-5-yl)ethoxy]pyrrolidine-1-carboxylate (4) (60 mg, 79.0% purity, 127.95 μmol, 26.2% yield).

### Step 3. Synthesis of (2S,4R)-N,N-dimethyl-4-[2-(1H-1,2,3,4-tetrazol-5-yl)ethoxy]pyrrolidine-2-carbothioamide

Tert-butyl (2S,4R)-2-(dimethylcarbamothioyl)-4-[2-(1H-1,2,3,4-tetrazol-5-yl)ethoxy]pyrrolidine-1-carboxylate (4) (60 mg, 161.96 μmol) was dissolved in DCM (2 ml) and TFA (1 ml) was added to the mixture at 0° C. The resulting mixture was stirred at rt for 2 h. LCMS from the reaction mixture indicated 63% of the desired product. The mixture was concentrated to obtain (2S,4R)-N, N-dimethyl-4-[2-(1H-1,2,3,4-tetrazol-5-yl)ethoxy]pyrrolidine-2-carbothioamide (5) (40 mg, 63.0% purity, 93.21 μmol, 57.5% yield) as a TFA salt.

### Step 4. Synthesis of (2S,4R)-2-(dimethylcarbamothioyl)-N-[(2-methyl-4-4-methyl-2,4,5,9-tetraazatricyclo[8.4.0.0,3,7]tetradeca-1(14),3(7),5,10,12-pentaene-9-carbonylphenyl)methyl]-4-[2-(1H-1,2,3,4-tetrazol-5-yl)ethoxy]pyrrolidine-1-carboxamide

1-(2-Methyl-4-4-methyl-2,4,5,9-tetraazatricyclo[8.4.0.0,3,7]tetradeca-1(14),3(7),5,10,12-pentaene-9-carbonylphenyl)methanamine (6) (37 mg, 105.63 μmol), 1-(1H-imidazole-1-carbonyl)-1H-imidazole (34 mg, 211.26 μmol) and ethylbis(propan-2-yl)amine (136 mg, 1.06 mmol) were dissolved in DMF (5 ml) and stirred at 15° C. for 3 h. (2S,4R)-N, N-dimethyl-4-[2-(1H-1,2,3,4-tetrazol-5-yl)ethoxy]pyrrolidine-2-carbothioamide (5) (40 mg, 147.95 μmol) was added to the mixture and it was stirred at RT for 16 h. LCMS from the reaction mixture showed 43% of the desired product. The reaction mixture was purified by HPLC (SYSTEM 0-2-8 min 23-30-55% H2O/ACN 30 ml\min (loading pump 4 ml ACN), target mass 643 column: Chromatorex C18 SMB100-5T 100*19 mm, 5 microM) to afford (2S,4R)-2-(dimethylcarbamothioyl)-N-[(2-methyl-4-4-methyl-2,4,5,9-tetraazatricyclo[8.4.0.0,3,7]tetradeca-1(14),3(7),5,10,12-pentaene-9-carbonylphenyl)methyl]-4-[2-(1H-1,2,3,4-tetrazol-5-yl)ethoxy]pyrrolidine-1-carboxamide, LIT-003-16. Yield: 18.0 mg, 25.7%; Appearance: White solid; HPLC purity: 100%; LCMS Calculated for C₃₁H₃₇N₁₁O₃S: 644.31; Observed: 644.4[M+H]⁺.

### The synthesis of (2S,4R)-2-(dimethylcarbamothioyl)-4-[2-(2,5-dioxoimidazolidin-4-yl)ethoxy]-N-[(2-methyl-4-4-methyl-2,4,5,9-tetraazatricyclo[8.4.0.0,3,7]tetradeca-1(14),3(7),5,10,12-pentaene-9-carbonylphenyl)methyl]pyrrolidine-1-carboxamide (LIT-003-17)

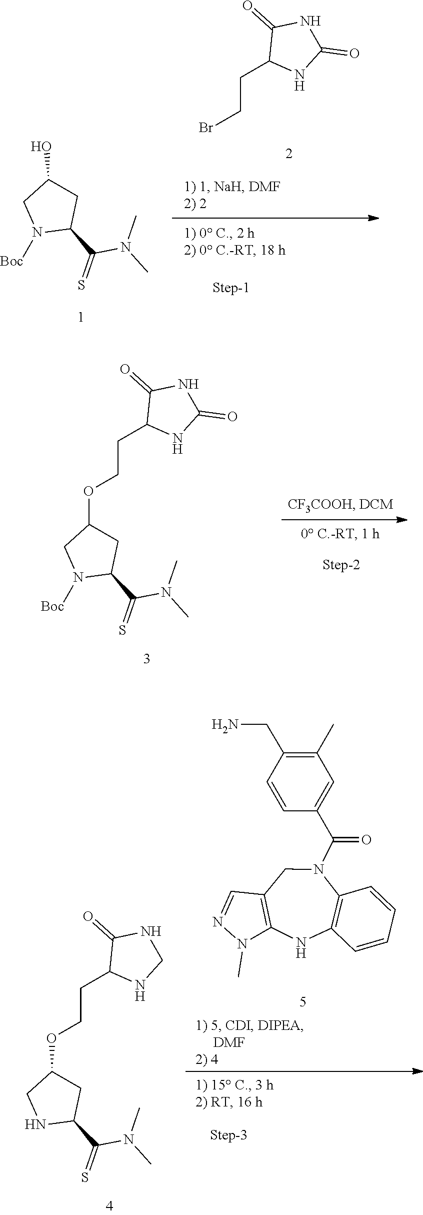 [SMILES: O=C1NC(=O)C(CCBr)N1 ; CN(C)C(=S)[C@@H]1C[C@@H](O)CN1C(=O)OC(C)(C)C ; CN(C)C(=S)[C@@H]1CC(OCCC2NC(=O)NC2=O)CN1C(=O)OC(C)(C)C ; CN(C)C(=S)[C@@H]1C[C@@H](OCCC2NCNC2=O)CN1 ; Cc1cc(C(=O)N2Cc3cnn(C)c3Nc3ccccc32)ccc1CN]

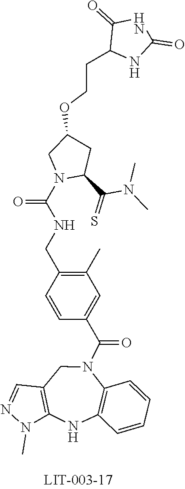 [SMILES: Cc1cc(C(=O)N2Cc3cnn(C)c3Nc3ccccc32)ccc1CNC(=O)N1C[C@H](OCCC2NC(=O)NC2=O)C[C@H]1C(=S)N(C)C]

### Step 1. Synthesis of tert-butyl (2S,4R)-2-(dimethylcarbamothioyl)-4-[2-(2,5-dioxoimidazolidin-4-yl)ethoxy]pyrrolidine-1-carboxylate

To a stirred solution of tert-butyl (2S,4R)-2-(dimethylcarbamothioyl)-4-hydroxypyrrolidine-1-carboxylate (1) (500 mg, 1.82 mmol) in DMF at 0° C. was added sodium hydride (175 mg, 7.29 mmol) and the mixture was stirred at 0° C. for 2 h, then 5-(2-bromoethyl)imidazolidine-2,4-dione (2) (375 mg, 1.82 mmol) was added and the reaction mixture was stirred at 0° C. for another 2 h, allowed to warm to rt and stirred for 16 h. LCMS from the reaction mixture showed 18% of the desired product. The reaction mixture was purified by HPLC (SYSTEM 0-2-8 min 0-0-100% H₂O/ACN 30 ml\min (loading pump 4 ml ACN), target mass 400 column: CHROMATOREX C18 100*19 mm, 5 microM) to afford tert-butyl (2S,4R)-2-(dimethylcarbamothioyl)-4-[2-(2,5-dioxoimidazolidin-4-yl)ethoxy]pyrrolidine-1-carboxylate (3) (37 mg, 90.0% purity, 83.15 μmol, 4.6% yield).

### Step 2. Synthesis of 2S,4R)-4-[2-(2,5-dioxoimidazolidin-4-yl)ethoxy]-N,N-dimethylpyrrolidine-2-carbothioamide

Tert-butyl (2S,4R)-2-(dimethylcarbamothioyl)-4-[2-(2,5-dioxoimidazolidin-4-yl)ethoxy]pyrrolidine-1-carboxylate (3) (37 mg, 92.39 μmol) was dissolved in DCM (2 ml) and TFA (1 ml) was added to the mixture at 0° C. The resulting mixture was stirred at rt for 1 h. LCMS from the reaction mixture indicated 88% of the desired product. The mixture was concentrated to obtain (2S,4R)-4-[2-(2,5-dioxoimidazolidin-4-yl)ethoxy]-N,N-dimethylpyrrolidine-2-carbothioamide (4) (30 mg, 88.0% purity, 87.89 μmol, 95% yield) as a TFA salt.

### Step 3. Synthesis of (2S,4R)-2-(dimethylcarbamothioyl)-4-[2-(2,5-dioxoimidazolidin-4-yl)ethoxy]-N-[(2-methyl-4-4-methyl-2,4,5,9-tetraazatricyclo[8.4.0.0,3,7]tetradeca-1(14),3(7),5,10,12-pentaene-9-carbonylphenyl)methyl]pyrrolidine-1-carboxamide

1-(2-Methyl-4-4-methyl-2,4,5,9-tetraazatricyclo[8.4.0.0,3,7]tetradeca-1(14),3(7),5,10,12-pentaene-9-carbonylphenyl)methanamine (5) (25 mg, 71.39 μmol), 1-(1H-imidazole-1-carbonyl)-1H-imidazole (23 mg, 142.78 μmol) and ethylbis(propan-2-yl)amine (92 mg, 713.88 μmol, 120.0 μl, 10.0 equiv) were dissolved in DMF (5 ml) and stirred at 15° C. for 3 h. (2S,4R)-4-[2-(2,5-dioxoimidazolidin-4-yl)ethoxy]-N,N-dimethylpyrrolidine-2-carbothioamide (4) (30 mg, 99.87 μmol) was added to the mixture and it was stirred at rt for 16 h. LCMS from the reaction mixture showed 56% of the desired product. The reaction mixture was purified by HPLC (SYSTEM 0-2-8 min 8-15-40% H₂O/ACN 30 ml\min (loading pump 4 ml ACN), target mass 673 column: Chromatorex C18 100*19 mm, 5 microM) to afford (2S,4R)-2-(dimethylcarbamothioyl)-4-[2-(2,5-dioxoimidazolidin-4-yl)ethoxy]-N-[(2-methyl-4-4-methyl-2,4,5,9-tetraazatricyclo[8.4.0.0,3,7]tetradeca-1(14),3(7),5,10,12-pentaene-9-carbonylphenyl)methyl]pyrrolidine-1-carboxamide, LIT-003-17. Yield: 11.5 mg, 23.4%; Appearance: Beige solid; HPLC purity: 98.11%; LCMS Calculated for C₃₃H₃₉N₉O₅S: 674.31; Observed: 674.4[M+H]⁺.

### The synthesis of 3-([(3R,5S)-5-(dimethylcarbamothioyl)-1-[(2-methyl-4-4-methyl-2,4,5,9-tetraazatricyclo[8.4.0.0,3,7]tetradeca-1(14),3(7),5,10,12-pentaene-9-carbonylphenyl)methyl]carbamoylpyrrolidin-3-yl]oxymethyl)benzene-1-sulfonic acid

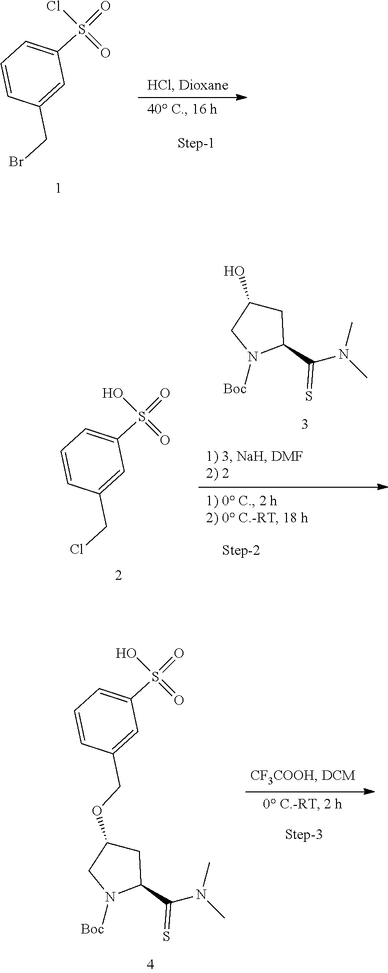 [SMILES: O=S(=O)(Cl)c1cccc(CBr)c1 ; O=S(=O)(O)c1cccc(CCl)c1 ; CN(C)C(=S)[C@@H]1C[C@@H](O)CN1C(=O)OC(C)(C)C ; CN(C)C(=S)[C@@H]1C[C@@H](OCc2cccc(S(=O)(=O)O)c2)CN1C(=O)OC(C)(C)C]

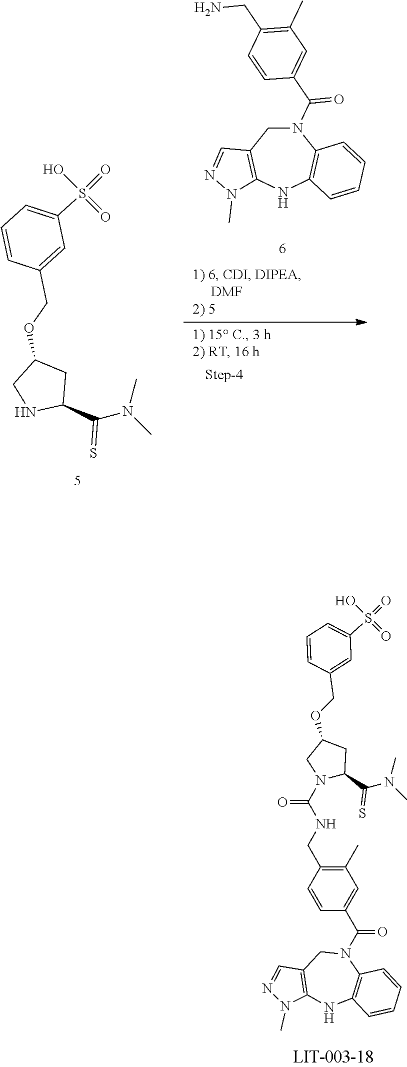 [SMILES: CN(C)C(=S)[C@@H]1C[C@@H](OCc2cccc(S(=O)(=O)O)c2)CN1 ; Cc1cc(C(=O)N2Cc3cnn(C)c3Nc3ccccc32)ccc1CN ; Cc1cc(C(=O)N2Cc3cnn(C)c3Nc3ccccc32)ccc1CNC(=O)N1C[C@H](OCc2cccc(S(=O)(=O)O)c2)C[C@H]1C(=S)N(C)C]

### Step 1. Synthesis of 3-(chloromethyl)benzene-1-sulfonic acid

3-(Bromomethyl)benzene-1-sulfonyl chloride (1) (200 mg, 746.56 μmol) was dissolved in Dioxane (10 ml) and HCl (10 ml) and was stirred at 40° C. for 16 h. The reaction mixture was concentrated. Water and CHCl₃ were added to the residue, water layer was separated and evapourated to obtain 3-(chloromethyl)benzene-1-sulfonic acid (2) (130 mg, 87.0% purity, 547.31 μmol, 60.5% yield).

### Step 2. Synthesis of 3-([(3R,5S)-1-[(tert-butoxy)carbonyl]-5-(dimethylcarbamothioyl)pyrrolidin-3-yl]oxymethyl)benzene-1-sulfonic acid

To a stirred solution of tert-butyl (2S,4R)-2-(dimethylcarbamothioyl)-4-hydroxypyrrolidine-1-carboxylate (3) (130 mg, 473.8 μmol) in DMF at 0° C. was added sodium hydride (46 mg, 1.9 mmol) and the mixture was stirred at 0° C. for 2 h, then 3-(chloromethyl)benzene-1-sulfonic acid (2) (98 mg, 474.62 μmol) was added and the reaction mixture was stirred at 0° C. for another 2 h, allowed to warm to rt and stirred for 16 h. LCMS from the reaction mixture showed 47% of the desired product. The reaction mixture was purified by HPLC (SYSTEM 0-2-8 min 0-0-25% H₂O/ACN 30 ml\min (loading pump 4 ml ACN), target mass 444 column: CHROMATOREX C18 100*19 mm, 5 microM) to afford 3-([(3R,5S)-1-[(tert-butoxy)carbonyl]-5-(dimethylcarbamothioyl)pyrrolidin-3-yl]oxymethyl)benzene-1-sulfonic acid (4) (67 mg, 98.0% purity, 147.69 μmol, 31.1% yield).

### Step 3. Synthesis of 3-([(3R,5S)-5-(dimethylcarbamothioyl)pyrrolidin-3-yl]oxymethyl)benzene-1-sulfonic acid

3-([(3R,5S)-1-[(tert-butoxy)carbonyl]-5-(dimethylcarbamothioyl)pyrrolidin-3-yl]oxymethyl)benzene-1-sulfonic acid (4) (67 mg, 150.71 μmol) was dissolved in DCM (2 ml) and TFA (1 ml) was added to the mixture at 0° C. The resulting mixture was stirred at rt for 2 h. LCMS from the reaction mixture indicated 90% of the desired product. The mixture was concentrated to obtain 3-([(3R,5S)-5-(dimethylcarbamothioyl)pyrrolidin-3-yl]oxymethyl)benzene-1-sulfonic acid (5) (50 mg, 90.0% purity, 130.64 μmol, 86.7% yield) as a TFA salt.

### Step 4. Synthesis of 3-([(3R,5S)-5-(dimethylcarbamothioyl)-1-[(2-methyl-4-4-methyl-2,4,5,9-tetraazatricyclo[8.4.0.0,3,7]tetradeca-1(14),3(7),5,10,12-pentaene-9-carbonylphenyl)methyl]carbamoylpyrrolidin-3-yl]oxymethyl)benzene-1-sulfonic acid

1-(2-Methyl-4-4-methyl-2,4,5,9-tetraazatricyclo[8.4.0.0,3,7]tetradeca-1(14),3(7),5,10,12-pentaene-9-carbonylphenyl)methanamine (6) (36 mg, 103.64 μmol), 1-(1H-imidazole-1-carbonyl)-1H-imidazole (34 mg, 207.28 μmol) and ethylbis(propan-2-yl)amine (134 mg, 1.04 mmol) were dissolved in DMF (5 ml) and stirred at 15° C. for 3 h. 3-([(3R,5S)-5-(dimethylcarbamothioyl)pyrrolidin-3-yl]oxymethyl)benzene-1-sulfonic acid (5) (50 mg, 145.16 μmol) was added to the mixture and it was stirred at rt for 16 h. LCMS from the reaction mixture showed 90% of the desired product. The reaction mixture was purified by HPLC (SYSTEM 0-5-35% 0-2-8 min H₂O/ACN/0.034% HCl, flow 30 ml/min ((loading pump 4 ml ACN) target mass 717 column: Chromatorex C18 SMB100-5T 100*19 mm, 5 microM) to afford 3-([(3R,5S)-5-(dimethylcarbamothioyl)-1-[(2-methyl-4-4-methyl-2,4,5,9-tetraazatricyclo[8.4.0.0,3,7]tetradeca-1(14),3(7),5,10,12-pentaene-9-carbonylphenyl)methyl]carbamoylpyrrolidin-3-yl]oxymethyl)benzene-1-sulfonic acid as HCl salt, LIT-003-18. Yield: 18.0 mg, 23.7%; Appearance: White solid; HPLC purity: 98.71%; LCMS Calculated for C₃₅H₃₉N₇O₆S₂: 718.27; Observed: 718.2[M+H]⁺.

### The synthesis of methyl 3-([(3R,5S)-5-(dimethylcarbamothioyl)-1-[(2-methyl-4-4-methyl-2,4,5,9-tetraazatricyclo[8.4.0.0,3,7]tetradeca-1(14),3(7),5,10,12-pentaene-9-carbonylphenyl)methyl]carbamoylpyrrolidin-3-yl]oxymethyl)benzoate (LIT-003-19)

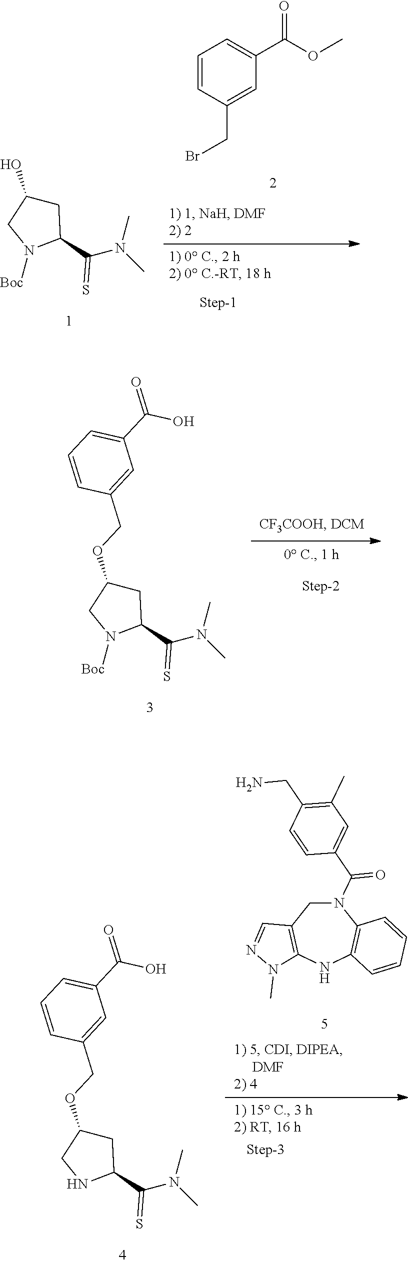 [SMILES: COC(=O)c1cccc(CBr)c1 ; CN(C)C(=S)[C@@H]1C[C@@H](O)CN1C(=O)OC(C)(C)C ; CN(C)C(=S)[C@@H]1C[C@@H](OCc2cccc(C(=O)O)c2)CN1C(=O)OC(C)(C)C ; CN(C)C(=S)[C@@H]1C[C@@H](OCc2cccc(C(=O)O)c2)CN1 ; Cc1cc(C(=O)N2Cc3cnn(C)c3Nc3ccccc32)ccc1CN]

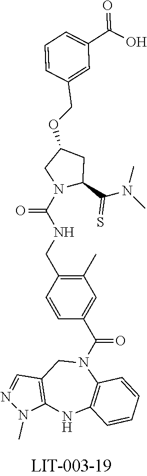 [SMILES: Cc1cc(C(=O)N2Cc3cnn(C)c3Nc3ccccc32)ccc1CNC(=O)N1C[C@H](OCc2cccc(C(=O)O)c2)C[C@H]1C(=S)N(C)C]

### Step 1. Synthesis of tert-butyl (2S,4R)-2-(dimethylcarbamothioyl)-4-[3-(methoxycarbonyl)phenyl]methoxypyrrolidine-1-carboxylate

To a stirred solution of tert-butyl (2S,4R)-2-(dimethylcarbamothioyl)-4-hydroxypyrrolidine-1-carboxylate (1) (250 mg, 911.15 μmol) in DMF at 0° C. was added sodium hydride (87 mg, 3.64 mmol) and the mixture was stirred at 0° C. for 2 h, then methyl 3-(bromomethyl)benzoate (2) (208 mg, 910.62 μmol) was added and the reaction mixture was stirred at 0° C. for another 2 h, allowed to warm to rt and stirred for 16 h. Water was added to the reaction mixture and left to stirred at rt for 2 h. LCMS from the reaction mixture showed 62% of the desired product. The reaction mixture was purified by HPLC (SYSTEM 0-0.2-3 min 0-0-20% H₂O/ACN 30 ml/min (loading pump 4 ml ACN), target mass 408 column: Chromatorex C18 SMB100-5T 100*19 mm, 5 microM) to afford 3-([(3R,5S)-1-[[(tert-butoxy)carbonyl]-5-(dimethylcarbamothioyl)pyrrolidin-3-yl]oxymethyl)benzoic acid (3) (160 mg, 391.67 μmol, 43% yield).

### Step 2. Synthesis of methyl 3-([(3R,5S)-5-(dimethylcarbamothioyl)pyrrolidin-3-yl]oxymethyl)benzoate

3-([(3R,5S)-1-[(tert-butoxy)carbonyl]-5-(dimethylcarbamothioyl)pyrrolidin-3-yl]oxymethyl)benzoic acid (3) (160 mg, 391.99 μmol) was dissolved in DCM (2 ml) and TFA (1 ml) was added to the mixture at 0° C. The resulting mixture was stirred at rt for 2 h. LCMS from the reaction mixture indicated 60% of the desired product. The mixture was concentrated to obtain 3-([(3R,5S)-5-(dimethylcarbamothioyl)pyrrolidin-3-yl]oxymethyl)benzoic acid (4) (120 mg, 60.0% purity, 233.47 μmol, 59.5% yield) as a TFA salt.

### Step 3. Synthesis of methyl 3-([(3R,5S)-5-(dimethylcarbamothioyl)-1-[(2-methyl-4-4-methyl-2,4,5,9-tetraazatricyclo[8.4.0.0,3,7]tetradeca-1(14),3(7),5,10,12-pentaene-9-carbonylphenyl)methyl]carbamoylpyrrolidin-3-yl]oxymethyl)benzoate

1-(2-Methyl-4-4-methyl-2,4,5,9-tetraazatricyclo[8.4.0.0,3,7]tetradeca-1(14),3(7),5,10,12-pentaene-9-carbonylphenyl)methanamine (5) (96 mg, 277.21 μmol), 1-(1H-imidazole-1-carbonyl)-1H-imidazole (90 mg, 554.41 μmol) and ethylbis(propan-2-yl)amine (358 mg, 2.77 mmol, 480.0 μl, 10.0 equiv) were dissolved in DMF (5 ml) and stirred at 15° C. for 3 h. 3-([(3R,5S)-5-(dimethylcarbamothioyl)pyrrolidin-3-yl]oxymethyl)benzoic acid (4) (120 mg, 389.11 μmol) was added to the mixture and it was stirred at rt for 16 h. LCMS from the reaction mixture showed 57% of the desired product. The reaction mixture was purified by HPLC (SYSTEM 0-2-8 min 43-50-75% H₂O/ACN/0.037% HCL 30 ml/min (loading pump 4 ml ACN), target mass 681 column: Chromatorex C18 SMB100-5T 100*19 mm, 5 microM) to afford 3-([(3R,5S)-5-(dimethylcarbamothioyl)-1-[(2-methyl-4-4-methyl-2,4,5,9-tetraazatricyclo[8.4.0.0,3,7]tetradeca-1(14),3(7),5,10,12-pentaene-9-carbonylphenyl)methyl]carbamoylpyrrolidin-3-yl]oxymethyl)benzoic acid as HCl salt, LIT-003-19. Yield: 6.4 mg, 3.4%; Appearance: Yellow solid; HPLC purity: 100%; LCMS Calculated for C₃₆H₄₀ClN₇O₅S: 682.31; Observed: 682.2[M+H]⁺.

### Example 2: Biological Assays

The activation constants (EC₅₀ in nM) were measured in CHO cell lines expressing oxytocin receptor by IP-One IP₁ accumulation assay. These values are the mean±SEM of at least three separate experiments performed in triplicate.

| 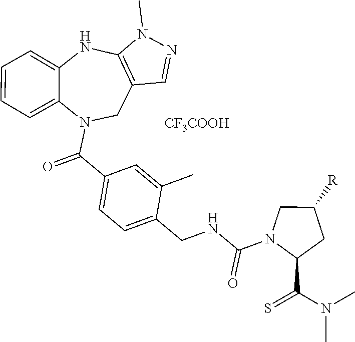 | | OT-R n = 3 |
|---|---|---|
| | | EC₅₀ |
| Compound | R | (nM) |
| Reference (not part of the invention) | —H | 45.1 |
| 1 | —F | 10.2 |
| 3 | 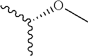 | 33 |
| 4 | 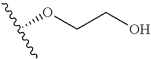 | 31.48 |
| 5 | 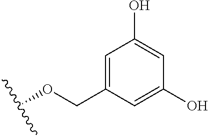 | 0.39 |
| 6 or LIT-003-01 | 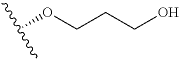 | 29.8 |
| 7 or LIT-003-02 | 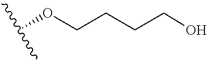 | 11.62 |
| 13 or LIT-003-09 | 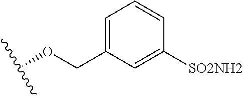 | 32.97 |
| 19 or LIT-003-15 | 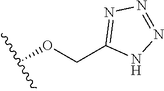 | 17.37 |
| 21 or LIT-003-17 | 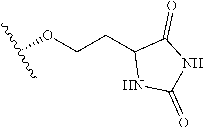 | 14.43 |

### Example 3: VIA/OT Selectivity Assays

Comparison of efficacy as OT receptor agonist (measured as described in example 2), or as VIA receptor antagonist (measured as calcium signal, as follows), has been performed.

| 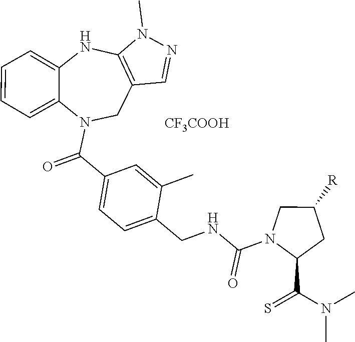 | | OT-R (Ago) n = 3 | V_{1A}-R (Antago) n = 3 | Ratio V_{1A}/OT n = 3 |
|---|---|---|---|---|
| | | EC₅₀ | IC₅₀ | |
| Ref | R | (nM) | (nM) | |
| Reference | —H | 45.1 | 346 | 7.7 |
| 1 | —F | 10.2 | 250 | 24 |
| 4 | 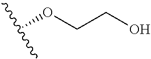 | 31.48 | 585 | 18.6 |
| 5 | 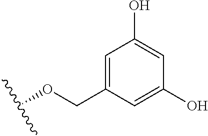 | 0.39 | 2432 | 6235 |

1. A compound of following formula (I): 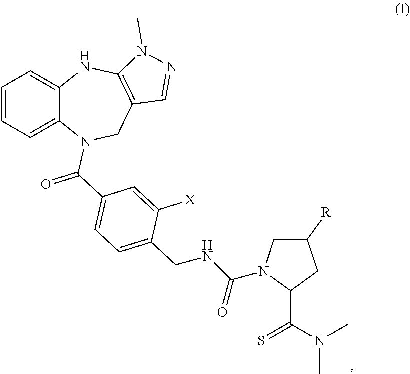
wherein:
X is chosen from —H, halogens, —(C₁-C₆)-alkyl groups, —O—(C₁-C₆)-alkyl groups, —S—(C₁-C₆)-alkyl groups and —O—CF₃, —S—CF₃, in particular -Me or —Cl, more particularly -Me;R is chosen from the group comprising:
—(C₁-C₆)-alkyl groups, in particular -Me;
—(C₁-C₆)-alkyl-V groups, in particular —CH₂—CH₂-V, with V being a halogen, in particular —F;
—O—(C₁-C₄)-alkyl groups, in particular —OMe;
—S—(C₁-C₄)-alkyl groups, in particular —SMe;
—O—(C₂-C₄)-alkyl-OH and —S—(C₂-C₄)-alkyl-OH groups, in particular —O—(CH₂)ᵢ—OH, with i ranging from 2 to 4, i being more particularly 2, 3 or 4;
—O(—(C₂-C₄)-alkyl-O)ₙ—H and —S(—(C₂-C₄)-alkyl-O)ₙ—H groups, in particular —O(—CH₂—CH₂—O)ₙ—H, with n ranging from 2 to 4, n being more particularly 2;
—O—(C₂-C₄)-alkyl-O—(C₁-C₄)-alkyl, —S—(C₂-C₄)-alkyl-O—(C₁-C₄)-alkyl, —O—(C₂-C₄)-alkyl-S—(C₁-C₄)-alkyl and —S—(C₂-C₄)-alkyl-S—(C₁-C₄)-alkyl groups, in particular —O—(CH₂)ᵢ—OMe, with i ranging from 2 to 4, i being more particularly 2;
—O—(C₂-C₄)-alkyl-O—(C₁-C₄)-alkyl-W″, —S—(C₂-C₄)-alkyl-O—(C₁-C₄)-alkyl-W″, —O—(C₂-C₄)-alkyl-S—(C₁-C₄)-alkyl-W″ and —S—(C₂-C₄)-alkyl-S—(C₁-C₄)-alkyl-W″ groups, with W″ being an halogen, in particular —F, for example —O—CH₂—CH₂—O—(CH₂)₂—F;
—O(—(C₂-C₄)-alkyl-O)ₘ—(C₁-C₄)-alkyl and —S(—(C₂-C₄)-alkyl-O)ₘ—(C₁-C₄)-alkyl groups, in particular —O(—CH₂—CH₂—O)ₘ-Me or —O(—CH₂—CH₂—O)ₘ-Et, with m ranging from 2 to 4, m being more particularly 2;
—O(—(C₂-C₄)-alkyl-O)ₘ—(C₁-C₄)-alkyl-W″—S(—(C₂-C₄)-alkyl-O)ₘ—(C₁-C₄)-alkyl-W″ groups, with W″ being an halogen, in particular —F, for example —O(—CH₂—CH₂—O)₂—(CH₂)₂—F;
—O—(C₁-C₆)-alkyl-V and —S—(C₁-C₆)-alkyl-V groups, in particular —CH₂—CH₂-V;
V being chosen from the group comprising:
—C(═O)NR_{d}R_{d}′, R_{d} and R_{d}′ being independently H or a —(C₁-C₆)-alkyl group, in particular H;
—S(═O)₂NR_{d}R_{d}′, R_{d} and R_{d}′ being independently H or a —(C₁-C₆)-alkyl group, in particular H; —C(═O)OR_{d}, with R_{d} being H or a —(C₁-C₆)-alkyl group;
—S(═O)₂OH;
halogens, in particular —F;
-cyclo-(C₃-C₆)-alkyl groups being optionally substituted by at least one group chosen from —(C₁-C₆)-alkyl and ═O groups; and
-heterocyclo-(C₃-C₆)-alkyl groups being optionally substituted by at least one group chosen from —(C₁-C₆)-alkyl and ═O groups, for example imidazolidine-2,4-dionyl;
said O—(C₁-C₆)-alkyl-V group being for example of the following formula: 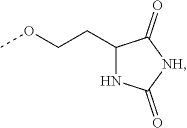
—O—(C₁-C₄)-alkyl-Ar, in particular —O—(CH₂)ⱼ-Ar, with j ranging from 1 to 4, j being more particularly 1 or 2, and Ar being an aryl, for example phenyl, or heteroaryl, for example indolyl, tetrazolyl, pyridinyl, pyrimidinyl, pyrazinyl or pyridazinyl, group being optionally substituted by at least one R″ group, in particular at least one —OH group,
R″ being chosen from the group comprising:
—OH;
halogens, in particular —F;
a —O—(C₁-C₆)-alkyl and —S—(C₁-C₆)-alkyl, in particular a —OMe, or —O-cyclo-(C₃-C₆)-alkyl, in particular a —O-cyclopropyl;
—O—(C₂-C₄)-alkyl-OH and —S—(C₂-C₄)-alkyl-OH groups, in particular —O—(CH₂)ᵢ—OH, with i ranging from 2 to 4, i being more particularly 2, 3 or 4;
—O(—(C₂-C₄)-alkyl-O)ₙ—H and —S(—(C₂-C₄)-alkyl-O)ₙ—H groups, in particular —O(—CH₂—CH₂—O)ₙ—H, with n ranging from 2 to 4, n being more particularly 2;
—O—(C₂-C₄)-alkyl-O—(C₁-C₄)-alkyl, —S—(C₂-C₄)-alkyl-O—(C₁-C₄)-alkyl, —O—(C₂-C₄)-alkyl-S—(C₁-C₄)-alkyl and —S—(C₂-C₄)-alkyl-S—(C₁-C₄)-alkyl groups, in particular —O—(CH₂)ᵢ—OMe, with i ranging from 2 to 4, i being more particularly 2;
—O—(C₂-C₄)-alkyl-O—(C₁-C₄)-alkyl-W″, —S—(C₂-C₄)-alkyl-O—(C₁-C₄)-alkyl-W″, —O—(C₂-C₄)-alkyl-S—(C₁-C₄)-alkyl-W″ and —S—(C₂-C₄)-alkyl-S—(C₁-C₄)-alkyl-W″ groups, with W″ being an halogen, in particular —F, for example —O—CH₂—CH₂—O—(CH₂)₂—F; —O(—(C₂-C₄)-alkyl-O)ₘ—(C₁-C₄)-alkyl groups, in particular —O(—CH₂—CH₂—O)ₘ-Me or —O(—CH₂—CH₂—O)ₘ-Et, with m ranging from 2 to 4, m being more particularly 2;
—O(—(C₂-C₄)-alkyl-O)ₘ—(C₁-C₄)-alkyl-W″ and —S(—(C₂-C₄)-alkyl-O)ₘ—(C₁-C₄)-alkyl-W″ groups, with W″ being an halogen, in particular —F, for example —O(—CH₂—CH₂—O)₂—(CH₂)₂—F;
a —(C₁-C₆)-alkyl or -cyclo-(C₃-C₆)-alkyl, in particular a cyclopropyl;
—(C₁-C₆)-alkyl-W groups, in particular —(CH₂)ₖ—W groups, said groups being optionally further substituted by at least a —W′ group, with k ranging from 1 to 6, k being more particularly 1 or 2, and W and W′ being an halogen, in particular —F, ORₐ, with Rₐ representing —H or —(C₁-C₄)-alkyl, in particular H or Me, or a PEG group, in particular of following formula —(CH₂—CH₂—O)ₗ—H, with l ranging from 1 to 10, in particular from 2 to 4, for example 2; or W and W′ being NR_{b}H, with R_{b} representing —H, a —(C₁-C₄)-alkyl group, or a —(C₁-C₄)-alkyl-Ar′, with Ar′ being an aryl or heteroaryl group, in particular a furanyl;
—(C₁-C₆)-alkyl-Y—Z groups, in particular —(CH₂)ₗ—Y—Z, with l ranging from 1 to 6, 1 being more particularly 1 or 2, Y being —O—, —NH— or being absent, and Z being chosen from saturated heterocyclic groups, in particular morpholinyl, tetrahydropyranyl, imidazolidinyl, said saturated heterocyclic groups being optionally substituted by at least one ═O group, representing for example imidazolidine-2,4-dione; and heteroaryl groups, in particular tetrazolyl;
an aryl or heteroaryl group, in particular a pyrazolyl or a tetrazolyl, said group being optionally substituted by at least one R_{c} group chosen from —(C₁-C₆)-alkyl and —NH₂ groups;
a —(C₀-C₆)-alkyl-C(═O)OR_{d}, with R_{d} being H or a —(C₁-C₆)-alkyl group, in particular H, —(C₀-C₆)-alkyl-C(═O)NR_{d}R_{d}′, R_{d} and R_{d}′ being independently H or a —(C₁-C₆)-alkyl group, in particular H, —(C₀-C₆)-alkyl-S(═O)₂OR_{d}, R_{d} being H or a —(C₁-C₆)-alkyl group, in particular H, or —(C₀-C₆)-alkyl-S(═O)₂NR_{d}R_{d}′ group, R_{d} and R_{d}′ being independently H, a —(C₁-C₆)-alkyl group, in particular H, or a —NH—C(═O)—C₁-C₆-alkyl group, in particular —NH—C(═O)-Me group, more particularly a —COOH, —C(═O)NH₂, —S(═O)₂₀H, —S(═O)₂NH₂ or —(CH₂)₂—S(═O)₂NH₂ group; a —NHC(═O)—(C₁-C₆)-alkyl, in particular —NHC(═O)-Me;
said —O—(C₁-C₄)-alkyl-Ar group being for example of one of the following formulae: 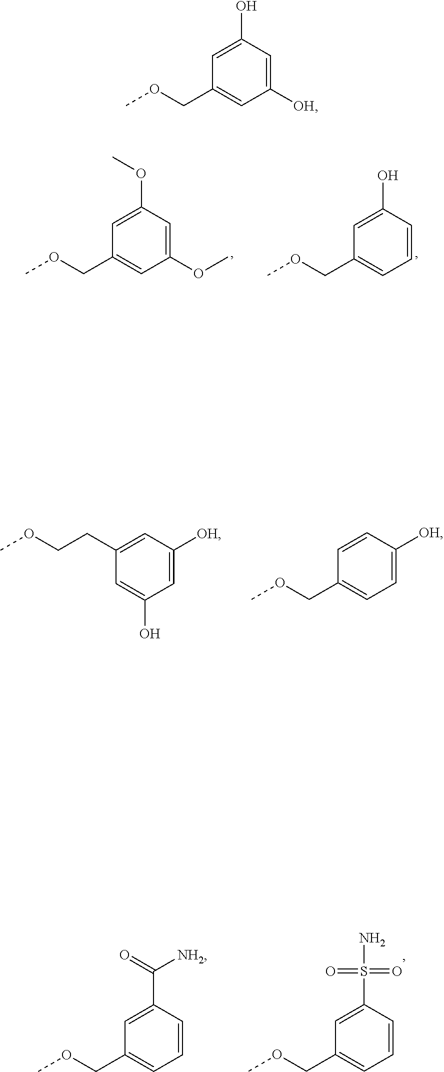 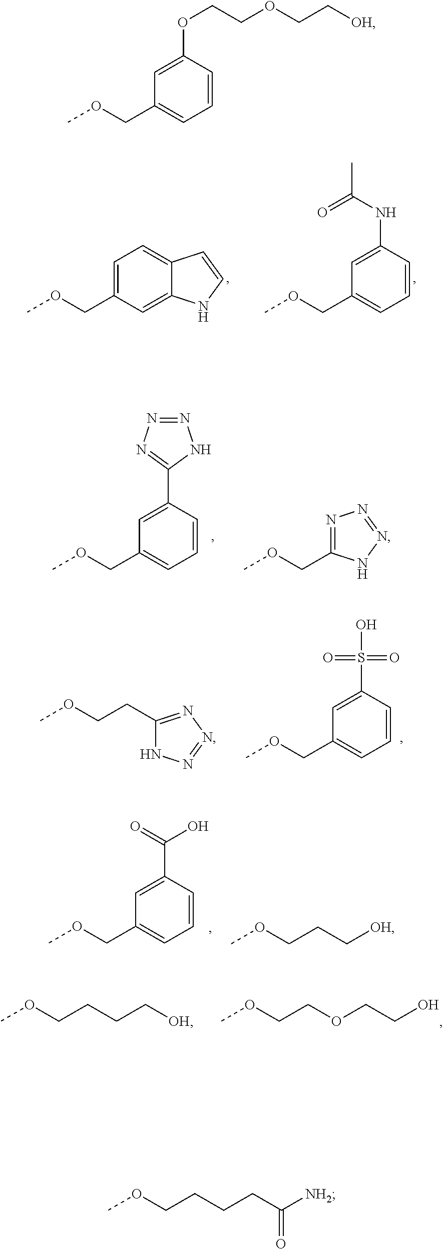
Halogens, in particular —F; and
—CF₃;
or R is —F and its geminal hydrogen is replaced by —F, R forming with the carbon atom C to which it is bound and its —F replaced geminal hydrogen a CF₂ residue;
or an enantiomer, diastereoisomer, and/or a pharmaceutically acceptable salt or solvate thereof.

2. The compound according to claim 1, of following formula (I): 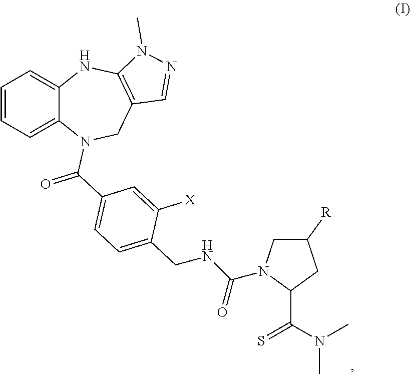
wherein:
X is -Me or —Cl, in particular -Me;
R is chosen from the group comprising:
—O—(C₁-C₄)-alkyl groups, in particular —OMe;
—O—(C₂-C₄)-alkyl-OH groups, in particular —O—(CH₂)ᵢ—OH, with i ranging from 2 to 4, i being more particularly 2;
—O(—(C₂-C₄)-alkyl-O)ₙ—H groups, in particular —O(—CH₂—CH₂)ₙ—OH, with n ranging from 2 to 4, n being more particularly 2;
—O—(C₂-C₄)-alkyl-O—(C₁-C₄)-alkyl groups, in particular —O—(CH₂)ᵢ—OMe, with i ranging from 2 to 4, i being more particularly 2;
—O(—(C₂-C₄)-alkyl-O)ₘ—(C₁-C₄)-alkyl groups, in particular —O(—CH₂—CH₂—O)ₘ-Me or —O(—CH₂—CH₂—O)ₘ-Et, with m ranging from 2 to 4, m being more particularly 2; and
—O—(C₁-C₄)-alkyl-Ar, in particular —O—(CH₂)ⱼ-Ar, with j ranging from 1 to 4, j being more particularly 1, and Ar being an aryl group being optionally substituted by at least one R″ group as defined in claim 1, in particular at least one —OH group, said —O—(C₁-C₄)-alkyl-Ar group being for example of the following formula: 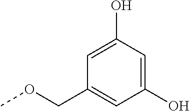
Halogens, in particular —F.

3. The compound according to claim 1, of following formula (I): 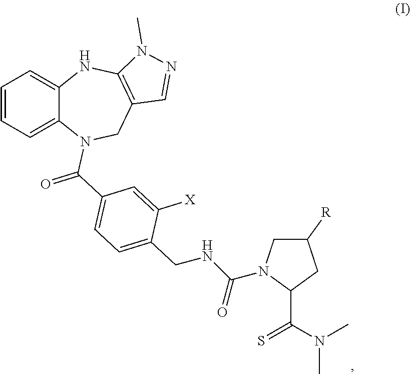
wherein:
X is -Me or —Cl, in particular -Me;
R is chosen from
—O—(C₁-C₄)-alkyl-Ar groups, in particular —O—(CH₂)ⱼ-Ar, with j ranging from 1 to 4, j being more particularly 1, and Ar being an aryl group being optionally substituted by at least one R″ group as defined in claim 1, in particular at least one —OH group, said —O—(C₁-C₄)-alkyl-Ar group being for example of the following formula:

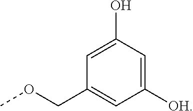

4. The compound according to claim 1, of one of the following formulae:

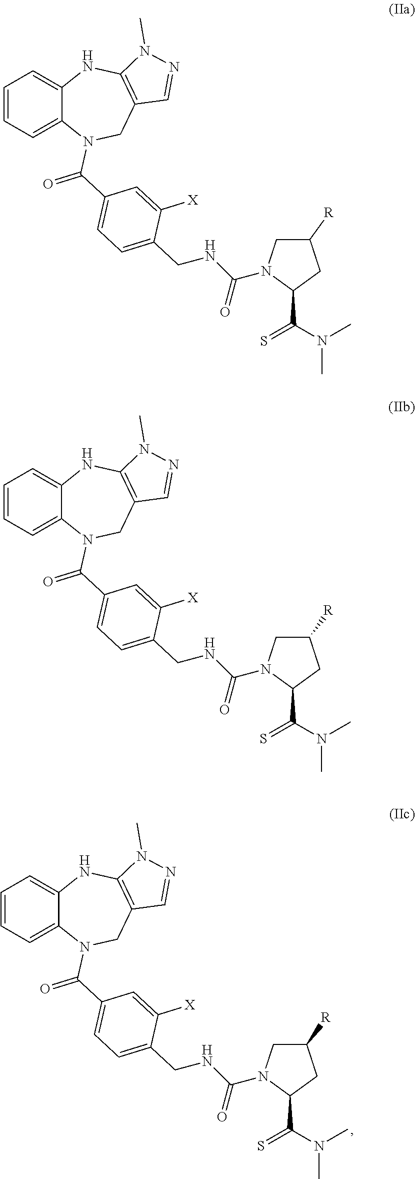

5. The compound according to claim 1, wherein X is Me.

6. The compound according to claim 1, wherein X is Cl.

7. The compound according to claim 1, wherein R is chosen from:

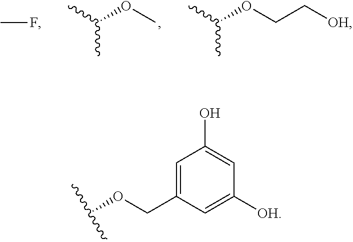

8. A pharmaceutical, dermatologic or cosmetic composition comprising a compound according to claim 1 in admixture with at least one pharmaceutical acceptable excipient.

9. The compound according to claim 1 for use in the prevention and/or treatment of a disease, condition or disorder, which is autism, autism spectrum disorders, in particular for the treatment of social interaction impairments in autism spectrum disorders, pain, drug and/or alcohol addiction, depression, post-traumatic stress, anxiety, eating disorders, notably anorexia, bulimia and Prader-Willi syndrome, schizophrenia, neurodevelopmental disorders, social interaction disorders, borderline behavior, ageing of the skin, muscles, bones and any disease for which oxytocin is known to have a beneficial effect.

10. A composition comprising at least one compound according to claim 1 and a diuretic, for example urea or its salt, or a V2r antagonist as a combination product for simultaneous, separate or spread therapeutic use in the prevention and/or treatment of a disease, condition or disorder, which is autism, autism spectrum disorders, in particular for the treatment of social interaction impairments in autism spectrum disorders, pain, drug and/or alcohol addiction, depression, post-traumatic stress, anxiety, eating disorders, notably anorexia, bulimia and Prader-Willi syndrome, schizophrenia, neurodevelopmental disorders, social interaction disorders, borderline behavior, ageing of the skin, muscles, bones and any disease for which oxytocin is known to have a beneficial effect.
